(19) United States
(12) Patent Application Publication
(10) Pub. No.: US 2025/0066837 A1
(43) Pub. Date: Feb. 27, 2025

(54) METHODS AND COMPOSITIONS FOR RAPID DETECTION AND ANALYSIS OF RNA AND DNA CYTOSINE METHYLATION

(71) Applicant: THE UNIVERSITY OF CHICAGO, Chicago, IL (US)

(72) Inventors: Chuan HE, Chicago, IL (US); Qing DAI, Chicago, IL (US); Iryna IRKLIYENKO, Chicago, IL (US); Chang YE, Chicago, IL (US)

(73) Assignee: THE UNIVERSITY OF CHICAGO, Chicago, IL (US)

(21) Appl. No.: 18/726,353

(22) PCT Filed: Jan. 6, 2023
(86) PCT No.: PCT/US2023/060267

§ 371 Date: Jul. 2, 2024

Related U.S. Application Data
(60) Provisional application No. 63/297,165, filed on Jan. 6, 2022.

(51) Int. Cl. C12Q 1/6806

(52) U.S. Cl. CPC C12Q 1/6806

(57) ABSTRACT

Aspects of the present disclosure are directed to methods, compositions, and kits for detection and analysis of DNA and RNA cytosine methylation. Certain aspects include methods, compositions and kits useful in bisulfite sequencing of methylated nucleic acids, including methylated nucleic acids from low-input samples such as cell-free DNA and cell-free RNA. Also disclosed are methods and compositions for detection and quantification of 5-hydroxymethylcytosine in DNA.

### CROSS-REFERENCE TO RELATED APPLICATIONS

This application claims the benefit of priority of U.S. Provisional Patent Application No. 63/297,165 filed Jan. 6, 2022, which is hereby incorporated by reference in its entirety.

### STATEMENT OF GOVERNMENT SUPPORT

This invention was made with government support under RM1 HG008935 awarded by the National Institutes of Health. The government has certain rights in the invention.

### SEQUENCE LISTING

The instant application contains a Sequence Listing which has been submitted in ST26 format and is hereby incorporated by reference in its entirety. Said ST26 copy, created on Jan. 6, 2023, is named ARCD_P0756WO_Sequence_Listing.xml and is 59,305 bytes in size.

### BACKGROUND

### I. Field of the Invention

Aspects of this invention relate to at least the fields of cell biology and epigenetics.

### II. Background

Bisulfite sequencing (BS-seq) is the current gold standard for 5mC sequencing in DNA and has also been widely used for m⁵C sequencing in RNA. However, conventional BS-seq suffers several major drawbacks, limiting its application in m⁵C sequencing in RNA and 5mC sequencing in DNA, especially for in low-input samples. There exists a need for improved methods and compositions for detection and analysis of DNA and RNA cytosine methylation, including DNA and RNA from low-input samples.

### SUMMARY

The present disclosure provides various methods, compositions, systems, and kits for nucleic acid processing and cytosine methylation analysis. Certain aspects of the disclosure are directed to particular bisulfite compositions useful in rapid bisulfite treatment of DNA and/or RNA for detection and analysis of 5mC and m⁵C. Also disclosed are DNA and RNA processing methods comprising use of the disclosed compositions for cytosine deamination and preparation of DNA and/or RNA for sequencing and cytosine methylation analysis. Further disclosed are methods for 5hmC detection, quantification, and analysis. DNA and RNA processing kits are disclosed, including bisulfite conversion kits useful in preparation of DNA and/or RNA for cytosine methylation analysis.

Aspects of the disclosure include bisulfite solutions, ammonium sulfite solutions, ammonium bisulfite solutions, bisulfite solutions that do not comprise sodium bisulfite, nucleic acid processing methods, DNA processing methods, RNA processing methods, methods for 5mC analysis, methods for m⁵C analysis, methods for 5hmC analysis, bisulfite sequencing methods, methylation analysis methods, bisulfite treatment methods, nucleic acid processing kits, DNA processing kits, and RNA processing kits. Methods of the disclosure can include at least 1, 2, 3, or more of the following steps: generating a bisulfite solution, mixing a first ammonium bisulfite solution and a second ammonium bisulfite solution, incubating a DNA molecule in a bisulfite solution, incubating an RNA molecule in a bisulfite solution, removing a DNA molecule from a bisulfite solution, removing an RNA molecule from a bisulfite solution, subjecting a DNA molecule to alkaline conditions, subjecting an RNA molecule to alkaline conditions, treating a DNA molecule with an APOBEC deaminase enzyme, detecting a nucleotide methylation, quantifying nucleotide methylation, obtaining a sample from a subject, isolating nucleic acid molecules from a sample, sequencing a DNA molecule, and sequencing an RNA molecule. Any one or more of the preceding steps may be excluded from certain aspects. Compositions (e.g., solutions) of the disclosure can include at least 1, 2, 3, or more of the following components: ammonium bisulfite, ammonium sulfite, sodium bisulfite, sodium hydroxide, and an APOBEC deaminase enzyme. Any one or more of the preceding components may be excluded from certain aspects. Kits of the disclosure can include at least 1, 2, 3, 4, or more of the following components: a bisulfite solution, a sodium bisulfite solution, an ammonium bisulfite solution, a bisulfite solution that does not comprise sodium bisulfite, an alkaline solution, a buffer, instructions for DNA processing, instructions for DNA processing, instructions for bisulfite treatment of DNA, and instructions for bisulfite treatment of RNA. Any one or more of the preceding components may be excluded from certain aspects.

Disclosed herein, in some aspects, is a method for DNA processing, the method comprising: (a) incubating a solution comprising a DNA molecule and ammonium bisulfite at a temperature of at least 95° C. for at most 12 minutes, wherein the solution does not comprise sodium bisulfite or added sodium bisulfite; and (b) subjecting the DNA molecule to alkaline conditions. Also disclosed, in some aspects, is a method for DNA processing, the method comprising: (a) generating a solution comprising a DNA molecule and ammonium bisulfite, wherein the solution does not comprise sodium bisulfite or added sodium bisulfite; (b) incubating the solution at a temperature of at least 95° C.; and (c) removing the DNA molecule from the solution at most 12 minutes after (a). Further disclosed, in some aspects, is a method for processing a nucleic acid sample, the method comprising incubating a solution comprising DNA molecules and ammonium bisulfite at a temperature of at least 95° C. for at most 12 minutes, wherein the solution does not comprise sodium bisulfite or added sodium bisulfite, wherein the DNA molecules each comprise one or more cytosine residues, wherein, after incubating the solution, greater than 99% of the DNA molecules comprise no cytosine residue. In some aspects, the method further comprises subjecting the plurality of DNA molecules to alkaline conditions. In some aspects, the solution does not comprise ammonium sulfite or added ammonium sulfite.

Disclosed herein, in some aspects, is a method for RNA processing, the method comprising (a) incubating a solution comprising an RNA molecule, ammonium sulfite, and ammonium bisulfite at a temperature of at least 95° C. for at most 12 minutes, wherein the solution does not comprise sodium bisulfite or added sodium bisulfite; (b) subjecting the RNA molecule to alkaline conditions. Also disclosed, in some aspects, is a method for RNA processing, the method comprising (a) generating a solution comprising an RNA molecule, ammonium sulfite, and ammonium bisulfite, wherein the solution does not comprise sodium bisulfite or added sodium bisulfite; (b) incubating the solution at a temperature of at least 95° C.; and (c) removing the RNA molecule from the solution at most 12 minutes after (a). Further disclosed, in some aspects, is a method for processing a nucleic acid sample, the method comprising incubating a solution comprising RNA molecules, ammonium sulfite, and ammonium bisulfite at a temperature of at least 95° C. for at most 12 minutes, wherein the solution does not comprise sodium bisulfite or added sodium bisulfite, wherein the RNA molecules each comprise one or more cytosine residues, wherein, after incubating the solution, greater than 99% of the RNA molecules comprise no cytosine residue. In some aspects, the method further comprises subjecting the plurality of RNA molecules to alkaline conditions. In some aspects, the solution comprises between 5% and 15% ammonium sulfite by weight. In some aspects, the solution comprises 5%, 6%, 7%, 8%, 9%, 10%, 11%, 12%, 13%, 14%, or 15% ammonium sulfite by weight, or any range or value derivable therein. In some aspects, the solution comprises about 10% ammonium sulfite by weight.

In some aspects, the solution comprises between 50% and 70% ammonium bisulfite by weight. In some aspects, the solution comprises, comprises at least, or comprises at most 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, or 70% ammonium bisulfite by weight, or any range or value derivable therein. In some aspects, the solution comprises between 65% and 67% ammonium bisulfite by weight. In some aspects, the solution comprises about 66.7% ammonium bisulfite by weight.

In some aspects, a solution does not comprise added sodium bisulfite. In some aspects, a solution does not comprise sodium bisulfite at levels greater than or equal to about 0.01%, 0.02%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 11%, 12%, 13%, 14%, 15%, 16%, 17%, 18%, 19%, or 20%, or any range derivable therein, relative to the levels of ammonium sulfite and/or ammonium bisulfite. In some aspects, a solution does not comprise sodium bisulfite at levels greater than or equal to about 0.01%, 0.02%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 11%, 12%, 13%, 14%, 15%, 16%, 17%, 18%, 19%, or 20% concentration, or any range derivable therein.

In some aspects, a solution does not comprise added ammonium sulfite. In some aspects, a solution does not comprise ammonium sulfite at levels greater than or equal to about 0.01%, 0.02%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 11%, 12%, 13%, 14%, 15%, 16%, 17%, 18%, 19%, or 20%, or any range derivable therein, relative to the levels of ammonium bisulfite. In some aspects, a solution does not comprise ammonium sulfite at levels greater than or equal to about 0.01%, 0.02%, 0.03%, 0.04%, 0.05%, 0.06%, 0.07%, 0.08%, 0.09%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1%, 2%, 3%, 4%, 5%, 6%, 7%, 8%, 9%, 10%, 11%, 12%, 13%, 14%, 15%, 16%, 17%, 18%, 19%, or 20% concentration, or any range derivable therein.

In some aspects, a solution does not comprise ammonium sulfite or added ammonium sulfite. In some aspects, the solution comprises ammonium sulfite at a concentration of, or of less than 1 M, 0.9 M, 0.8 M, 0.7 M, 0.6 M, 0.5 M, 0.4 M, 0.3 M, 0.2 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or less. In some aspects, the solution comprises less than 1%, 0.1%, 0.01%, 0.001%, or 0.0001% ammonium sulfite by weight, or less.

In some aspects, the solution does not comprise sodium bisulfite or added sodium bisulfite. In some aspects, the solution comprises sodium bisulfite at a concentration of, or of less than 1 M, 0.9 M, 0.8 M, 0.7 M, 0.6 M, 0.5 M, 0.4 M, 0.3 M, 0.2 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or less. In some aspects, the solution comprises less than 1%, 0.1%, 0.01%, 0.001%, or 0.0001% sodium bisulfite by weight, or less.

In some aspects, the solution is at a bisulfite concentration between 6.5 M and 10 M, or any range or value derivable therein. In some aspects, the solution is at a bisulfite concentration between 8 M and 10 M. In some aspects, the solution is at a bisulfite concentration between 9 M and 10 M. In some aspects, the solution is at a bisulfite concentration between 6.5 M and 7.5 M. In some aspects, the solution is at a bisulfite solution of, of at least, or of at most 6.5 M, 6.6 M, 6.7 M, 6.8 M, 6.9 M, 7 M, 7.1 M, 7.2 M, 7.3 M, 7.4 M, 7.5 M, 7.6 M, 7.7 M, 7.8 M, 7.9 M, 8 M, 8.1 M, 8.2 M, 8.3 M, 8.4 M, 8.5 M, 8.6 M, 8.7 M, 8.8 M, 8.9 M, 9 M, 9.1 M, 9.2 M, 9.3 M, 9.4 M, 9.5 M, 9.6 M, 9.7 M, 9.8 M, 9.9 M, 10 M, 10.1 M, 10.2 M, 10.3 M, 10.4 M, or 10.5 M, or any range or value derivable therein. In some aspects, the solution is at a bisulfite solution of about 7.0 M. In some aspects, the solution is at a bisulfite solution of about 9.5 M.

In some aspects, the solution has a pH between 4.8 and 5.4. In some aspects, the solution has a pH of, of at least, or of at most, 4.5, 4.6, 4.7, 4.8, 4.9, 5.0, 5.1, 5.2, 5.3, 5.4, 5.5, 5.6, 5.7, 5.8, 5.9, or more, or any range or value derivable therein. In some aspects, the solution has a pH of about 5.1.

In some aspects, the method comprises incubating the solution at a temperature of, of at least, or of at most 95° C., 96° C., 97° C., 98° C., 99° C., 99.5° C., 99.9° C., or any range or value derivable therein. In some aspects, the method comprises incubating the solution at a temperature of at least 98° C. In some aspects, the method comprises incubating the solution for, for at least, or for at most 12, 11, 10, 9, 8, 7, 6, 5, or 4 minutes, or any range or value derivable therein. In some aspects, the method comprises incubating the solution for at most 10 minutes. In some aspects, the method comprises incubating the solution for at most 8 minutes.

Also disclosed herein, in some aspects, is a DNA processing kit comprising (a) a solution comprising ammonium bisulfite having a bisulfite concentration between 6.5 M and 10 M, wherein the solution does not comprise sodium bisulfite or added sodium bisulfite; and (b) instructions for processing a DNA sample. In some aspects, the solution does not comprise ammonium sulfite or added ammonium sulfite. In some aspects, the kit further comprises an alkaline solution. In some aspects, the kit further comprises one or more buffer solutions. Any one or more of the preceding components may be excluded from certain aspects.

Further disclosed herein, in some aspects, is an RNA processing kit comprising (a) a solution comprising ammonium sulfite and ammonium bisulfite at a bisulfite concentration between 6.5 M-8 M, wherein the solution does not comprise sodium bisulfite or added sodium bisulfite; and (b) instructions for processing an RNA sample. In some aspects, the solution comprises between 5% and 15% ammonium sulfite by weight. In some aspects, the solution comprises, comprises at most, or comprises at least 5%, 6%, 7%, 8%, 9%, 10%, 11%, 12%, 13%, 14%, or 15% ammonium sulfite by weight, or any range or value derivable therein. In some aspects, the kit further comprises an alkaline solution. In some aspects, the kit further comprises one or more buffer solutions. Any one or more of the preceding components may be excluded from certain aspects.

In some aspects, the solution comprises between 50% and 70% ammonium bisulfite by weight. In some aspects, the solution comprises, comprises at least, or comprises at most 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, or 70% ammonium bisulfite by weight, or any range or value derivable therein. In some aspects, the solution comprises between 65% and 67% ammonium bisulfite by weight. In some aspects, the solution comprises about 66.7% ammonium bisulfite by weight.

In some aspects, the solution is at a bisulfite concentration between 6.5 M and 10 M, or any range or value derivable therein. In some aspects, the solution is at a bisulfite concentration between 8 M and 10 M. In some aspects, the solution is at a bisulfite concentration between 9 M and 10 M. In some aspects, the solution is at a bisulfite concentration between 6.5 M and 7.5 M. In some aspects, the solution is at a bisulfite concentration of 6.5 M, 6.6 M, 6.7 M, 6.8 M, 6.9 M, 7 M, 7.1 M, 7.2 M, 7.3 M, 7.4 M, 7.5 M, 7.6 M, 7.7 M, 7.8 M, 7.9 M, 8 M, 8.1 M, 8.2 M, 8.3 M, 8.4 M, 8.5 M, 8.6 M, 8.7 M, 8.8 M, 8.9 M, 9 M, 9.1 M, 9.2 M, 9.3 M, 9.4 M, 9.5 M, 9.6 M, 9.7 M, 9.8 M, 9.9 M, 10 M, 10.1 M, 10.2 M, 10.3 M, 10.4 M, or 10.5 M, or any range or value derivable therein. In some aspects, the solution is at a bisulfite concentration of about 7.0 M. In some aspects, the solution is at a bisulfite concentration of about 9.5 M.

In some aspects, the solution has a pH between 4.8 and 5.4. In some aspects, the solution has a pH of, of at least, or of at most, 4.5, 4.6, 4.7, 4.8, 4.9, 5.0, 5.1, 5.2, 5.3, 5.4, 5.5, 5.6, 5.7, 5.8, or 5.9, or any range or value derivable therein. In some aspects, the solution has a pH of about 5.1.

In some aspects, the instructions comprise instructions for incubating the DNA sample with the solution at a temperature of, or of at least 95° C., 96° C., 97° C., 98° C., 99° C., 99.5° C., 99.9° C., or any range or value derivable therein. In some aspects, the instructions comprise instructions for incubating the DNA sample with the solution at a temperature of at least 98° C. In some aspects, the instructions comprise instructions for incubating the DNA sample with the solution at most 12, 11, 10, 9, 8, 7, 6, 5, or 4 minutes, or any range or value derivable therein. In some aspects, the instructions comprise instructions for incubating the DNA sample with the solution at most 10 minutes. In some aspects, the instructions comprise instructions for incubating the DNA sample with the solution at most 8 minutes.

Also disclosed herein, in some aspects, is a method for 5-hydroxymethylcytosine analysis, the method comprising (a) incubating a first solution comprising a first DNA molecule and ammonium bisulfite at a temperature of at least 95° C. for at most 12 minutes; (b) incubating a second solution comprising a second DNA molecule and ammonium bisulfite at a temperature of at least 95° C. for at most 12 minutes; (c) subjecting the first DNA molecule to alkaline conditions; (d) subjecting the second DNA molecule to alkaline conditions; (e) treating the second DNA molecule with an APOBEC deaminase enzyme; (f) sequencing the first DNA molecule and the second DNA molecule. In some aspects, the first solution does not comprise sodium bisulfite. In some aspects, the second solution does not comprise sodium bisulfite; In some aspects, the first solution and the second solution are the same solution. In some aspects, the first solution and the second solution are different solutions. In some aspects, (a) and (b) are performed simultaneously. In some aspects, (c) and (d) are performed simultaneously. In some aspects, the first DNA molecule and the second DNA molecule have the same nucleotide sequence. In some aspects, the APOBEC deaminase enzyme is APOBEC3A.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

A person of ordinary skill in the art would understand that a solution that does not contain a particular chemical (e.g., ammonium sulfite, sodium bisulfite, etc.) does not contain an added quantity of that chemical. The term added means that the chemical is exogenously supplied, i.e. supplied in amounts greater than what would be considered trace or minute amounts.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1 shows a diagram or the mechanism of bisulfite sequencing reactions.

FIG. 2A shows matrix-assisted laser desorption/ionization time of flight mass spectrometry (Maldi-TOF MS) monitoring the reaction of AGCGA (SEQ ID NO: 1) with R-1G at 98° C., showing that cytosine was completely converted to U-BS adduct within 3 min. Upon base treatment, U-BS adduct was converted to U quantitatively. FIG. 2B shows Maldi-TOF MS monitoring the reaction of AGm⁵CGA (SEQ ID NO: 2) with R-1G at 98° C., showing that m⁵C did not react with R-1G even after 30 minutes of incubation.

FIG. 3 shows RNA fragment size distribution after treatment with R-1G for different length of times (min) at 95° C. or 98° C. In all cases, the RNA fragments are distributed between 150 to 300 bp.

FIG. 4 shows sequencing results for total RNA from A549 cells, with the impacts of reaction temperature and reaction time (X-axis) on mean mutation rates for known-m⁵C sites (top, Y-axis) and non-m⁵C sites (bottom, Y-axis) graphically presented. Conditions with suitably low levels of mean mutation of non-m⁵C sites and suitably high levels of mean mutation rate of known-m⁵C sites were identified. The condition of 9 minutes at 98° C. provided results characteristic of improvements described in this disclosure, these conditions can be noted as “Opti. conditions” and/or “D.5” in certain portions of this disclosure.

FIG. 5 shows m⁵C detection levels at 28S rRNA sites. The 28S rRNA m⁵C sites and background sites can act as benchmarks for m⁵C detection assay sensitivity and background measurements. There are two completely modified m⁵C sites on 28S rRNA (marked with vertical line), and all other C sites are unmodified. As shown with conditions of 9 minutes at 98° C., the non-conversion rates for the two known m⁵C sites were over 95%, while the non-conversion rates for all of the C sites were below 5%.

FIGS. 6A-6D show false positive sites in various BS sequencing methods. The X axis represents position in 28S human rRNA. The Y axis represents the detected C ratio. The red dots represent the false positive sites, the green dots represent the known msC sites (marked with vertical lines). FIG. 6A shows results from a canonical-BS treatment (e.g., “Zymo kit”).

FIG. 6B shows results from the methods of Yang et al.¹⁰. FIG. 6C shows results from the methods of Huang et al.¹⁵ FIG. 6D shows results from the methods of Zhang et al.²¹

FIGS. 7A-7F show analysis of various treatment times and temperatures using R-1G recipe and validation using 28S rRNA. FIG. 7A shows false positive rate on non-m⁵C sites under different conditions. A.1: 70° C. for 40 min, B.1: 80° C. for 30 min, B.2: 80° C. for 60 min, B.3: 80° C. for 120 min, C.1: 90° C. for 20 min, C.2: 90° C. for 30 min, C.3: 90° C. for 45 min, C.4: 90° C. for 60 min, D.1: 98° C. for 5 min, D.2: 98° C. for 6 min, D.3: 98° C. for 7 min, D.4: 98° C. for 8 min, D.5: 98° C. for 9 min, D.6: 98° C. for 10 min, D.7: 98° C. for 15 min. FIG. 7B shows detected methylation ratio on the two known m⁵C sites under different time and temperature. FIG. 7C shows that, under conditions D5, the two known m⁵C sites showed high detection rates while the all the false positive rates were under 5%. FIG. 7D shows sequence depth at different position on 28S rRNA. FIG. 7E and FIG. 7F show statistics of the false positive rates (FP) and detected m⁵C site fractions in the different noted methods ((.g., Zymo EZ RNA Methylation™ kit (“Zymo Kit”), Yang et al., 2017, Huang et al., 2019, or Zhang et al., 2021), with FIG. 7E providing a comparison of the false positive rate (with 10% or 5% cutoffs) on non-m⁵C sites between the different noted methods, while the reported methods showed false positives, no false positives were detected using the methods described herein, and FIG. 7F providing comparison of the m⁵C fractions detected by the different methods, methods provided herein detected modification fractions with over 95% for the two known m5C sites similar to canonical-BS treatments (e.g., Zymo kit), while all the other reported methods detected lower m⁵C fractions, suggesting these method may generate false negatives.

FIG. 8 shows how the BS recipes and methods disclosed herein create less bias in RNA degradation and show more uniform coverage in highly structured regions when compared to different previously disclosed methods (e.g., Zymo EZ RNA Methylation™ kit (“Zymo Kit”), Yang et al., 2017, Huang et al., 2019, or Zhang et al., 2021). Using 28s rRNA as an example, compositions and methods of previous studies were found to be more biased in degradation of high GC regions, particularly, the coverage near m⁵C sites (green dashed vertical lines) was found to have dropped, which can create bias in m⁵C stichometry.

FIGS. 9A-9D show results from detection of m⁵C sites in tRNA. FIG. 9A (from FIG. 1 of A. G. Torres et al., 2014, Trends in Molecular Medicine.) shows canonical tRNA modifications, with msC modifications at site 48, 49 and 50 being installed by NSUN2, while msC at site 38 is installed by DNMT2. FIGS. 9B-9D display msC sequencing analysis results that showed that all the detected msC fractions at site 48, 49 and 50 were sensitive to NSUN2 knockdown; whereas in contrast, m⁵C fraction at site 38 remained unchanged.

FIGS. 10A-10B show the results of detection and quantification of m⁵C sites in tRNA. FIG. 10A shows modification fractions at m⁵C sites detected in tRNA, most of m⁵C sites detected in tRNA showed high modification fractions. FIG. 10B shows three m⁵C sites detected in tRNA Gly^{CCC}. Two sites (49 and 50) showed very high m⁵C fraction, while one site (48) showed relatively lower fractions, while all the other C sites showed very low background.

FIGS. 11A-11B shows m⁵C sites distribution among many RNA species within HeLa cell total RNA. FIG. 11A shows the detected m⁵C sites distribution among different RNA species, while FIG. 11B shows m⁵C sites distribution within mRNA.

FIGS. 12A-12B shows m⁵C site detection in HeLa mRNA using R-1G recipe at condition D.5, when compared to those reported in the literature. More m⁵C sites were detected by the immediate method (˜1,241 sites), and these sites covered the majority of the sites reported in the literature. FIG. 12A shows the overlap with Huang et al., 2019¹⁵, while FIG. 12B shows the overlap with Zhang et al., 2021²¹.

FIG. 13 shows the distribution of modification level of m⁵C sites in HeLa cell mRNA. For the detected m⁵C sites in HeLa cell mRNA, the modification ratio differed among different sites, with about half of the sites displaying a more than 10% modified ratio.

FIGS. 14A-14B shows the number of m⁵C sites detected per gene, and gene ontology (GO) based functional annotations. FIG. 14A shows that of the modified genes identified, most carried only one m⁵C site. FIG. 15B shows that genes modified by m⁵C were found to be involved in various gene functions, include glycoprotein metabolism, cytoskeleton organization, cellular localization, etc.

FIGS. 15A-15B shows that m⁵C modification levels were consistent between different biological samples. FIG. 15A shows that overall modification level of m⁵C sites were consistent between HeLa (X axis) and HEK293T (Y axis) cell lines, while there are some differential modified sites. FIG. 15B shows that m⁵C site motifs in HeLa cell (top) were more G-rich (e.g., CGGGG (SEQ ID NO: 10), a signature associated with NSUN2, while HEK293T (bottom) m⁵C sites were CUCCA (SEQ ID NO: 11) motif enriched, which is a signature of NSUN6.

FIG. 16 show m⁵C sites detected in NSUN2 (X axis) or NSUN6 (Y axis) knockdown in HeLa cell line mRNA extracts. More than ˜90% of the modification fractions dropped in NSUN2 knockdown cell extracts, results which suggest that NSUN2 may play a major role in m⁵C modification in HeLa cells.

FIG. 17 shows the distribution of m⁵C site positions in the transcripts of modified genes from HeLa and HEK293T cells. m⁵C modifications were found to be enriched at the 5′-end of the transcripts (e.g., gene start and/or transcription (tx) start), indicating that m⁵C modification may be relevant to transcript translation.

FIGS. 18A-18B show m⁵C modification at the 5′-end of transcripts can modulate translation efficiency. FIG. 18A shows that compared to non-methylated gene, genes that showed methylation signal at their 5′-end were also more enriched for ribosomal density signal at the 5′-UTR of the transcripts (p values=1.05×10⁻⁶), while genes that showed methylation signal at 3′ end did not show significant enrichment signal of ribosome density signal (p=0.37). FIG. 18B shows that within CDS regions, both 5′-end and 3′-end methylated genes did not show ribosome density enrichment signal.

FIGS. 19A-19B show comparison of R-1G recipe and A7 recipe using analysis of DNA oligonucleotide AGCGA (SEQ ID NO: 3). FIG. 19A shows that, using R-1G to treat the model DNA oligo, it took 5 min at 98° C. to fully convert C to U-BS. Subsequent alkaline treatment converted U-BS adduct to U. FIG. 19B shows that, using A7 to treat the model DNA oligo, it took only 3 min at 98° C. to fully convert C to U-BS.

FIG. 20 shows Maldi-TOF MS monitoring of 5mC reaction with BS at 98° C. for different lengths of time. Only minimal reaction was detected after 20 minutes of incubation.

FIG. 21 shows Sanger sequencing of an 82mer synthetic DNA oligonucleotide containing both C and 5mC (SEQ ID NO: 8). Sanger sequencing showed that at least 8 min incubation was needed to complete C-to-U conversion while 5mC remained read as C even after 12 min incubation.

FIGS. 22A-22B shows how, in contrast to canonical-BS treatments (e.g., Zymo-BS treated), BS treatments disclosed herein (e.g., A7-BS) quantitatively deaminated 4mC. FIG. 22A depicts Maldi TOF MS results that showed that 4mC residue in (TA4mCTT (SEQ ID NO: 9) was not deaminated by canonical-BS treatment, but that DNA BS treatment disclosed herein quantitatively deaminated 4mC. FIG. 22B depicts Sanger sequencing data showing that two 4mC known sites in a 100 bp synthetic oligonucleotide (SEQ ID NO: 12) were exclusively read as T when utilizing DNA BS treatments disclosed herein, conversely when utilizing canonical-BS treatment, the two 4mC sites were both partially read as C. In both conditions, a 5mC site was read as C.

FIGS. 23A-23B shows a comparison of the DNA damage caused using canonical-BS treatments (e.g., “Zymo kit) and the disclosed BS recipe A7. Using fish gDNA (FIG. 23A), it was found that in all cases using A7 between 4 to 12 min, the degradation of fish gDNA was less than that caused by using canonical-BS treatments. The lower band represents smaller DNA fragments. Compared with A7, the Zymo kit treatment generated more lower bands and less higher bands, suggesting that it caused more DNA damage. Using synthetic 164 bp DNA (FIG. 23B), it was found that the Zymo kit again caused more DNA degradation than A7 between 4 to 12 min.

FIGS. 24A-24E show bisulfite conversion rates of DNA using recipes disclosed herein (e.g., A7) with various times compared to canonical-BS treatments (e.g., Zymo kit conditions). The results showed that not only is the background of the disclosed protocols much lower than when using Zymo kit conditions, but also that the range of the background is much lower as well. Use of recipe A7 with incubation of 10 minutes provided results characteristic of improvements described in this disclosure. FIG. 24A shows the average ratio of background noise calculated from FIG. 24B, which shows the raw background noise of different C sites along lambda DNA (SEQ ID NO: 15). FIG. 24C shows comparison of unconverted ratio for lambda DNA treated using the Zymo DNA methylation gold kit or the disclosed A7 recipe at various incubation times. The yellow (top) number represents the median unconverted rate while the red (bottom) number represents the average unconverted rate. FIG. 24D shows bisulfite conversion efficiency of lambda DNA treated with Zymo DNA methylation gold kit or the disclosed A7 recipe at various incubation times. FIG. 24E shows comparison of background from lambda DNA treated with Zymo DNA methylation gold kit or the disclosed A7 recipe at various incubation times. The results demonstrated that use of A7 results in much lower background and reduced range of background compared with Zymo kit treatment.

FIGS. 25A-25D show efficacy of recipes and protocols disclosed herein (“new-BS”) for 5mC analysis of low input DNA samples. 10 ng and 3.3 ng mESC starting gDNA was utilized to test the efficacy of the new-BS protocol (e.g., 98° C. for 10 min with recipe A7). The background noise and detection signal of 5mC after canonical-BS treatment (e.g., Zymo EZ DNA Methylation-Gold® Kit) or treatment with a protocol of the present disclosure was determined using spike-in 164mer dsDNA oligos (SEQ ID NO: 13; and anti-sense SEQ ID NO: 14). FIG. 25A shows the background and detected 5mC signals using canonical-BS treatment using 10 ng and 3 ng mES starting gDNA including spike in-oligos. FIG. 25B depicts a graphical analysis of the data presented in FIG. 25A, showing that new-BS treatments result in significantly lower background levels (% unconverted C) when compared to canonical-BS treatments. FIG. 26C shows the background and detected 5mC signals using BS treatment protocols of the immediate disclosure (e.g., 98° C. for 10 min with recipe A7) using 10 ng and 3 ng mES starting gDNA including spike in-oligos. FIG. 25D depicts a graphical analysis of the data presented in FIG. 25C, showing that new-BS treatment results in similar undesired 5mC conversion as canonical-BS treatments. As shown, canonical-BS treatments resulted in high background noise in low input DNA samples, and the noise increased when the input amount decreased. This noise can hinder the application of BS-seq in ultralow input samples.

FIG. 26 shows a comparison of the methylation level between canonical-BS treatments (Y axis) and BS protocols of the immediate disclosure (“new-BS”, X axis) in mESC gDNA (e.g., as described in FIGS. 25A-25D). Methylation level reported from data from canonical-BS treatments showed higher ratios than data reported from new-BS treatments of the immediate disclosure. This result may be due to the relatively high levels of background noise (e.g., insufficient conversion) associated with canonical-BS treatment.

FIG. 27 shows that canonical-BS treatment data reported more non-CpG sites than BS protocols of the present disclosure (e.g., as described in FIGS. 25A-25D). This observation may be due to relative increases in background noise in canonical-BS treatments when compared to BS protocols of the immediate disclosure. Background noise are random signal and more chance to be non-CpG sites, and can potentially cause problems in studying non-CpG methylation, leading to erroneous conclusions in biological studies.

FIGS. 28A-28B show coverage and conversion efficiency of BS treatments disclosed herein for mESC genomic regions with diverse GC contents. FIG. 28A shows that the coverage of genomic regions with diverse GC contents are similar between BS treatments of the immediate disclosure (“new-BS” as described in FIGS. 25A-25D) and canonical-BS treatments. FIG. 28B shows that the unconverted C ratio increases when the GC % of genomic regions increase, but that the unconversion ratios in all GC content regions showed lower background in BS treatments of disclosed herein when compared to canonical-BS treatments.

FIGS. 29A-29B show that BS protocols disclosed herein showed more evenly distributed genomic coverage in mESC gDNA when compared to canonical-BS treatments (e.g., as described in FIGS. 25A-25D). FIG. 29A shows the relative coverage (Z-score) of different genomic windows at a 100 kb overview, the distribution of BS protocols disclosed herein was narrower than the canonical-BS treatment data as shown with a statistical data in presented in a boxplot, interquartile range (IQR) was utilized to represent the statistical variance of the data, a comparison of canonical-BS treatments compared to BS protocols described herein showed a 7.5% and 9.9% decrease of IQR value for 10 ng and 3.3 ng samples respectively. FIG. 29B shows the raw genomic coverage data for all of the mESC chromosomes.

FIG. 30 shows a comparison of the percentage unconverted C (background) in lambda DNA spiked into gDNA from 1, 10, or 100 mESCs, where the DNA has been subjected to canonical-BS treatments or BS treatments disclosed herein (“new-BS”).

FIG. 31 shows a comparison of the percentage unconverted C (background) in mitochondrial DNA from gDNA extracts from 1, 10, or 100 mESCs, where the DNA has been subjected to canonical-BS treatments or BS treatments disclosed herein (“new-BS”).

FIG. 32A shows a Maldi-TOF MS demonstrating that 5hmC was converted to CMS within 1 min using A7 treatment of oligonucleotide AG5hmCGA (SEQ ID NO: 5) at 98° C.

FIG. 32B shows a diagram of the process of 5hmC to CMS conversion.

FIG. 33A shows a Maldi-TOF MS demonstrating that 5fC was converted to U-BS within 30 min at 98° C. using A7 treatment of oligonucleotide AG5fCGA (SEQ ID NO: 6).

FIG. 33B shows a diagram of the process of 5fC to U conversion.

FIG. 34A shows a Maldi-TOF MS demonstrating that 5caC was converted to U-BS within 3 min at 98° C.

FIG. 34B shows a diagram of the process of 5caC to U conversion.

FIG. 35 shows Maldi-TOF MS results demonstrating that APOBEC3A efficiently deaminated 5mC to T, while CMS resisted deamination and was kept intact upon APOBEC3A treatment.

FIG. 36 shows Sanger sequencing results demonstrating that 5mC was quantitatively converted to T, and 5hmC was converted to 5hmU mostly and thus read as T, although a small portion of 5hmC was not deaminated. In contrast, CMS resisted the deamination upon APOBEC3A treatment and thus was still read as C.

FIG. 37 shows a schematic of a workflow for sequencing 5mC and 5hmC in DNA using the disclosed methods. Genomic DNA contains C and its derivatives such as 5mC, 5hmC, 5fC and 5caC. After treatment with the disclosed BS reagents and conditions, C, 5fC and 5caC are converted to U, 5hmC is converted to CMS, and 5mC remains intact. One half of the sample (left) proceeds to sequencing where only 5mC and 5hmC sites are read as C. The other half of the sample (right) is treated with APOBEC3A to convert 5mC to T while keeping CMS intact. After sequencing, only original 5hmC sites will be read as C while all the other C derivatives will be read as T. Thus, 5hmC sites are determined. The subtraction of the two libraries gives the original 5mC sites.

### DETAILED DESCRIPTION

Aspects of the present disclosure relate to compositions, methods, and kits for detection and analysis of methylated DNA and methylated RNA. Certain aspects are directed to compositions for bisulfite treatment of methylated DNA and methylated RNA, including bisulfite solutions that do not comprise sodium bisulfite. Also disclosed, in some aspects, are methods for bisulfite treatment of methylated DNA and methylated RNA, including methods comprising incubation for short time periods (e.g., <15 minutes) at high temperatures (e.g., >95° C.) using the disclosed bisulfite solutions. Kits including the disclosed compositions are also described herein, along with instructions for analysis of methylated DNA and/or methylated RNA. Aspects of the disclosure provide bisulfite sequencing methods comprising rapid bisulfite treatment, low background noise, and high sensitivity, enabling highly accurate sequencing of m⁵C in RNA and 5mC in DNA starting from low-input biological RNA or DNA samples.

### I. DNA Processing Methods

Aspects of the present disclosure relate to compositions and methods for DNA processing. Particular aspects relate to compositions comprising ammonium bisulfite and methods for use of such compositions in bisulfite treatment of DNA. Accordingly, disclosed herein, in some aspects, are methods for DNA processing comprising incubating a solution comprising a DNA molecule and ammonium bisulfite under conditions sufficient to deaminate a cytosine residue of the DNA molecule, where the solution does not comprise sodium bisulfite or added sodium bisulfite. Such methods may further comprise subjecting the DNA molecule to alkaline (i.e., basic) conditions. As disclosed herein, incubating one or more DNA molecules in a bisulfite solution of the disclosure under appropriate conditions results in extremely rapid deamination of cytosines with low DNA degradation while preserving 5-methylcytosine (5mC), leading to identification of methylated nucleotides with very low false positive rate. In some aspects, methods provided herein provide BS treatments suitable for accurately distinguishing 5mC from N4-methylcytosine (4mC). In some aspects, methods provided herein facilitate deamination of 4mC at greater rate relative to canonical-BS treatments. In some aspects, methods provided herein facilitate conversion of 4mC to uracil at greater rate relative to canonical-BS treatments. In some aspects methods provided herein quantitatively deaminates 4mC. In some aspects, methods provided herein facilitate deamination of 4mC at an efficiency of greater than about or equal to about 50%, 55%, 60%, 65%, 70%, 75%, 80%, 85%, 90%, 95%, 98%, 99%, or 100%, or any range derivable therein. In some aspects, methods provided herein substantially avoid BS treatment false positives generated by the existence of 4mC in the genome.

In some aspects, DNA processing methods of the disclosure include incubating one or more DNA molecules in a bisulfite solution, where the bisulfite solution comprises ammonium bisulfite and does not comprise sodium bisulfite or added sodium bisulfite. In some aspects, the bisulfite solution comprises sodium at a concentration of, or of at most 1 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M or less. In some aspects, the solution does not comprise sodium. In some aspects, the solution comprises ammonium sulfite at a concentration of, or of at most 10 M, 1 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, or less. In certain aspects the solution does not comprise ammonium sulfite or added ammonium sulfite.

In certain aspects a solution (e.g., bisulfite solution) of the disclosure comprises between 50% and 70% ammonium bisulfite by weight, including any range or value derivable therein. In some aspects, the solution comprises at least, at most, or about 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, or 70% ammonium bisulfite by weight, or any range or value derivable therein. In some aspects, the solution comprises at least, at most, or about 66%, 66.01%, 66.02%, 66.03%, 66.04%, 66.05%, 66.06%, 66.07%, 66.08%, 66.09%, 66.1%, 66.11%, 66.12%, 66.13%, 66.14%, 66.15%, 66.16%, 66.17%, 66.18%, 66.19%, 66.2%, 66.21%, 66.22%, 66.23%, 66.24%, 66.25%, 66.26%, 66.27%, 66.28%, 66.29%, 66.3%, 66.31%, 66.32%, 66.33%, 66.34%, 66.35%, 66.36%, 66.37%, 66.38%, 66.39%, 66.4%, 66.41%, 66.42%, 66.43%, 66.44%, 66.45%, 66.46%, 66.47%, 66.48%, 66.49%, 66.5%, 66.51%, 66.52%, 66.53%, 66.54%, 66.55%, 66.56%, 66.57%, 66.58%, 66.59%, 66.6%, 66.61%, 66.62%, 66.63%, 66.64%, 66.65%, 66.66%, 66.67%, 66.68%, 66.69%, 66.7%, 66.71%, 66.72%, 66.73%, 66.74%, 66.75%, 66.76%, 66.77%, 66.78%, 66.79%, 66.8%, 66.81%, 66.82%, 66.83%, 66.84%, 66.85%, 66.86%, 66.87%, 66.88%, 66.89%, 66.9%, 66.91%, 66.92%, 66.93%, 66.94%, 66.95%, 66.96%, 66.97%, 66.98%, 66.99%, or 67% ammonium bisulfite by weight, or any range or value derivable therein. In some aspects, the solution comprises about 66.67% ammonium bisulfite by weight. In some aspects, a bisulfite solution does not comprise ammonium sulfite or added ammonium sulfite. In some aspects, a bisulfite solution comprises ammonium sulfite.

In some aspects, the bisulfite solution is at a bisulfite concentration of between 6.5 M and 10 M, including any range or value derivable therein. In some aspects, the bisulfite solution is at a bisulfite concentration of at least, at most, or about 6.5 M, 6.6 M, 6.7 M, 6.8 M, 6.9 M, 7 M, 7.1 M, 7.2 M, 7.3 M, 7.4 M, 7.5 M, 7.6 M, 7.7 M, 7.8 M, 7.9 M, 8 M, 8.1 M, 8.2 M, 8.3 M, 8.4 M, 8.5 M, 8.6 M, 8.7 M, 8.8 M, 8.9 M, 9 M, 9.1 M, 9.2 M, 9.3 M, 9.4 M, 9.5 M, 9.6 M, 9.7 M, 9.8 M, 9.9 M, or 10 M, or any range or value derivable therein. In some aspects, the bisulfite solution is at a bisulfite concentration of about 9.5 M. In some aspects, the bisulfite solution is at a bisulfite concentration of 9.5 M.

A bisulfite solution of the disclosure may be generated, for example, by mixing two ammonium bisulfite solutions having different % ammonium bisulfite by weight. For example, a bisulfite solution of the disclosure may be generated by mixing a 70% ammonium bisulfite solution and a 50% ammonium bisulfite solution. In some aspects, a 70% ammonium bisulfite solution and a 50% ammonium bisulfite solution are mixed at a ratio of, for example, 10:0.1, 10:0.2, 10:0.3, 10:0.4, 10:0.5, 10:0.6, 10:0.7, 10:0.8. 10:0.9, 10:1, 10:1.1, 10:1.2, 10:1.3, 10:1.4, 10:1.5, 10:1.6, 10:1.7, 10:1.8, 10:1.9, or 10:2, or any range or value derivable therein. In some aspects, a 70% ammonium bisulfite solution and a 50% ammonium bisulfite solution are mixed at a ratio of 10:1.

In some aspects, a DNA processing method comprises incubating one or more DNA molecules in a bisulfite solution of the disclosure (e.g., a solution comprising ammonium bisulfite, such as 50%-70% ammonium bisulfite, which does not comprise sodium bisulfite) at a temperature of at least 80° C. for at most 20 minutes. In some aspects, the method comprises incubating one or more DNA molecules in a bisulfite solution at a temperature of at least, at most, or about 80° C., 80.1° C., 80.2° C., 80.3° C., 80.4° C., 80.5° C., 80.6° C., 80.7° C., 80.8° C., 80.9° C., 81° C., 81.1° C., 81.2° C., 81.3° C., 81.4° C., 81.5° C., 81.6° C., 81.7° C., 81.8° C., 81.9° C., 82° C., 82.1° C., 82.2° C., 82.3° C., 82.4° C., 82.5° C., 82.6° C., 82.7° C., 82.8° C., 82.9° C., 83° C., 83.1° C., 83.2° C., 83.3° C., 83.4° C., 83.5° C., 83.6° C., 83.7° C., 83.8° C., 83.9° C., 84° C., 84.1° C., 84.2° C., 84.3° C., 84.4° C., 84.5° C., 84.6° C., 84.7° C., 84.8° C., 84.9° C., 85° C., 85.1° C., 85.2° C., 85.3° C., 85.4° C., 85.5° C., 85.6° C., 85.7° C., 85.8° C., 85.9° C., 86° C., 86.1° C., 86.2° C., 86.3° C., 86.4° C., 86.5° C., 86.6° C., 86.7° C., 86.8° C., 86.9° C., 87° C., 87.1° C., 87.2° C., 87.3° C., 87.4° C., 87.5° C., 87.6° C., 87.7° C., 87.8° C., 87.9° C., 88° C., 88.1° C., 88.2° C., 88.3° C., 88.4° C., 88.5° C., 88.6° C., 88.7° C., 88.8° C., 88.9° C., 89° C., 89.1° C., 89.2° C., 89.3° C., 89.4° C., 89.5° C., 89.6° C., 89.7° C., 89.8° C., 89.9° C., 90° C., 90.1° C., 90.2° C., 90.3° C., 90.4° C., 90.5° C., 90.6° C., 90.7° C., 90.8° C., 90.9° C., 91° C., 91.1° C., 91.2° C., 91.3° C., 91.4° C., 91.5° C., 91.6° C., 91.7° C., 91.8° C., 91.9° C., 92° C., 92.1° C., 92.2° C., 92.3° C., 92.4° C., 92.5° C., 92.6° C., 92.7° C., 92.8° C., 92.9° C., 93° C., 93.1° C., 93.2° C., 93.3° C., 93.4° C., 93.5° C., 93.6° C., 93.7° C., 93.8° C., 93.9° C., 94° C., 94.1° C., 94.2° C., 94.3° C., 94.4° C., 94.5° C., 94.6° C., 94.7° C., 94.8° C., 94.9° C., 95° C., 95.1° C., 95.2° C., 95.3° C., 95.4° C., 95.5° C., 95.6° C., 95.7° C., 95.8° C., 95.9° C., 96° C., 96.1° C., 96.2° C., 96.3° C., 96.4° C., 96.5° C., 96.6° C., 96.7° C., 96.8° C., 96.9° C., 97° C., 97.1° C., 97.2° C., 97.3° C., 97.4° C., 97.5° C., 97.6° C., 97.7° C., 97.8° C., 97.9° C., 98° C., 98.1° C., 98.2° C., 98.3° C., 98.4° C., 98.5° C., 98.6° C., 98.7° C., 98.8° C., 98.9° C., 99° C., 99.1° C., 99.2° C., 99.3° C., 99.4° C., 99.5° C., 99.6° C., 99.7° C., 99.8° C., 99.9° C. (or any range or value derivable therein) for at most or about 15, 14.9, 14.8, 14.7, 14.6, 14.5, 14.4, 14.3, 14.2, 14.1, 14, 13.9, 13.8, 13.7, 13.6, 13.5, 13.4, 13.3, 13.2, 13.1, 13, 12.9, 12.8, 12.7, 12.6, 12.5, 12.4, 12.3, 12.2, 12.1, 12, 11.9, 11.8, 11.7, 11.6, 11.5, 11.4, 11.3, 11.2, 11.1, 11, 10.9, 10.8, 10.7, 10.6, 10.5, 10.4, 10.3, 10.2, 10.1, 10, 9.9, 9.8, 9.7, 9.6, 9.5, 9.4, 9.3, 9.2, 9.1, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 minutes (or any range or value derivable therein). Any combination of the preceding incubation times and temperatures may be used in a DNA processing method of the present disclosure.

In some aspects, a DNA processing method comprises incubating one or more DNA molecules in a bisulfite solution of the disclosure at a temperature of at least 95° C. for at most 12 minutes, at a temperature of at least 96° C. for at most 12 minutes, at a temperature of at least 97° C. for at most 12 minutes, at a temperature of at least 98° C. for at most 12 minutes, at a temperature of at least 99° C. for at most 12 minutes, at a temperature of at least 95° C. for at most 11 minutes, at a temperature of at least 96° C. for at most 11 minutes, at a temperature of at least 97° C. for at most 11 minutes, at a temperature of at least 98° C. for at most 11 minutes, at a temperature of at least 99° C. for at most 11 minutes, at a temperature of at least 95° C. for at most 10 minutes, at a temperature of at least 96° C. for at most 10 minutes, at a temperature of at least 97° C. for at most 10 minutes, at a temperature of at least 98° C. for at most 10 minutes, at a temperature of at least 99° C. for at most 10 minutes, at a temperature of at least 95° C. for at most 9 minutes, at a temperature of at least 96° C. for at most 9 minutes, at a temperature of at least 97° C. for at most 9 minutes, at a temperature of at least 98° C. for at most 9 minutes, at a temperature of at least 99° C. for at most 9 minutes, at a temperature of at least 95° C. for at most 8 minutes, at a temperature of at least 96° C. for at most 8 minutes, at a temperature of at least 97° C. for at most 8 minutes, at a temperature of at least 98° C. for at most 8 minutes, at a temperature of at least 99° C. for at most 8 minutes, at a temperature of at least 95° C. for at most 7 minutes, at a temperature of at least 96° C. for at most 7 minutes, at a temperature of at least 97° C. for at most 7 minutes, at a temperature of at least 98° C. for at most 7 minutes, at a temperature of at least 99° C. for at most 7 minutes, at a temperature of at least 95° C. for at most 6 minutes, at a temperature of at least 96° C. for at most 6 minutes, at a temperature of at least 97° C. for at most 6 minutes, at a temperature of at least 98° C. for at most 6 minutes, at a temperature of at least 99° C. for at most 6 minutes, at a temperature of at least 95° C. for at most 5 minutes, at a temperature of at least 96° C. for at most 5 minutes, at a temperature of at least 97° C. for at most 5 minutes, at a temperature of at least 98° C. for at most 5 minutes, or at a temperature of at least 99° C. for at most 5 minutes.

As disclosed herein, incubating DNA molecules with a bisulfite solution of the present disclosure (e.g., a solution comprising ammonium bisulfite such as 50%-70% ammonium bisulfite which does not comprise sodium bisulfite, or added sodium bisulfite) under appropriate conditions (e.g., at a temperature of at least 95° C. for at most 12 minutes) is sufficient to deaminate a majority of cytosine residues in the DNA molecules. In some aspects, after incubating DNA molecules with a bisulfite solution of the present disclosure under appropriate conditions, greater than 90% of the DNA molecules comprise no cytosine residue. In some aspects, greater than or equal to 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, or 99.9% (or any range or value derivable therein) of the DNA molecules comprise no cytosine residue. In some aspects, greater than 99% of the DNA molecules comprise no cytosine residue.

DNA processing methods of the disclosure may be useful in, for example, preparing DNA molecules for sequencing in order to detect, quantify, and/or analyze DNA cytosine methylation. In some aspects, DNA processing methods of the disclosure provide DNA molecules for sequencing analysis that result in a reduced level of false positives, increased level of true positives, reduced level of false negatives, and/or increased level of true negatives relative to canonical-BS treatments.

### II. RNA Processing Methods

Aspects of the present disclosure relate to compositions and methods for RNA processing. Particular aspects relate to compositions comprising ammonium bisulfite and methods for use of such compositions in bisulfite treatment of RNA. Accordingly, disclosed herein, in some aspects, are methods for RNA processing comprising incubating a solution comprising an RNA molecule, ammonium bisulfite, and ammonium sulfite under conditions sufficient to deaminate a cytosine residue of the RNA molecule, where the solution does not comprise sodium bisulfite or added sodium bisulfite. Such methods may further comprise subjecting the RNA molecule to alkaline (i.e., basic) conditions. As disclosed herein, incubating one or more RNA molecules in a bisulfite solution of the disclosure under appropriate conditions results in extremely rapid deamination of cytosines with low RNA degradation, leading to identification of methylated nucleotides with very low false positive rate. In some aspects, methods disclosed herein result in a reduced level of background noise (e.g., unconverted cytosines) relative to canonical-BS treatments.

In some aspects, RNA processing methods of the disclosure include incubating one or more RNA molecules in a bisulfite solution, where the bisulfite solution comprises ammonium bisulfite and ammonium sulfite, and where the bisulfite solution does not comprise sodium bisulfite, or added sodium bisulfite. In some aspects, the bisulfite solution comprises sodium at a concentration of, or of less than 1 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M or less. In some aspects, the bisulfite solution does not comprise sodium.

In certain aspects a solution (e.g., bisulfite solution) of the disclosure comprises between 50% and 70% ammonium bisulfite by weight, including any range or value derivable therein. In some aspects, the solution comprises at least, at most, or about 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, or 70% ammonium bisulfite by weight, or any range or value derivable therein. In some aspects, the solution comprises at least, at most, or about 66%, 66.01%, 66.02%, 66.03%, 66.04%, 66.05%, 66.06%, 66.07%, 66.08%, 66.09%, 66.1%, 66.11%, 66.12%, 66.13%, 66.14%, 66.15%, 66.16%, 66.17%, 66.18%, 66.19%, 66.2%, 66.21%, 66.22%, 66.23%, 66.24%, 66.25%, 66.26%, 66.27%, 66.28%, 66.29%, 66.3%, 66.31%, 66.32%, 66.33%, 66.34%, 66.35%, 66.36%, 66.37%, 66.38%, 66.39%, 66.4%, 66.41%, 66.42%, 66.43%, 66.44%, 66.45%, 66.46%, 66.47%, 66.48%, 66.49%, 66.5%, 66.51%, 66.52%, 66.53%, 66.54%, 66.55%, 66.56%, 66.57%, 66.58%, 66.59%, 66.6%, 66.61%, 66.62%, 66.63%, 66.64%, 66.65%, 66.66%, 66.67%, 66.68%, 66.69%, 66.7%, 66.71%, 66.72%, 66.73%, 66.74%, 66.75%, 66.76%, 66.77%, 66.78%, 66.79%, 66.8%, 66.81%, 66.82%, 66.83%, 66.84%, 66.85%, 66.86%, 66.87%, 66.88%, 66.89%, 66.9%, 66.91%, 66.92%, 66.93%, 66.94%, 66.95%, 66.96%, 66.97%, 66.98%, 66.99%, or 67% ammonium bisulfite by weight, or any range or value derivable therein.

In some aspects, the bisulfite solution is at a bisulfite concentration of between 6.5 M and 10 M, including any range or value derivable therein. In some aspects, the bisulfite solution is at a bisulfite concentration of at least, at most, or about 6.5 M, 6.6 M, 6.7 M, 6.8 M, 6.9 M, 7.0 M, 7.1 M, 7.2 M, 7.3 M, 7.4 M, 7.5 M, 7.6 M, 7.7 M, 7.8 M, 7.9 M, 8.0 M, 8.1 M, 8.2 M, 8.3 M, 8.4 M, 8.5 M, 8.6 M, 8.7 M, 8.8 M, 8.9 M, 9.0 M, 9.1 M, 9.2 M, 9.3 M, 9.4 M, 9.5 M, 9.6 M, 9.7 M, 9.8 M, 9.9 M, or 10 M, or any range or value derivable therein. In some aspects, the bisulfite solution is at a bisulfite concentration of about 7.0 M. In some aspects, the bisulfite solution is at a bisulfite concentration of 7.0 M.

In some aspects, a bisulfite solution of the disclosure used for RNA processing comprises between 5% and 15% ammonium sulfite by weight, or any range or value derivable therein. In some aspects, the solution comprises at least, at most, or about 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, or 15% ammonium sulfite by weight, or any range or value derivable therein. In some aspects, the bisulfite solution comprises between 8% and 12% ammonium sulfite by weight. In some aspects, the bisulfite solution comprises about 10% ammonium sulfite by weight. In some aspects, a bisulfite solution is generated by mixing an ammonium bisulfite solution (e.g., 50%-70% ammonium bisulfite) with ammonium sulfite (e.g., ammonium sulfite monohydrate solid).

In some aspects, an RNA processing method comprises incubating one or more RNA molecules in a bisulfite solution of the disclosure (e.g., a solution comprising ammonium bisulfite and ammonium sulfite which does not comprise sodium bisulfite, or added sodium bisulfite) at a temperature of at least 80° C. for at most 20 minutes. In some aspects, the method comprises incubating one or more RNA molecules in a bisulfite solution at a temperature of at least, at most, or about 80° C., 80.1° C., 80.2° C., 80.3° C., 80.4° C., 80.5° C., 80.6° C., 80.7° C., 80.8° C., 80.9° C., 81° C., 81.1° C., 81.2° C., 81.3° C., 81.4° C., 81.5° C., 81.6° C., 81.7° C., 81.8° C., 81.9° C., 82° C., 82.1° C., 82.2° C., 82.3° C., 82.4° C., 82.5° C., 82.6° C., 82.7° C., 82.8° C., 82.9° C., 83° C., 83.1° C., 83.2° C., 83.3° C., 83.4° C., 83.5° C., 83.6° C., 83.7° C., 83.8° C., 83.9° C., 84° C., 84.1° C., 84.2° C., 84.3° C., 84.4° C., 84.5° C., 84.6° C., 84.7° C., 84.8° C., 84.9° C., 85° C., 85.1° C., 85.2° C., 85.3° C., 85.4° C., 85.5° C., 85.6° C., 85.7° C., 85.8° C., 85.9° C., 86° C., 86.1° C., 86.2° C., 86.3° C., 86.4° C., 86.5° C., 86.6° C., 86.7° C., 86.8° C., 86.9° C., 87° C., 87.1° C., 87.2° C., 87.3° C., 87.4° C., 87.5° C., 87.6° C., 87.7° C., 87.8° C., 87.9° C., 88° C., 88.1° C., 88.2° C., 88.3° C., 88.4° C., 88.5° C., 88.6° C., 88.7° C., 88.8° C., 88.9° C., 89° C., 89.1° C., 89.2° C., 89.3° C., 89.4° C., 89.5° C., 89.6° C., 89.7° C., 89.8° C., 89.9° C., 90° C., 90.1° C., 90.2° C., 90.3° C., 90.4° C., 90.5° C., 90.6° C., 90.7° C., 90.8° C., 90.9° C., 91° C., 91.1° C., 91.2° C., 91.3° C., 91.4° C., 91.5° C., 91.6° C., 91.7° C., 91.8° C., 91.9° C., 92° C., 92.1° C., 92.2° C., 92.3° C., 92.4° C., 92.5° C., 92.6° C., 92.7° C., 92.8° C., 92.9° C., 93° C., 93.1° C., 93.2° C., 93.3° C., 93.4° C., 93.5° C., 93.6° C., 93.7° C., 93.8° C., 93.9° C., 94° C., 94.1° C., 94.2° C., 94.3° C., 94.4° C., 94.5° C., 94.6° C., 94.7° C., 94.8° C., 94.9° C., 95° C., 95.1° C., 95.2° C., 95.3° C., 95.4° C., 95.5° C., 95.6° C., 95.7° C., 95.8° C., 95.9° C., 96° C., 96.1° C., 96.2° C., 96.3° C., 96.4° C., 96.5° C., 96.6° C., 96.7° C., 96.8° C., 96.9° C., 97° C., 97.1° C., 97.2° C., 97.3° C., 97.4° C., 97.5° C., 97.6° C., 97.7° C., 97.8° C., 97.9° C., 98° C., 98.1° C., 98.2° C., 98.3° C., 98.4° C., 98.5° C., 98.6° C., 98.7° C., 98.8° C., 98.9° C., 99° C., 99.1° C., 99.2° C., 99.3° C., 99.4° C., 99.5° C., 99.6° C., 99.7° C., 99.8° C., or 99.9° C. (or any range or value derivable therein) for at most or about 15, 14.9, 14.8, 14.7, 14.6, 14.5, 14.4, 14.3, 14.2, 14.1, 14, 13.9, 13.8, 13.7, 13.6, 13.5, 13.4, 13.3, 13.2, 13.1, 13, 12.9, 12.8, 12.7, 12.6, 12.5, 12.4, 12.3, 12.2, 12.1, 12, 11.9, 11.8, 11.7, 11.6, 11.5, 11.4, 11.3, 11.2, 11.1, 11, 10.9, 10.8, 10.7, 10.6, 10.5, 10.4, 10.3, 10.2, 10.1, 10, 9.9, 9.8, 9.7, 9.6, 9.5, 9.4, 9.3, 9.2, 9.1, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 minutes (or any range or value derivable therein). Any combination of the preceding incubation times and temperatures may be used in an RNA processing method of the present disclosure.

In some aspects, an RNA processing method comprises incubating one or more RNA molecules in a bisulfite solution of the disclosure at a temperature of at least 95° C. for at most 12 minutes, at a temperature of at least 96° C. for at most 12 minutes, at a temperature of at least 97° C. for at most 12 minutes, at a temperature of at least 98° C. for at most 12 minutes, at a temperature of at least 99° C. for at most 12 minutes, at a temperature of at least 95° C. for at most 11 minutes, at a temperature of at least 96° C. for at most 11 minutes, at a temperature of at least 97° C. for at most 11 minutes, at a temperature of at least 98° C. for at most 11 minutes, at a temperature of at least 99° C. for at most 11 minutes, at a temperature of at least 95° C. for at most 10 minutes, at a temperature of at least 96° C. for at most 10 minutes, at a temperature of at least 97° C. for at most 10 minutes, at a temperature of at least 98° C. for at most 10 minutes, at a temperature of at least 99° C. for at most 10 minutes, at a temperature of at least 95° C. for at most 9 minutes, at a temperature of at least 96° C. for at most 9 minutes, at a temperature of at least 97° C. for at most 9 minutes, at a temperature of at least 98° C. for at most 9 minutes, at a temperature of at least 99° C. for at most 9 minutes, at a temperature of at least 95° C. for at most 8 minutes, at a temperature of at least 96° C. for at most 8 minutes, at a temperature of at least 97° C. for at most 8 minutes, at a temperature of at least 98° C. for at most 8 minutes, at a temperature of at least 99° C. for at most 8 minutes, at a temperature of at least 95° C. for at most 7 minutes, at a temperature of at least 96° C. for at most 7 minutes, at a temperature of at least 97° C. for at most 7 minutes, at a temperature of at least 98° C. for at most 7 minutes, at a temperature of at least 99° C. for at most 7 minutes, at a temperature of at least 95° C. for at most 6 minutes, at a temperature of at least 96° C. for at most 6 minutes, at a temperature of at least 97° C. for at most 6 minutes, at a temperature of at least 98° C. for at most 6 minutes, at a temperature of at least 99° C. for at most 6 minutes, at a temperature of at least 95° C. for at most 5 minutes, at a temperature of at least 96° C. for at most 5 minutes, at a temperature of at least 97° C. for at most 5 minutes, at a temperature of at least 98° C. for at most 5 minutes, or at a temperature of at least 99° C. for at most 5 minutes.

As disclosed herein, incubating RNA molecules with a bisulfite solution of the present disclosure (e.g., a solution comprising ammonium bisulfite and ammonium sulfite which does not comprise sodium bisulfite, or added sodium bisulfite) under appropriate conditions (e.g., at a temperature of at least 95° C. for at most 12 minutes) is sufficient to deaminate a majority of cytosine residues in the RNA molecules. In some aspects, after incubating RNA molecules with a bisulfite solution of the present disclosure under appropriate conditions, greater than 90% of the RNA molecules comprise no cytosine residue. In some aspects, greater than 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, or 99.9% (or any range or value derivable therein) of the RNA molecules comprise no cytosine residue. In some aspects, greater than 99% of the RNA molecules comprise no cytosine residue.

RNA processing methods of the disclosure may be useful in, for example, preparing RNA molecules for sequencing in order to detect, quantify, and/or analyze RNA cytosine methylation.

### III. 5hmC Analysis Methods

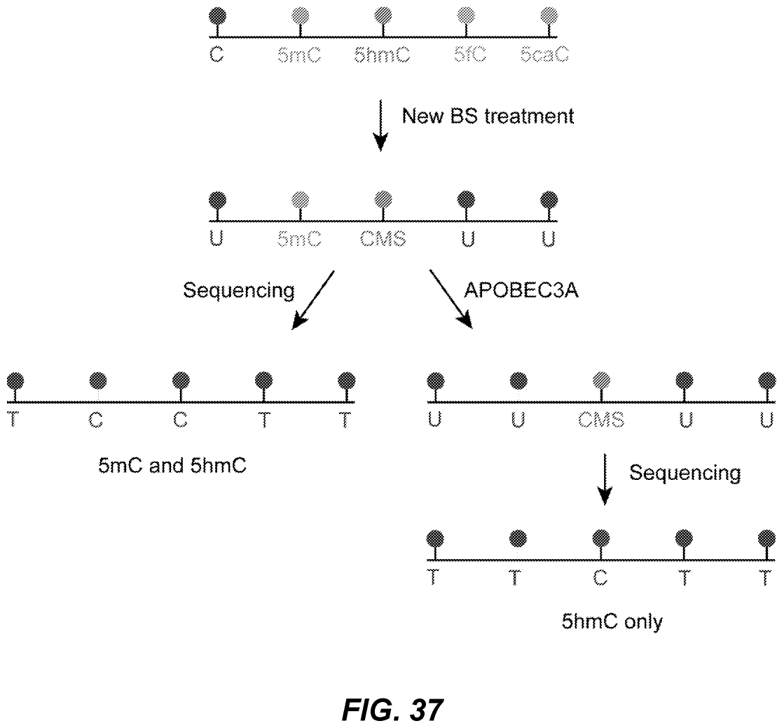

Aspects of the present disclosure relate to compositions and methods for detection, quantification, and analysis of 5-hydroxymethylcytosine (5hmC) in DNA. As described herein, the disclosed DNA processing methods are useful in rapid deamination of cytosine, and also in rapid spontaneous conversion of 5hmC to cytosine methylene sulfonate (CMS). APOBEC3A has been reported to have high deamination reactivity on C and 5mC²⁸. Accordingly, disclosed herein, in certain aspects, are methods for 5hmC analysis comprising incubating DNA molecules in a bisulfite solution of the disclosure (e.g., a solution comprising ammonium bisulfite such as 50%-70% ammonium bisulfite which does not comprise sodium bisulfite, or added sodium bisulfite) under sufficient conditions (e.g., at a temperature of at least 95° C. for at most 12 minutes), followed by subjecting the DNA molecules to alkaline conditions, thereby converting Cs to Us and 5hmCs to CMSs. Following this, in some cases, a portion of the DNA molecules are treated with an APOBEC deaminase enzyme (e.g., APOBEC3A under appropriate conditions such as those disclosed in Schutsky, E., DeNizio, et al. Nat Biotechnol 36, 1083-1090 (2018), incorporated herein by reference in its entirety), thus converting 5mCs to Us. After this, all the DNA molecules are subjected to sequencing and the sequences compared to identify 5hmC residues on the original DNA molecules. A schematic diagram of an example of a 5hmC analysis method of the disclosure is provided in FIG. 37.

### IV. General Assay Methods

### A. Detection and Analysis of Methylated Nucleic Acid

Aspects of the methods include assaying nucleic acids to determine expression levels and/or methylation levels of nucleic acids (e.g., DNA, RNA). Certain example methods for detection and analysis of nucleic acid methylation are described herein.

In certain aspects, methods provided herein facilitate generation of BS-treated sequencing libraries using low and/or ultralow DNA inputs. In certain aspects, methods provided herein facilitate generation of BS-treated sequencing libraries using low and/or ultralow RNA inputs. In some aspects, methods provided herein reduce levels of background in assays comprising low and/or ultralow DNA inputs relative to canonical-BS treatments. In some aspects, methods provided herein reduce levels of background in assays comprising low and/or ultralow RNA inputs relative to canonical-BS treatments. In some aspects, methods provided herein reduce false positive rates by equal to about or greater than about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fold, or any range derivable therein, when compared to canonical-BS treatments. In some aspects, methods provided herein increase the rate of true positive detection by equal to about or greater than about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or any range derivable therein, when compared to canonical-BS treatments.

In some aspects, methods provided herein reduce the rate of unconverted C in high GC % regions relative to canonical-BS treatments. In some aspects, methods provided herein reduce the rate of unconverted C in high GC % regions by equal to about or greater than about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or any range derivable therein, when compared to canonical-BS treatments.

### 1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5mC) present in a hydrolyzed DNA sample. The method includes hydrolyzing the DNA into its constituent nucleoside bases, the 5mC and dC bases are separated chromatographically and, then, the fractions are measured. Then, the 5mC/dC ratio can be calculated for each sample, and this can be compared between the experimental and control samples.

### 2. LC-MS/MS

Liquid chromatography coupled with tandem mass spectrometry (LC-MS/MS) is an high-sensitivity approach to HPLC-UV, which requires much smaller quantities of the hydrolyzed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled.

### 3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay—LINE-1, available from Active Motif; 5-mC DNA ELISA Kit, available from Zymo Research; MethylFlash Methylated DNA5-mC Quantification Kit and MethylFlash Methylated DNA5-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody; and then (3) colorimetric/fluorometric detection reagents.

The Global DNA Methylation Assay LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels.

### 4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ˜5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

### 5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

### 6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme MspI, which will cut at all CCGG sites. The enzyme EcoRI is included in both reactions as an internal control. Both MspI and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/MspI ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

### 7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5-methylcytosine (5mC) residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5mC-enriched fraction of the genome is not only a less expensive approach, but it also allows one to increase the sequencing coverage and, therefore, precision in revealing differentially-methylated regions. Sequencing could be done using any existing NGS platform; Illumina™ and Life Technologies™ both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

8. Methods that Exclude Bisulfite Conversion

In some aspects, direct detection of modified bases without bisulfite conversion may be used to detect methylation. Pacific Biosciences company has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include nanopore-based single-molecule real-time sequencing technology (SMRT), which is able to detect modified bases directly (described in Laszlo A. H. et al., Proc. Natl. Acad. Sci. USA. 2013 and Schreiber J., et al., Proc. Natl. Acad. Sci. USA. 2013, which are herein incorporated by reference).

### 9. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent®), the GeneChip Human Promoter 1.0R Array and the GeneChip Human Tiling 2.0R Array Set (Affymetrix®).

The search for differentially-methylated regions using bisulfite-converted DNA could be done with the use of different techniques. Some of them are easier to perform and analyze than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina™, can be used. The arrays can be used to detect methylation status of genes, including miRNA promoters, 5′ UTR, 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina™, 96 or 384 user-specified CpG loci are analysed with the GoldenGate® Assay for Methylation. Differently from the BeadChip assay, the VeraCode assay requires the BeadXpress® Reader for scanning.

### 10. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. Moreover, coverage in NGS could be good enough to quantify the methylation level for particular loci. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then, NGS and mapping to the genome are performed. The number of reads for each HpaII site correlates with its methylation level.

Recently, a number of restriction enzymes have been discovered that use methylated DNA as a substrate (methylation-dependent endonucleases). These include, for example: BisI, BlsI, GlaI, GluI, KroI, MteI, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other.

### B. Sequencing

### 1. DNA Sequencing

In some aspects, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some aspects, a unique genetic readout for each sample may be generated by genotyping one or more highly polymorphic SNPs. In some aspects, sequencing, such as base pair and/or paired-end sequencing, may be performed to cover approximately 10%, 15%, 20%, 25%, 30%, 35%, 40%, 45%, 50%, 55%, 60%, 65%, 70%, 75%, 80%, 85%, 90%, 95%, 99%, or greater percentage of target oligonucleotides at, or at more than 20×, 25×, 30×, 35×, 40×, 45×, 50×, or greater than 50× coverage (or any range derivable therein). In certain aspects, mutations, SNPS, INDELS, copy number alterations (somatic and/or germline), or other genetic differences may be identified from the sequencing using at least one bioinformatics tool, including but not limited to, VarScan2, any R package (including CopywriteR) and/or Annovar.

### 2. RNA Sequencing

In some aspects, RNA may be analyzed by sequencing. The RNA may be prepared for sequencing by any method known in the art, such as but not limited to, poly-A selection, cDNA synthesis, stranded or nonstranded library preparation, or a combination thereof. The RNA may be prepared for any type of RNA sequencing technique, including but not limited to, stranded specific RNA sequencing. In some aspects, sequencing may be performed to generate approximately 10M, 15M, 20M, 25M, 30M, 35M, 40M or more reads, including paired reads. In some aspects, the sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer (or any range derivable therein). In some aspects, raw sequencing data may be converted to estimated read counts (RSEM), fragments per kilobase of transcript per million mapped reads (FPKM), and/or reads per kilobase of transcript per million mapped reads (RPKM).

### 3. Example Sequencing Methods

DNA (including bisulfite-converted DNA) and/or RNA (including bisulfite-converted RNA) may be used for amplification of one or more regions of interest followed by sequencing. Accordingly, aspects of the disclosure may include sequencing nucleic acids to detect and/or quantify methylation of nucleic acids biomarkers. In some aspects, the methods of the disclosure include a sequencing method. Sequencing may be excluded from certain methods of the disclosure. Example sequencing methods include, but are not limited to, those described below.

a. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences.

b. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing.

### c. 454 Pyrosequencing™.

A parallelized version of pyrosequencing was developed by 454 Life Sciences™ which has since been acquired by Roche Diagnostics™. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD™ on the other.

d. Illumina™ (Solexa) Sequencing.

Solexa developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

e. SOLiD™ Sequencing.

SOLiD™ technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina™ sequencing.

f. Ion Torrent™ Semiconductor Sequencing.

Ion Torrent™ Systems Inc. developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

g. DNA Nanoballs™ Sequencing.

DNA Nanoballs™ sequencing is a type of high throughput sequencing technology used to determine the entire genomic sequence of an organism. The company Complete Genomics® uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which can make mapping the short reads to a reference genome difficult. This technology has been used for multiple genome sequencing projects.

h. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

i. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### C. Additional Assay Methods

In some aspects, methods involve amplifying and/or sequencing one or more target genomic regions using at least one pair of primers specific to the target genomic regions. In certain aspects, the primers are heptamers. In certain aspects, enzymes are added such as primases or primase/polymerase combination enzyme to the amplification step to synthesize primers.

In some aspects, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, ddPCR (droplet digital PCR), nCounter® (nanoString®), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In some aspects, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In some aspects, quantitative RT-PCR (such as but not limited to TaqMan™, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In some aspects, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In some aspects, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some aspects, the density may be or may be at least 50, 100, 200, 300, 400, 500 or more probes/cm² (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain aspects may involve the use of arrays or data generated from an array. Data may be readily available. Moreover, an array may be prepared in order to generate data that may then be used in correlation studies.

### V. Methods of Use

### A. Identification of DNA Methylation Variants

The field of DNA methylation analysis has expanded recently with the identification of multiple cytosine variants. Traditional DNA methylation involves the transfer of a methyl group to the carbon 5 position of cytosine to produce 5-methylcytosine (5mC). However, research has shown that the Tet family of cytosine oxygenase enzymes are involved in oxidizing 5-methylcytosine into 5-hydroxymethylcytosine (5hmC), 5-formylcytosine (5fC) and 5-carboxylcytosine (5caC).

5-Formylcytosine (5fC) is one of the DNA variants that is produced when Tet enzymes act on 5-hydroxymethylcytosine. Further oxidation of 5-formylcytosine by the Tet enzyme will results in conversion to 5-carboxylcytosine. It is believed that the oxidation of 5-methylcytosine through the various DNA methylation variants represents a mechanism of DNA demethylation, and that this demethylation pathway has a function during development and germ cell programming. 5-Formylcytosine is present in mouse embryonic stem (ES) cells and major mouse organs. This DNA modification also appears in the paternal pronucleus post-fertilization, concomitant with the disappearance of 5-methylcytosine, suggesting its involvement in the DNA demethylation process.

5-Carboxylcytosine (5caC) has been identified as one of the DNA methylation variants that is produced when Tet enzymes oxidize 5-hydroxymethylcytosine and, subsequently 5-formylcytosine. It is believed that the oxidation of 5-methylcytosine through to 5-carboxylcytosine represents a mechanism of DNA demethylation, and that this demethylation pathway has a function during development and germ cell programming. It has been suggested that 5caC is excised from genomic DNA by thymine DNA glycosylase (TDG), which returns the cytosine residue back to its unmodified state. 5-Carboxylcytosine has been identified in mouse embryonic stem (ES) cells. This DNA modification appears in the paternal pronucleus post-fertilization, concomitant with the disappearance of 5-methylcytosine, further lending support that this variant is part of a DNA demethylation pathway.

5-Methylcytosine (5mC) is the DNA modification that results from the transfer of a methyl group from S-adenosyl methionine (also known as AdoMet or SAM) to the carbon 5 position of a cytosine residue. This transfer is catalyzed by DNA methyltransferase enzymes (DNMTs). 5-Methylcytosine is the most common and widely studied form of DNA methylation. It usually occurs within CpG dinucleotide motifs, although non-CpG methylation has been identified in embryonic stem cells.

5-Hydroxymethylcytosine (5hmC) is a DNA methylation modification that occurs as a result of enzymatic oxidation of 5-methylcytosine (5mC) by the Tet family of iron-dependent deoxygenases3. 5-Hydroxymethylcytosine can be found in elevated amounts in certain mammalian tissues, such as mouse Purkinje cells and granule neurons. Alternatively, 5hmC may be produced by the addition of formaldehyde to DNA cytosines by DNMT proteins.

Other methods for distinguishing epigenetic modifications have been provided. It is contemplated that the current methods can be applied and combined with other methods disclosed in the art. Examples of methods disclosed in the art include U.S. Pat. Nos. 8,741,567, 9,611,510, PCT application publication WO201416577, U.S. Pat. No. 11,130,991, and U.S. Patent Application Publication no. 2021/0310062, each of which are hereby incorporated by reference in their entirety. In some aspects, the current methods may include or exclude steps described in the above-referenced patents and patent application publications.

### B. Clinical and Diagnostic Applications

The methods of the disclosure may be useful for evaluating DNA and/or RNA for clinical and/or diagnostic purposes. Certain aspects relate to methods for evaluating DNA. Certain aspects relate to methods for evaluating RNA. Certain aspects relate to a method for evaluating a sample comprising DNA molecules and/or RNA molecules. The evaluation may be the detection or determination of a particular cytosine modification or the differential detection or determination of a particular modification.

The sample may be from a biopsy such as from fine needle aspiration, core needle biopsy, vacuum assisted biopsy, incisional biopsy, excisional biopsy, punch biopsy, shave biopsy or skin biopsy. In certain aspects, the sample is obtained from a biopsy from cancerous tissue by any of the biopsy methods previously mentioned. In certain aspects, the sample may be obtained from any of the tissues provided herein that include but are not limited to gall bladder, skin, heart, lung, breast, pancreas, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In certain aspects, the cyst, tumor or neoplasm is colorectal. In certain aspects of the current methods, any medical professional such as a doctor, nurse or medical technician may obtain a biological sample for testing. Yet further, in certain aspects the biological sample can be obtained without the assistance of a medical professional.

A sample may include but is not limited to, tissue, cells, or biological material from cells or derived from cells of a subject. In some aspects, the sample comprises cell-free DNA. In some aspects, the sample comprises a fertilized egg, a zygote, a blastocyst, or a blastomere. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

In some aspects, the methods of the disclosure can be used in the discovery of novel biomarkers for a disease or condition. In some aspects, the methods of the disclosure can performed on a sample from a patient to provide a prognosis for a certain disease or condition in the patient. In some aspects, the methods of the disclosure can be performed on a sample from a patient to predict the patient's response to a particular therapy. In some aspects, the disease comprises a cancer. For example, the cancer may be pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

In some aspects, the cancer comprises ovarian, prostate, colon, or lung cancer. In some aspects, the method is for determining novel biomarkers for ovarian, prostate, colon, or lung cancer by evaluating cell-free DNA using methods of the disclosure. In some embodiments, the methods of the disclosure may be used on fetal DNA isolated from a pregnant female. In some aspects, the methods of the disclosure may be used for prenatal diagnostics using fetal DNA isolated from a pregnant female. In some aspects, the methods of the disclosure may be used for the evaluation of a fertilized embryo, such as a zygote or a blastocyst for the determination of embryo quality or for the presence or absence of a particular disease marker.

In some aspects, methods disclosed herein are performed on DNA and/or RNA that is at a low input concentration. In some aspects, a low input DNA and/or RNA concentration is at about or below about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11.0, 11.5, 12.0, 12.5, 13.0, 13.5, 14.0, 14.5, or 15 nanograms, or any range derivable therein. In some aspects, a low input DNA and/or RNA concentration is at about 1 to 10 ng, 5 to 10 ng, 10 to 50 ng, or 10 to 100 ng total DNA and/or RNA. In some aspects, a low input concentration of DNA and/or RNA is obtained from about or less than about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, or 500 cells.

### VI. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy, liquid biopsy, or skin biopsy. In certain aspects the sample is obtained from a biopsy from tissue by any of the biopsy methods previously mentioned. In certain aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A biological sample may include but is not limited to, tissue, cells, or biological material from cells or derived from cells of a subject. In some aspects, a biological sample comprises extracellular vesicles such as exosomes. The biological sample may be a heterogeneous or homogeneous population of cells or tissues. A biological sample may be a cell-free sample. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, cerebrospinal fluid collection, urine collection, feces collection, collection of menses, tears, or semen.

The sample may be obtained by methods known in the art. In certain aspects the samples are obtained by biopsy. In certain aspects the sample is obtained by swabbing, endoscopy, scraping, phlebotomy, or any other methods known in the art. In some cases, the sample may be obtained, stored, or transported using components of a kit of the present methods. In some cases, multiple samples may be obtained for diagnosis by the methods described herein. In other cases, multiple samples, such as one or more samples from one tissue and one or more samples from another specimen (for example serum) may be obtained for diagnosis by the methods. In some cases, multiple samples such as one or more samples from one tissue type and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects, the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In some aspects, the sample is a fine needle aspirate of a tissue or a suspected tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VII. Kits

Also disclosed herein are kits, which may be useful for performing the methods of the disclosure. The contents of a kit can include one or more reagents described throughout the disclosure and/or one or more reagents known in the art for performing one or more steps described throughout the disclosure. For example, the kits may include one or more of the following: bisulfite, ammonium bisulfite, ammonium sulfite, ammonium sulfite monohydrate, sodium bisulfite, a bisulfite solution comprising ammonium bisulfite, a bisulfite solution comprising ammonium bisulfite and ammonium sulfite, a 70% ammonium bisulfite solution, a 50% ammonium bisulfite solution, a 50%-70% ammonium bisulfite solution, an APOBEC deaminase enzyme, APOBEC3A, nuclease-free water, one or more primers, polyethylene glycol, magnetic beads, DNA polymerase, taq polymerase, DNA ligase, RNA ligase, a reverse transcriptase, dNTPs, DNA polymerase buffer, RNA polymerase, DTT, redox reagent, Mg²⁺, K⁺, adaptors, DNA adaptors, DNA comprising an RNA promoter, a protease, an alkaline solution, a sodium hydroxide solution, and NTPs. Any one or more of the preceding components may be excluded from a kit in certain aspects of the present disclosure. In some aspects, a kit of the disclosure does not comprise sodium bisulfite or added sodium bisulfite. In some aspects, a kit of the disclosure does not comprise ammonium sulfite or added ammonium sulfite.

In certain aspects, a kit of the disclosure comprises a solution comprising ammonium bisulfite. In some aspects, the solution comprises between 50% and 70% ammonium bisulfite by weight, including any range or value derivable therein. In some aspects, a kit of the disclosure comprises a solution comprising at least, at most, or about 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7%, 55.8%, 55.9%, 56%, 56.1%, 56.2%, 56.3%, 56.4%, 56.5%, 56.6%, 56.7%, 56.8%, 56.9%, 57%, 57.1%, 57.2%, 57.3%, 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5%, 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, or 70% ammonium bisulfite by weight, or any range or value derivable therein. In some aspects, the solution comprises at least, at most, or about 66%, 66.01%, 66.02%, 66.03%, 66.04%, 66.05%, 66.06%, 66.07%, 66.08%, 66.09%, 66.1%, 66.11%, 66.12%, 66.13%, 66.14%, 66.15%, 66.16%, 66.17%, 66.18%, 66.19%, 66.2%, 66.21%, 66.22%, 66.23%, 66.24%, 66.25%, 66.26%, 66.27%, 66.28%, 66.29%, 66.3%, 66.31%, 66.32%, 66.33%, 66.34%, 66.35%, 66.36%, 66.37%, 66.38%, 66.39%, 66.4%, 66.41%, 66.42%, 66.43%, 66.44%, 66.45%, 66.46%, 66.47%, 66.48%, 66.49%, 66.5%, 66.51%, 66.52%, 66.53%, 66.54%, 66.55%, 66.56%, 66.57%, 66.58%, 66.59%, 66.6%, 66.61%, 66.62%, 66.63%, 66.64%, 66.65%, 66.66%, 66.67%, 66.68%, 66.69%, 66.7%, 66.71%, 66.72%, 66.73%, 66.74%, 66.75%, 66.76%, 66.77%, 66.78%, 66.79%, 66.8%, 66.81%, 66.82%, 66.83%, 66.84%, 66.85%, 66.86%, 66.87%, 66.88%, 66.89%, 66.9%, 66.91%, 66.92%, 66.93%, 66.94%, 66.95%, 66.96%, 66.97%, 66.98%, 66.99%, or 67% ammonium bisulfite by weight, or any range or value derivable therein. In some aspects, the solution comprises about 66.67% ammonium bisulfite by weight.

In some aspects, the solution comprises ammonium sulfite. In some aspects, the solution comprises at least, at most, or about 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, or 15% ammonium sulfite by weight, or any range or value derivable therein. In some aspects, the solution comprises ammonium sulfite at a concentration of, or of less than 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, or less. In some aspects, the solution comprises less than 1%, 0.1%, 0.01%, 0.001%, or 0.0001% ammonium bisulfite by weight, or less. In some aspects, the solution comprises less than 1%, 0.1%, 0.01%, 0.001%, or 0.0001% ammonium sulfite by weight, or less. In certain aspects the solution does not comprise ammonium sulfite or added ammonium sulfite.

In some aspects, the solution does not comprise ammonium sulfite or added ammonium sulfite. In some aspects, the solution comprises ammonium sulfite at a concentration of, or of less than 1 M, 0.9 M, 0.8 M, 0.7 M, 0.6 M, 0.5 M, 0.4 M, 0.3 M, 0.2 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or less.

In some aspects, the solution is at a bisulfite concentration between 6.5 M and 10 M, including any range or value derivable therein. In some aspects, the solution is at a bisulfite concentration of at least, at most, or about 6.5 M, 6.6 M, 6.7 M, 6.8 M, 6.9 M, 7 M, 7.1 M, 7.2 M, 7.3 M, 7.4 M, 7.5 M, 7.6 M, 7.7 M, 7.8 M, 7.9 M, 8 M, 8.1 M, 8.2 M, 8.3 M, 8.4 M, 8.5 M, 8.6 M, 8.7 M, 8.8 M, 8.9 M, 9 M, 9.1 M, 9.2 M, 9.3 M, 9.4 M, 9.5 M, 9.6 M, 9.7 M, 9.8 M, 9.9 M, or 10 M, or any range or value derivable therein. In some aspects, the solution is at a bisulfite concentration of about 7.0 M. In some aspects, the solution is at a bisulfite concentration of 7.0 M. In some aspects, the solution is at a bisulfite concentration of about 9.5 M. In some aspects, the solution is at a bisulfite concentration of about 9.5 M. In some aspects, the solution has a pH between 4.8 and 5.4, including any range or value derivable therein. In some aspects, the solution has a pH of at least, at most, or about 4.8, 4.9, 5, 5.1, 5.2, 5.3, or 5.4. In some aspects, the solution has a pH of about 5.1.

In some aspects, the solution does not comprise sodium bisulfite or added sodium bisulfite. In some aspects, the solution comprises sodium at a concentration of less than 1 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or less. In some aspects, the solution does not comprise sodium.

In some aspects, the solution does not comprise sodium bisulfite or added sodium bisulfite. In some aspects, the solution comprises sodium bisulfite at a concentration of, or of less than 1 M, 0.9 M, 0.8 M, 0.7 M, 0.6 M, 0.5 M, 0.4 M, 0.3 M, 0.2 M, 0.1 M, 0.01 M, 1×10⁻³ M, 1×10⁻⁴ M, 1×10⁻⁵ M, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, 1×10⁻¹⁵ M, 1×10⁻¹⁶ M, 1×10⁻¹⁷ M, 1×10⁻¹⁸ M, 1×10⁻¹⁹ M, 1×10⁻²⁰ M, or less. In some aspects, the solution comprises less than 1%, 0.1%, 0.01%, 0.001%, or 0.0001% sodium bisulfite by weight, or less.

In certain aspects, a kit of the disclosure comprises instructions for processing a nucleic acid sample, such as a DNA sample or an RNA sample. Instructions may comprise instructions for using one or more components of the kit in a method disclosed herein. For example, instructions may include one or more of instructions for incubating a nucleic acid sample with a bisulfite solution, instructions for mixing a bisulfite solution and a nucleic acid sample, instructions for bisulfite treatment of a nucleic acid, instructions for isolating nucleic acid from a sample, instructions for nucleic acid amplification, and instructions for preparing a sample for sequencing. Instructions for incubating a nucleic acid sample with a bisulfite solution may comprise instructions for incubating the sample and the solution for, or for at most 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, or 1 minute, or less, or any range or value derivable therein. Instructions for incubating a nucleic acid sample with a bisulfite solution may comprise instructions for incubating the sample and the solution at a temperature of, or of at least 80° C., 80.1° C., 80.2° C., 80.3° C., 80.4° C., 80.5° C., 80.6° C., 80.7° C., 80.8° C., 80.9° C., 81° C., 81.1° C., 81.2° C., 81.3° C., 81.4° C., 81.5° C., 81.6° C., 81.7° C., 81.8° C., 81.9° C., 82° C., 82.1° C., 82.2° C., 82.3° C., 82.4° C., 82.5° C., 82.6° C., 82.7° C., 82.8° C., 82.9° C., 83° C., 83.1° C., 83.2° C., 83.3° C., 83.4° C., 83.5° C., 83.6° C., 83.7° C., 83.8° C., 83.9° C., 84° C., 84.1° C., 84.2° C., 84.3° C., 84.4° C., 84.5° C., 84.6° C., 84.7° C., 84.8° C., 84.9° C., 85° C., 85.1° C., 85.2° C., 85.3° C., 85.4° C., 85.5° C., 85.6° C., 85.7° C., 85.8° C., 85.9° C., 86° C., 86.1° C., 86.2° C., 86.3° C., 86.4° C., 86.5° C., 86.6° C., 86.7° C., 86.8° C., 86.9° C., 87° C., 87.1° C., 87.2° C., 87.3° C., 87.4° C., 87.5° C., 87.6° C., 87.7° C., 87.8° C., 87.9° C., 88° C., 88.1° C., 88.2° C., 88.3° C., 88.4° C., 88.5° C., 88.6° C., 88.7° C., 88.8° C., 88.9° C., 89° C., 89.1° C., 89.2° C., 89.3° C., 89.4° C., 89.5° C., 89.6° C., 89.7° C., 89.8° C., 89.9° C., 90° C., 90.1° C., 90.2° C., 90.3° C., 90.4° C., 90.5° C., 90.6° C., 90.7° C., 90.8° C., 90.9° C., 91° C., 91.1° C., 91.2° C., 91.3° C., 91.4° C., 91.5° C., 91.6° C., 91.7° C., 91.8° C., 91.9° C., 92° C., 92.1° C., 92.2° C., 92.3° C., 92.4° C., 92.5° C., 92.6° C., 92.7° C., 92.8° C., 92.9° C., 93° C., 93.1° C., 93.2° C., 93.3° C., 93.4° C., 93.5° C., 93.6° C., 93.7° C., 93.8° C., 93.9° C., 94° C., 94.1° C., 94.2° C., 94.3° C., 94.4° C., 94.5° C., 94.6° C., 94.7° C., 94.8° C., 94.9° C., 95° C., 95.1° C., 95.2° C., 95.3° C., 95.4° C., 95.5° C., 95.6° C., 95.7° C., 95.8° C., 95.9° C., 96° C., 96.1° C., 96.2° C., 96.3° C., 96.4° C., 96.5° C., 96.6° C., 96.7° C., 96.8° C., 96.9° C., 97° C., 97.1° C., 97.2° C., 97.3° C., 97.4° C., 97.5° C., 97.6° C., 97.7° C., 97.8° C., 97.9° C., 98° C., 98.1° C., 98.2° C., 98.3° C., 98.4° C., 98.5° C., 98.6° C., 98.7° C., 98.8° C., 98.9° C., 99° C., 99.1° C., 99.2° C., 99.3° C., 99.4° C., 99.5° C., 99.6° C., 99.7° C., 99.8° C., 99.9° C., or more, or any range or value derivable therein. In some aspects, the instructions comprise instructions for incubating the sample at about 98° C. In some aspects, the instructions comprise instructions for incubating the sample at 98° C.

One or more reagent is preferably supplied in a solid form or liquid buffer that is suitable for inventory storage, and later for addition into the reaction medium when the method of using the reagent is performed. Suitable packaging is provided. The kit may provide additional components that are useful in the procedure. These additional components may include buffers, capture reagents, developing reagents, labels, reacting surfaces, means for detection, control samples, instructions, and interpretive information.

Any components of a kit described herein may be used in a method disclosed herein. Further, components described in the context of a disclosed method may be provided in a kit of the present disclosure.

### VIII. Aspects

The following non-limiting aspects are included to demonstrate certain features of the inventions disclosed herein.

Aspect 1. A method for DNA processing, the method comprising: (a) incubating a solution comprising a DNA molecule and ammonium bisulfite at a temperature of at least 95° C. for at most 12 minutes, wherein the solution does not comprise added sodium bisulfite; and (b) subjecting the DNA molecule to alkaline conditions.

Aspect 2. The method of aspect 1, wherein the solution does not comprise added ammonium sulfite.

Aspect 3. The method of aspect 1 or 2, wherein the solution does not comprise ammonium sulfite at levels greater than about 1/10^{th} the levels of ammonium bisulfite.

Aspect 4. The method of any of aspects 1-3, wherein the solution does not comprise sodium bisulfite at levels greater than about 1/10^{th} the levels of ammonium bisulfite.

Aspect 5. The method of any of aspects 1-4, wherein the solution is at a bisulfite concentration between 6.5 M and 10 M.

Aspect 6. The method of any of aspects 1-5, wherein the solution is at a bisulfite concentration between 8 M and 10 M.

Aspect 7. The method of any of aspects 1-6, wherein the solution is at a bisulfite concentration between 9 M and 10 M.

Aspect 8. The method of any of aspects 1-7, wherein the solution is at a bisulfite concentration of about 9.5 M.

Aspect 9. The method of any of aspects 1-8, wherein the solution comprises between 50% and 70% ammonium bisulfite by weight.

Aspect 10. The method of any of aspects 1-9, wherein the solution comprises between 60% and 70% ammonium bisulfite by weight.

Aspect 11. The method of any of aspects 1-10, wherein the solution comprises between 65% and 68% ammonium bisulfite by weight.

Aspect 12. The method of any of aspects 1-11, wherein the solution comprises about 66.7% ammonium bisulfite by weight.

Aspect 13. The method of any of aspects 1-12, wherein the solution has a pH between 4.8-5.4.

Aspect 14. The method of any of aspects 1-13, wherein the solution has a pH of about 5.1.

Aspect 15. The method of any of aspects 1-14, wherein (a) comprises incubating the solution at a temperature of about 98° C.

Aspect 16. The method of any of aspects 1-15, wherein (a) comprises incubating the solution for at most 10 minutes.

Aspect 17. The method of any of aspects 1-16, wherein (a) comprises incubating the solution for at most 8 minutes.

Aspect 18. The method of any of aspects 1-17, wherein the DNA molecule comprises 4mC, and greater than 50% of the 4mC is deaminated after the incubation.

Aspect 19. The method of any of aspects 1-18, wherein greater than 75% of the 4mC is deaminated after the incubation.

Aspect 20. The method of any of aspects 1-19, wherein substantially all of the 4mC is deaminated after the incubation.

Aspect 21. A method for DNA processing, the method comprising: (a) generating a solution comprising a DNA molecule and ammonium bisulfite, wherein the solution does not comprise added sodium bisulfite; (b) incubating the solution at a temperature of at least 95° C.; and (c) removing the DNA molecule from the solution at most 12 minutes after (a).

Aspect 22. The method of aspect 21, wherein the solution does not comprise added ammonium sulfite.

Aspect 23. The method of aspect 21 or 22, wherein the solution does not comprise ammonium sulfite at levels greater than about 1/10^{th} the levels of ammonium bisulfite.

Aspect 24. The method of any of aspects 21-23, wherein the solution does not comprise sodium bisulfite at levels greater than about 1/10^{th} the levels of ammonium bisulfite.

Aspect 25. The method of any of aspects 21-24, wherein the solution is at a bisulfite concentration between 6.5 M and 10 M.

Aspect 26. The method of any of aspects 21-25, wherein the solution is at a bisulfite concentration between 8 M and 10 M.

Aspect 27. The method of any of aspects 21-26, wherein the solution is at a bisulfite concentration between 9 M and 10 M.

Aspect 28. The method of any of aspects 21-27, wherein the solution is at a bisulfite concentration of about 9.5 M.

Aspect 29. The method of any of aspects 21-28, wherein the solution comprises between 50% and 70% ammonium bisulfite by weight.

Aspect 30. The method of any of aspects 21-29, wherein the solution comprises between 60% and 70% ammonium bisulfite by weight.

Aspect 31. The method of any of aspects 21-30, wherein the solution comprises between 65% and 68% ammonium bisulfite by weight.

Aspect 32. The method of any of aspects 21-31, wherein the solution comprises about 66.7% ammonium bisulfite by weight.

Aspect 33. The method of any of aspects 21-32, wherein the solution has a pH between 4.8-5.4.

Aspect 34. The method of any of aspects 21-33, wherein the solution has a pH of about 5.1.

Aspect 35. The method of any of aspects 21-34, wherein (b) comprises incubating the solution at a temperature of about 98° C.

Aspect 36. The method of any of aspects 21-35, wherein (c) comprises removing the DNA molecule from the solution at most 10 minutes after (a).

Aspect 37. The method of any of aspects 21-36, wherein (c) comprises removing the DNA molecule from the solution at most 8 minutes after (a).

Aspect 38. The method of any of aspects 21-37, wherein (a) comprises mixing a 70% ammonium bisulfite solution and a 50% bisulfite solution.

Aspect 39. The method of any of aspects 21-38, wherein the DNA molecule comprises 4mC, and greater than 50% of the 4mC is deaminated after the incubation.

Aspect 40. The method of any of aspects 21-39, wherein greater than 75% of the 4mC is deaminated after the incubation.

Aspect 41. The method of any of aspects 21-40, wherein substantially all of the 4mC is deaminated after the incubation.

Aspect 42. A method for processing a nucleic acid sample, the method comprising incubating a solution comprising DNA molecules and ammonium bisulfite at a temperature of at least 95° C. for at most 12 minutes, wherein the solution does not comprise added sodium bisulfite, wherein the DNA molecules each comprise one or more cytosine residues, wherein, after incubating the solution, greater than 99% of the DNA molecules comprise no cytosine residue.

Aspect 43. The method of aspect 42, wherein the solution does not comprise sodium bisulfite at levels greater than about 1/10^{th} the levels of ammonium bisulfite.

Aspect 44. The method of aspect 42 or 43, further comprising subjecting the plurality of DNA molecules to alkaline conditions.

Aspect 45. The method of any of aspects 42-44, wherein the solution comprises between 50% and 70% ammonium bisulfite by weight.

Aspect 46. The method of any of aspects 42-45, wherein the solution comprises between 60% and 70% ammonium bisulfite by weight.

Aspect 47. The method of any of aspects 42-46, wherein the solution comprises between 65% and 68% ammonium bisulfite by weight.

Aspect 48. The method of any of aspects 42-47, wherein the solution comprises about 66.7% ammonium bisulfite by weight.

Aspect 49. The method of any of aspects 42-48, wherein the solution does not comprise added ammonium sulfite.

Aspect 50. The method of any of aspects 42-49, wherein the solution does not comprise ammonium sulfite at levels greater than about 1/10^{th} the levels of ammonium bisulfite.

Aspect 51. The method of any of aspects 42-50, wherein the solution is at a bisulfite concentration between 6.5 M and 10 M.

Aspect 52. The method of any of aspects 42-51, wherein the solution is at a bisulfite concentration between 8 M and 10 M.

Aspect 53. The method of any of aspects 42-52, wherein the solution is at a bisulfite concentration between 9 M and 10 M.

Aspect 54. The method of any of aspects 42-53, wherein the solution is at a bisulfite concentration of about 9.5 M.

Aspect 55. The method of any of aspects 42-54, wherein the solution has a pH between 4.8-5.4.

Aspect 56. The method of any of aspects 42-55, wherein the DNA molecule comprises 4mC, and greater than 50% of the 4mC is deaminated after the incubation.

Aspect 57. The method of any of aspects 42-56, wherein greater than 75% of the 4mC is deaminated after the incubation.

Aspect 58. The method of any of aspects 42-57, wherein substantially all of the 4mC is deaminated after the incubation.

Aspect 59. A DNA processing kit comprising: (a) a solution comprising ammonium bisulfite having a bisulfite concentration between 6.5 M and 10 M, wherein the solution does not comprise sodium bisulfite; and (b) instructions for processing a DNA sample.

Aspect 60. The kit of aspect 59, wherein the solution does not comprise sodium bisulfite at levels greater than about 1/10^{th} the levels of ammonium bisulfite

Aspect 61. The kit of aspect 59 or 60, wherein the solution is at a bisulfite concentration between 8 M and 10 M.

Aspect 62. The kit of any of aspects 59-61, wherein the solution is at a bisulfite concentration between 9 M and 10 M.

Aspect 63. The kit of any of aspects 59-62, wherein the solution is at a bisulfite concentration of about 9.5 M.

Aspect 64. The kit of any of aspects 59-63, wherein the solution comprises between 50% and 70% ammonium bisulfite by weight.

Aspect 65. The kit of any of aspects 59-64, wherein the solution comprises between 60% and 70% ammonium bisulfite by weight.

Aspect 66. The kit of any of aspects 59-65, wherein the solution comprises between 65% and 68% ammonium bisulfite by weight.

Aspect 67. The kit of any of aspects 59-66, wherein the solution comprises about 66.7% ammonium bisulfite by weight.

Aspect 68. The kit of any of aspects 59-67, wherein the solution has a pH between 4.8-5.4.

Aspect 69. The kit of any of aspects 59-68, wherein the solution has a pH of about 5.1.

Aspect 70. The kit of any of aspects 59-69, wherein the instructions comprise instructions for incubating the DNA sample with the solution at a temperature of at least 95° C. for at most 12 minutes.

Aspect 71. The kit of any of aspects 59-70, wherein the instructions comprise instructions for incubating the DNA sample with the solution at a temperature of about 98° C.

Aspect 72. The kit of any of aspects 59-71, wherein the instructions comprise instructions for incubating the DNA sample with the solution for at most 10 minutes.

Aspect 73. The kit of any of aspects 59-72, wherein the instructions comprise instructions for incubating the DNA sample with the solution for at most 8 minutes.

Aspect 74. The kit of any of aspects 59-73, wherein the solution does not comprise ammonium sulfite.

Aspect 75. The kit of any of aspects 59-74, wherein the solution does not comprise ammonium sulfite at levels greater than about 1/10^{th} the levels of ammonium bisulfite.

Aspect 76. The kit of any of aspects 59-75, further comprising an alkaline solution.

Aspect 77. The kit of any of aspects 59-76, further comprising one or more buffer solutions.

Aspect 78. A method for RNA processing, the method comprising: (a) incubating a solution comprising an RNA molecule, ammonium sulfite, and ammonium bisulfite at a temperature of at least 95° C. for at most 12 minutes, wherein the solution does not comprise added sodium bisulfite; and (a) subjecting the RNA molecule to alkaline conditions.

Aspect 79. The method of aspect 78, wherein the solution does not comprise sodium bisulfite at levels greater than about 1/10^{th} the levels of ammonium sulfite.

Aspect 80. The method of aspect 78 or 79, wherein the solution does not comprise sodium bisulfite at levels greater than about 1/10^{th} the levels of ammonium bisulfite.

Aspect 81. The method of any of aspects 78-80, wherein the solution is at a bisulfite concentration between 6.5 M and 10 M.

Aspect 82. The method of any of aspects 78-81, wherein the solution is at a bisulfite concentration between 6.5 M and 7.5 M.

Aspect 83. The method of any of aspects 78-82, wherein the solution is at a bisulfite concentration of about 7.0 M.

Aspect 84. The method of any of aspects 78-83, wherein the solution has a pH between 4.8-5.4.

Aspect 85. The method of any of aspects 78-84, wherein the solution comprises between 5% and 15% ammonium sulfite by weight.

Aspect 86. The method of any of aspects 78-85, wherein the solution comprises between 8% and 12% ammonium sulfite by weight.

Aspect 87. The method of any of aspects 78-86, wherein the solution comprises about 10% ammonium sulfite by weight.

Aspect 88. The method of any of aspects 78-87, wherein (a) comprises incubating the solution at a temperature of about 98° C.

Aspect 89. The method of any of aspects 78-88, wherein (a) comprises incubating the solution for at most 10 minutes.

Aspect 90. The method of any of aspects 78-88, wherein (a) comprises incubating the solution for at most 8 minutes.

Aspect 91. A method for RNA processing, the method comprising: (a) generating a solution comprising an RNA molecule, ammonium sulfite, and ammonium bisulfite, wherein the solution does not comprise added sodium bisulfite; (b) incubating the solution at a temperature of at least 95° C.; and (c) removing the RNA molecule from the solution at most 12 minutes after (a).

Aspect 92. The method of aspect 91, wherein the solution does not comprise sodium bisulfite at levels greater than about 1/10^{th} the levels of ammonium sulfite.

Aspect 93. The method of aspect 91 or 92, wherein the solution does not comprise sodium bisulfite at levels greater than about 1/10^{th} the levels of ammonium bisulfite.

Aspect 94. The method of any of aspects 91-93, wherein the solution has a bisulfite concentration between 6.5 M-10 M.

Aspect 95. The method of any of aspects 91-94, wherein the solution has a bisulfite concentration between 6.5 M and 7.5 M.

Aspect 96. The method of any of aspects 91-95, wherein the solution has a bisulfite concentration of about 7.0 M.

Aspect 97. The method of any of aspects 91-96, wherein the solution has a pH between 4.8-5.4.

Aspect 98. The method of any of aspects 91-97, wherein the solution has a pH of about 5.1.

Aspect 99. The method of any of aspects 91-98, wherein the solution comprises between 5% and 15% ammonium sulfite by weight.

Aspect 100. The method of any of aspects 91-99, wherein the solution comprises between 8% and 12% ammonium sulfite by weight.

Aspect 101. The method of any of aspects 91-100, wherein the solution comprises about 10% ammonium sulfite by weight.

Aspect 102. The method of any of aspects 91-101, wherein (b) comprises incubating the solution at a temperature of about 98° C.

Aspect 103. The method of any of aspects 91-102, wherein (c) comprises removing the RNA molecule from the solution at most 10 minutes after (a).

Aspect 104. The method of any of aspects 91-103, wherein (c) comprises removing the RNA molecule from the solution at most 8 minutes after (a).

Aspect 105. A method for processing a nucleic acid sample, the method comprising incubating a solution comprising RNA molecules, ammonium sulfite, and ammonium bisulfite at a temperature of at least 95° C. for at most 12 minutes, wherein the solution does not comprise added sodium bisulfite, wherein the RNA molecules each comprise one or more cytosine residues, wherein, after incubating the solution, greater than 99% of the RNA molecules comprise no cytosine residue.

Aspect 106. The method of aspect 105, wherein the solution does not comprise sodium bisulfite at levels greater than about 1/10^{th} the levels of ammonium sulfite.

Aspect 107. The method of aspect 105 or 106, wherein the solution does not comprise sodium bisulfite at levels greater than about 1/10^{th} the levels of ammonium bisulfite.

Aspect 108. The method of any of aspects 105-107, wherein the solution has a pH between 4.8-5.4.

Aspect 109. The method of any of aspects 105-108, wherein the solution has a pH of about 5.1.

Aspect 110. The method of any of aspects 105-109, wherein the solution comprises between 5% and 15% ammonium sulfite by weight.

Aspect 111. The method of any of aspects 105-110, wherein the solution comprises between 8% and 12% ammonium sulfite by weight.

Aspect 112. The method of any of aspects 105-111, wherein the solution comprises about 10% ammonium sulfite by weight.

Aspect 113. The method of any of aspects 105-112, wherein (a) comprises incubating the solution at a temperature of about 98° C.

Aspect 114. The method of any of aspects 105-113, wherein (a) comprises incubating the solution for at most 10 minutes.

Aspect 115. The method of any of aspects 105-114, wherein (a) comprises incubating the solution for at most 8 minutes.

Aspect 116. The method of any of aspects 105-115, wherein the solution has a bisulfite concentration between 6.5 M-10 M.

Aspect 117. The method of any of aspects 105-116, wherein the solution has a bisulfite concentration between 6.5 M and 7.5 M

Aspect 118. The method of any of aspects 105-117, wherein the solution has a bisulfite concentration of about 7.0 M.

Aspect 119. The method of any of aspects 105-118, further comprising subjecting the plurality of RNA molecules to alkaline conditions.

Aspect 120. An RNA processing kit comprising: (a) a solution comprising ammonium sulfite and ammonium bisulfite at a bisulfite concentration between 6.5 M-8 M, wherein the solution does not comprise added sodium bisulfite; and (b) instructions for processing an RNA sample.

Aspect 121. The kit of aspect 120, wherein the solution does not comprise sodium bisulfite at levels greater than about 1/10^{th} the levels of ammonium sulfite.

Aspect 122. The kit of aspect 120 or 121, wherein the solution does not comprise sodium bisulfite at levels greater than about 1/10^{th} the levels of ammonium bisulfite.

Aspect 123. The kit of any of aspects 120-122, wherein the solution is at a bisulfite concentration of about 7.0 M.

Aspect 124. The kit of any of aspects 120-123, wherein the solution has a pH between 4.8-5.4.

Aspect 125. The kit of any of aspects 120-124, wherein the solution has a pH of about 5.1.

Aspect 126. The kit of any of aspects 120-125, wherein the solution comprises between 5% and 15% ammonium sulfite by weight.

Aspect 127. The kit of any of aspects 120-126, wherein the solution comprises between 8% and 12% ammonium sulfite by weight.

Aspect 128. The kit of any of aspects 120-127, wherein the solution comprises about 10% ammonium sulfite by weight.

Aspect 129. The kit of any of aspects 120-128, wherein the instructions comprise instructions for incubating the RNA sample with the solution at a temperature of at least 95° C. for at most 12 minutes.

Aspect 130. The kit of any of aspects 120-129, wherein the instructions comprise instructions for incubating the RNA sample with the solution at a temperature of about 98° C.

Aspect 131. The kit of any of aspects 120-130, wherein the instructions comprise instructions for incubating the RNA sample with the solution for at most 10 minutes.

Aspect 132. A method for 5-hydroxymethylcytosine analysis, the method comprising: (a) incubating a first solution comprising a first DNA molecule and ammonium bisulfite at a temperature of at least 95° C. for at most 12 minutes; (b) incubating a second solution comprising a second DNA molecule and ammonium bisulfite at a temperature of at least 95° C. for at most 12 minutes; (c) subjecting the first DNA molecule to alkaline conditions; (d) subjecting the second DNA molecule to alkaline conditions; (e) treating the second DNA molecule with an APOBEC deaminase enzyme; and (f) sequencing the first DNA molecule and the second DNA molecule.

Aspect 133. The method of aspect 132, wherein the first solution does not comprise added sodium bisulfite.

Aspect 134. The method of aspect 132 or 133, wherein the first solution does not comprise sodium bisulfite at levels greater than about 1/10^{th} the levels of ammonium bisulfite.

Aspect 135. The method of any of aspects 132-134, wherein the second solution does not comprise added sodium bisulfite.

Aspect 136. The method of any of aspects 132-135, wherein the second solution does not comprise sodium bisulfite at levels greater than about 1/10^{th} the levels of ammonium bisulfite.

Aspect 137. The method of any of aspects 132-136, wherein the first solution and the second solution are the same solution.

Aspect 138. The method of any of aspects 132-136, wherein the first solution and the second solution are different solutions.

Aspect 139. The method of any of aspects 132-138, wherein (a) and (b) are performed simultaneously.

Aspect 140. The method of any of aspects 132-139, wherein (c) and (d) are performed simultaneously.

Aspect 141. The method of any of aspects 132-140, wherein the first DNA molecule and the second DNA molecule have the same nucleotide sequence.

Aspect 142. The method of any of aspects 132-141, wherein the APOBEC deaminase enzyme is APOBEC3A.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Bisulfite Sequencing for m⁵C Detection and Analysis in RNA

RNA m⁵C modification and its regulators have been shown to impact diverse cellular functions and play important roles in the pathogenesis of bladder cancer¹, hepato-cellular carcinoma (HCC)², glioblastoma multiforme (GBM)³ and leukemia⁴, suggesting regulatory roles of RNA m⁵C modification. Various methods such as m⁵C-RIP-seq⁵, 5-azacytidine-mediated RNA immunoprecipitation (Aza-IP)⁶ and miCLIP⁷ have been reported for transcriptome-wide m⁵C mapping in RNA, but they all include an antibody enrichment step. BS sequencing remains the gold standard for 5mC sequencing and has been increasingly applied to study RNA m⁵C in recent years,^{1,8-10}. Several commercial RNA BS conversion kits are available, including the EZ RNA Methylation™ Kit from Zymo Research and Methylamp™ RNA BS Conversion Kit from Epigentek. Besides providing a transcriptome-wide view of m⁵C deposition at single-nucleotide resolution, RNA BS sequencing is inexpensive and easy to work with. On the other hand, although BS-seq was effective in detecting m⁵C in abundant RNAs such as tRNA and rRNA^{11,12}, a great discrepancy was observed in different studies in low abundant RNA such as mRNA, from some studies detecting m⁵C sites in 8000 different RNAs¹³, to other studies finding only a few methylated mRNAs¹⁴. More recent studies have reported only few hundred m⁵C sites in human and mouse transcriptomes using an improved bisulfite sequencing method and a more stringent computational approachi¹⁵. These controversial findings have raised the need to develop more robust methods for identifying real m⁵C sites in mRNA¹⁵.

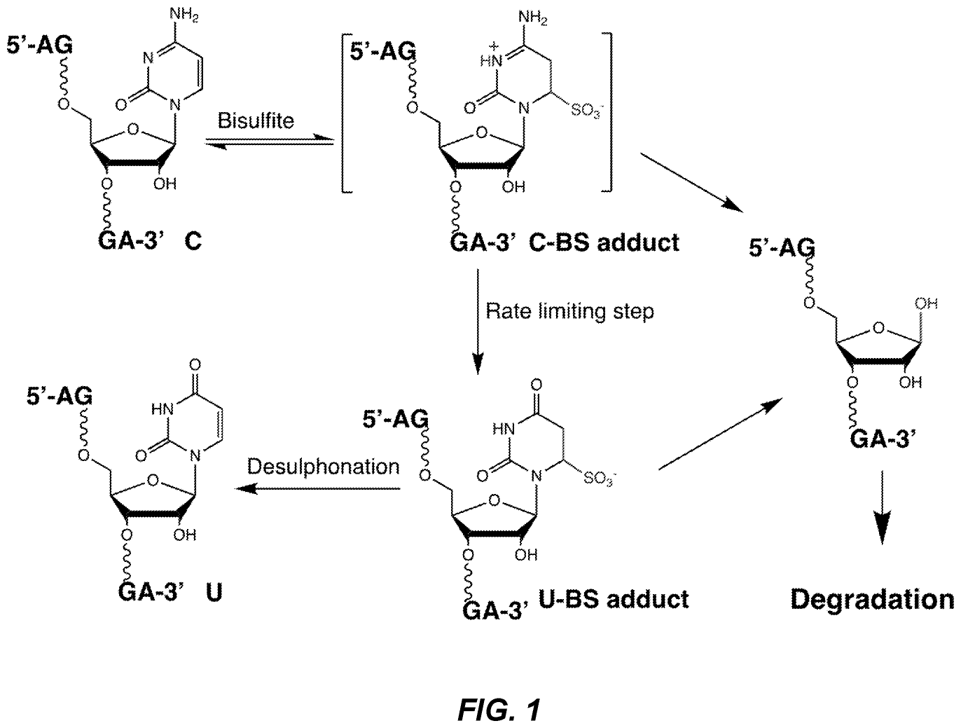

In a comprehensive effort to develop quantitative methods to sequence various RNA modifications, the inventors focused on: (i) reducing RNA degradation by improving efficiency so that the reaction can be completed in a very short period of time; and (ii) using high reaction temperature to denature RNA to achieve complete C-to-U conversion. In the mechanism of bisulfite (BS) sequencing (FIG. 1), the reaction of cytosine with BS is fast and reversible, while the deamination to convert the C-BS adduct to the U-BS adduct is rate-limiting¹⁶. Bisulfite is involved in both steps^{16,17}, therefore, the BS conversion rate can be dramatically accelerated when increased BS concentration is used¹⁷. Application of this strategy to RNA m⁵C sequencing has not been reported.

C-BS and U-BS adducts are the major species that generate abasic sites leading to further RNA cleavage and degradation¹⁹. It was reasoned that a fast conversion of C-to-U would reduce the time that both C-BS and U-BS would exist in the reaction, and therefore reduce RNA damage. In addition, it was further reasoned that higher temperature would not only accelerate deamination reaction but, more importantly, help to denature secondary structures in RNA so that a complete bisulfite conversion can be accomplished within much shorter time. Although high BS concentration and high reaction temperature might hypothetically cause more RNA damage, it was hypothesized that a much shorter reaction time could decrease RNA damage, and thus ultimately reduce RNA degradation. It was also important that high concentrations of BS and high temperatures did not cause undesired deamination of m⁵C.

Current bisulfite treatments are run at 3-5 M bisulfite concentration due to the limited solubility of sodium salts of bisulfite in water. In DNA 5mC BS-sequencing, Shiraishi et al proposed that ammonium bisulfite has higher solubility in water and their proposed high concentration of bisulfite reagent was reported to be more efficient than bisulfite reagents with lower concentration prepared from sodium saltis,²⁰. Shiraishi et al proposed using ammonium bisulfite mixed with sodium bisulfite to obtain a 10 M bisulfite reagent (2.08 g NaHSO₃, 0.67 g ammonium sulfite monohydrate in 5.0 mL 50% ammonium bisulfite) for DNA 5mC sequencing^{18,20}. In attempting to reproduce these conditions, it was found that the mixture prepared according to this recipe needed to be heated in order to dissolve the solid and that the bisulfite salts precipitated easily when the solution was cooled down to room temperature. In addition, it was found that solution was very sticky and therefore difficult to handle and not a consistent recipe.

The inventors next generated bisulfite recipes consisting of only ammonium bisulfite and ammonium sulfite. A series of BS conditions were screened such as BS salts, concentrations, pH, temperature, and reaction time.

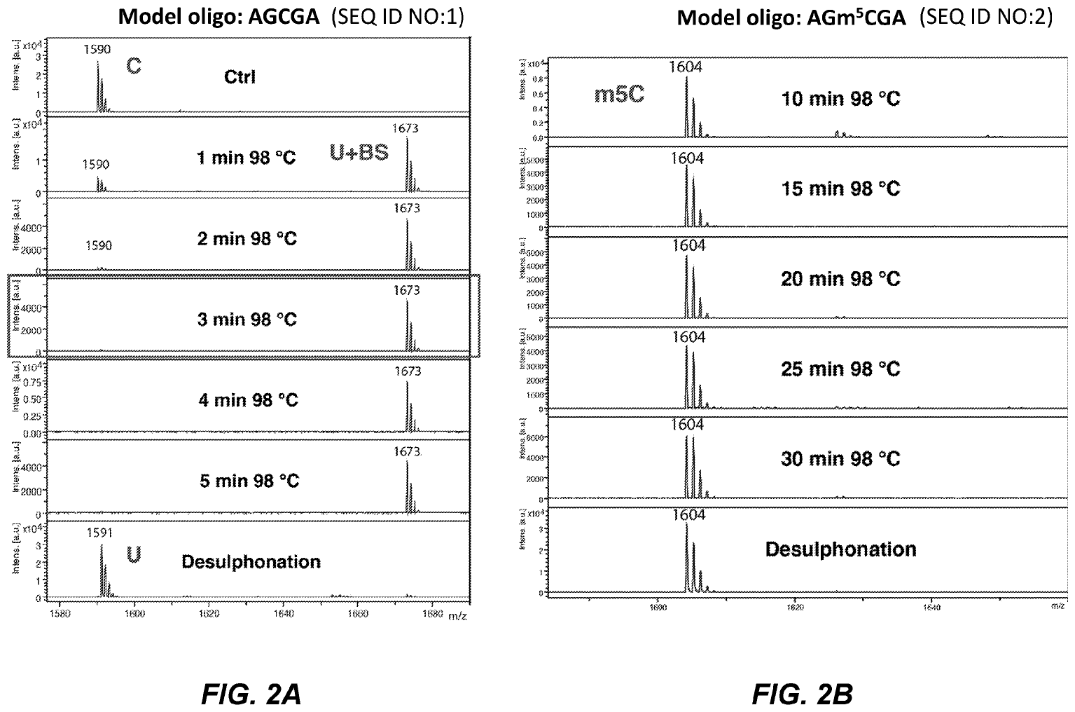

The mixture comprising 50% ammonium bisulfite (1 mL) and ammonium sulfite (100 mg) as a clear solution (termed “R-1G”; bisulfite concentration ˜7.0 M, pH ˜5.1) was mixed (9 μL) with a 5mer RNA oligo AGCGA (SEQ ID NO: 1) (100 ng) in water (1 μL) and incubated at 98° C. MALDI TOF mass spectrometry (MS) was used to monitor the reaction. Since neither A nor G reacts with BS, the mass change before and after BS treatment should only reflect the reaction of cytosine residue with BS. As shown in FIG. 2A, the majority of starting material was consumed within 1 minute, giving an intermediate with MS of +83, suggesting that cytosine was converted to the corresponding U-BS adduct directly. Cytosine was almost totally consumed within 2 min and completely converted to U-BS adduct within 3 min.

Interestingly, no C-BS adduct (with MS of +82 compared with control) was observed at any time point in the reaction, suggesting that using the new BS recipe, the deamination of C-BS to form U-BS adduct was dramatically accelerated so that it was no longer the rate-limiting step. This observation likely explains why the new BS conditions can dramatically accelerate the overall C to U-BS reaction. After the reaction the U-BS adduct was treated with a base, quantitatively converting U-BS to U (with MS of +1 compared with control, FIG. 2A). In addition to the much higher rate of the C-to-U conversion observed under the new BS conditions, the secondary structures in RNA fragments should also be fully denatured at 98° C. (e.g., if the incubation time is suitably long). It was hypothesized that the combination of these changes would dramatically reduce the false positives encountered regularly in conventional RNA m⁵C bisulfite sequencing.

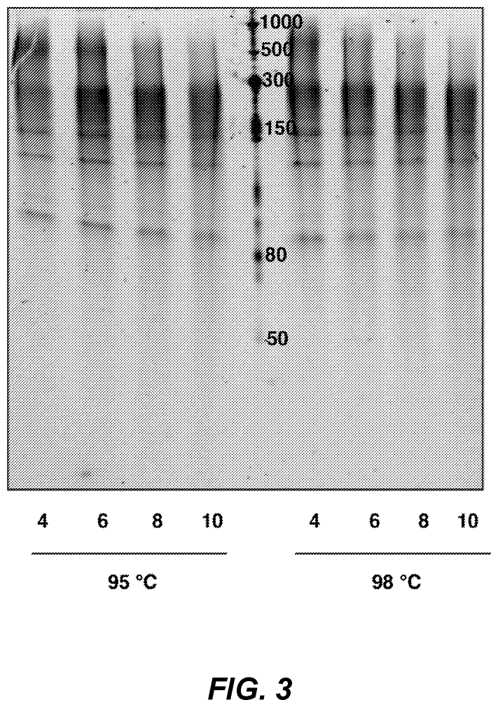

To investigate whether the new BS conditions might cause undesired m⁵C deamination, the corresponding m⁵C RNA oligonucleotide (SEQ ID NO: 2) was treated with the same conditions for different lengths of time. No reaction of m⁵C with BS was observed even after 30 minutes of treatment, suggesting that the new BS reaction does not generate false negatives at least within 30 min (FIG. 2B). To test whether the new BS conditions degraded RNA into too small of fragments, HeLa cell total RNA was processed using the R-1G BS recipe at 95° C. or 98° C. for different lengths of time and then a PAGE gel was run to evaluate the RNA fragment sizes. The majority of RNA fragments showed size distribution between 150-300 bp within 10 min of treatment (FIG. 3). The fragmented RNA with this size range can be used directly to build libraries with a random priming method, or can be further fragmented to smaller sizes (50 to 100 bp) to build libraries using a ligation-based method.

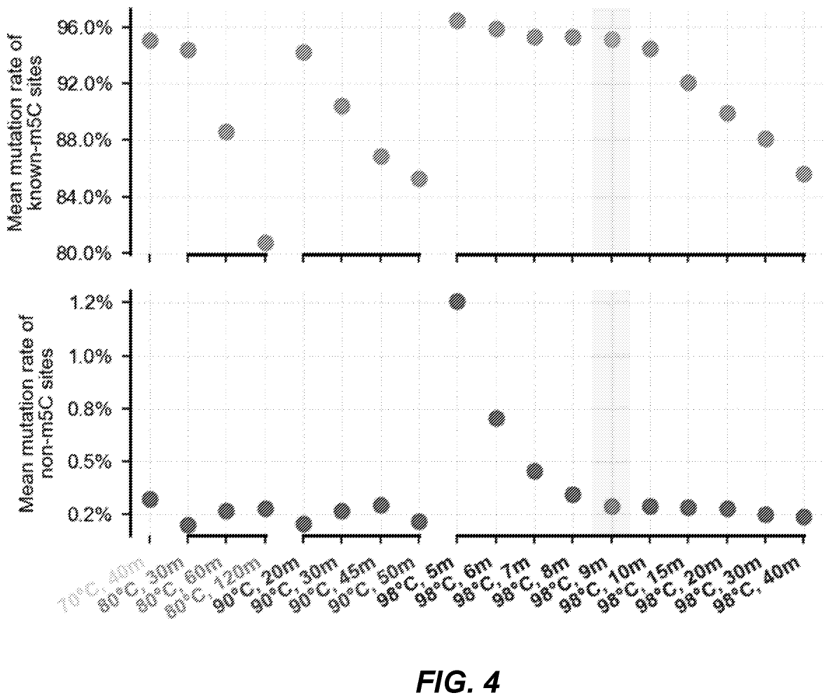

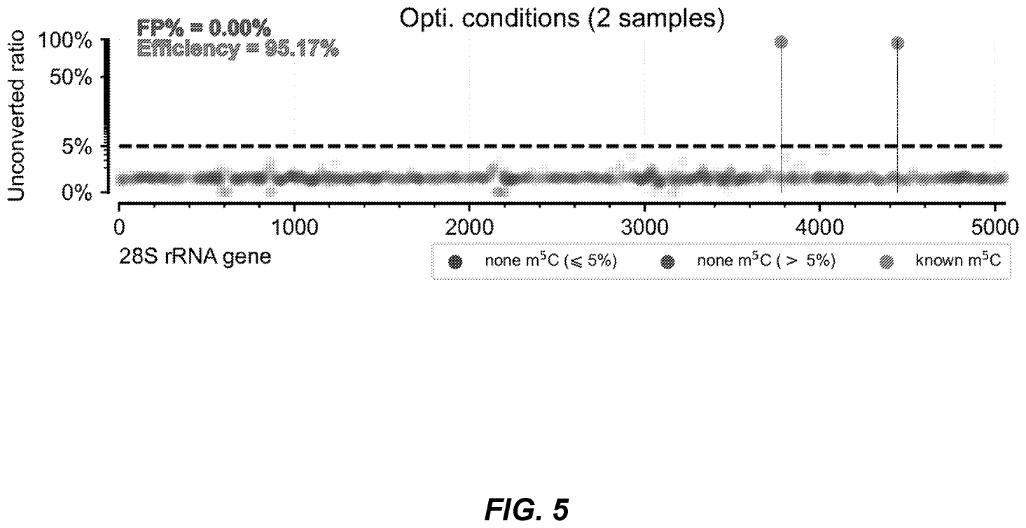

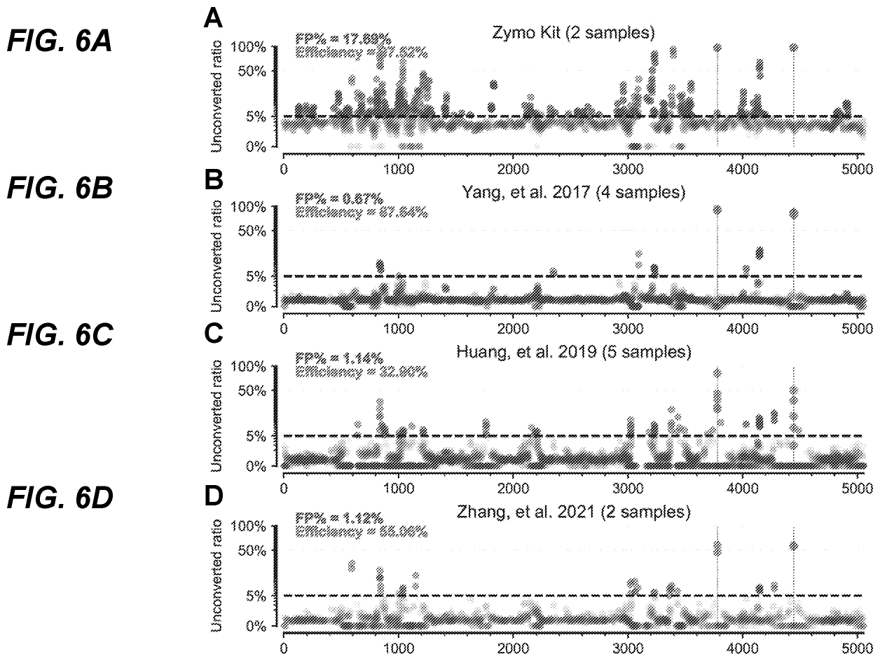

To validate that the new BS method can be used to build libraries with good read lengths and efficient C-to-U conversion without causing m⁵C deamination, and with reduced false positives caused by secondary structure, the method was applied to total RNA isolated from a range of different biological samples, including A549 cells. The individual biological sample total RNA was treated with recipe R1-G at a range of different temperatures and times, oligonucleotide libraries were constructed using NEB small RNA kit, and next generation sequencing was then performed. There are two confirmed m⁵C sites in human 28S RNA, while the other cytosine sites remain unmethylated. The studies sought to determine whether these two established m⁵C sites could be detected and whether m⁵C sites would show any undesired m⁵C deamination using the new BS method. In addition, human 28S rRNA contains rich secondary and tertiary structures and therefore conventional BS sequencing usually generates many false positives (e.g., incomplete conversion of unmethylated cytosine to uracil). As shown in FIG. 4, the undesired m⁵C deamination at the two known m⁵C sites increased with longer reaction time, while the background also decreased with longer time. Incubation conditions characteristic of certain improvements described herein were identified as incubation at 98° C. for 9 min, under which the average unconversion rate for the two known m⁵C sites were over 95%, while the unconversion rates were below 5% for all the C sites (FIG. 5). To facilitate direct comparisons, libraries were built using the new BS method side by side with EZ RNA Methylation Kit™ (Zymo Research), the most widely used kit to detect m⁵C in RNA, and the false positive rates were compared. As shown in FIG. 6A, the library prepared using the Zymo kit indeed detected the two known m⁵C sites (green dots with vertical lines noting their positions), however a large number of false positives (red dots) also appeared. A literature dataset was downloaded that was obtained using an “optimized” bisulfite condition (75° C. for 4 h)¹⁰. As shown in FIG. 6, false positives were indeed reduced relative to standard Zymo Kit protocols (false positive percentage (FP %) reduced from 17.60% to 0.67%), but some false positives remained, and the modification fractions of the two known m⁵C sites were also observably significantly decreased (efficiency of 97.52% reduced to 87.64%) (FIG. 6B). Two additional datasets were downloaded from the literature^{15,21}, in which the researchers used the Zymo BS reagent but conducted three cycles of BS treatment at higher temperature. The false positives were further reduced but there were still significant numbers of false positives detected (FIGS. 6C and 6D). In addition, serious undesired deamination on the two known m⁵C sites was also observed, indicating that all these literature BS conditions are not optimal.

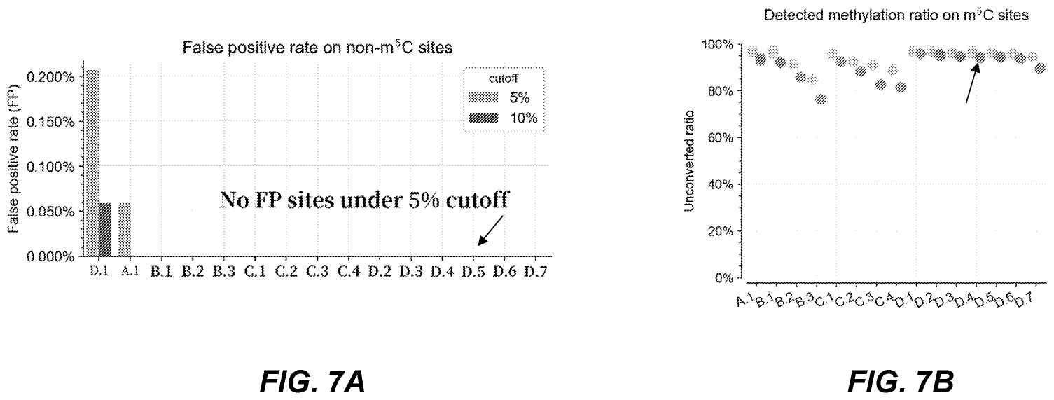

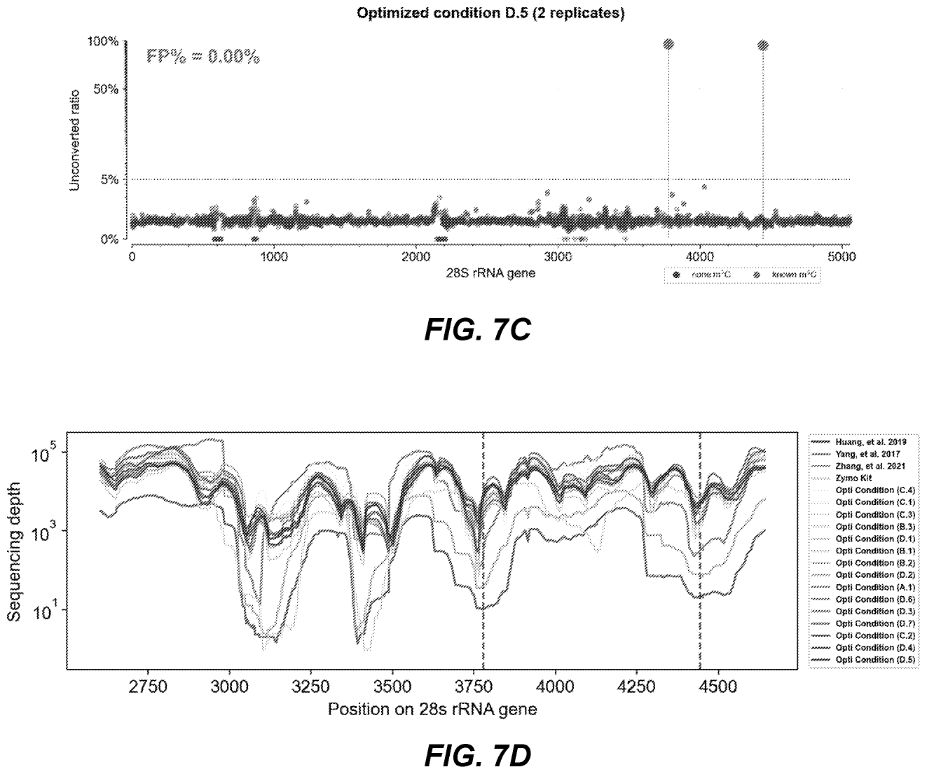

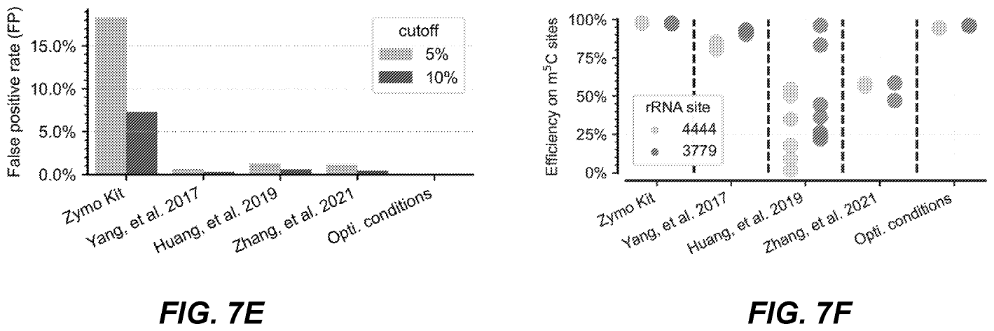

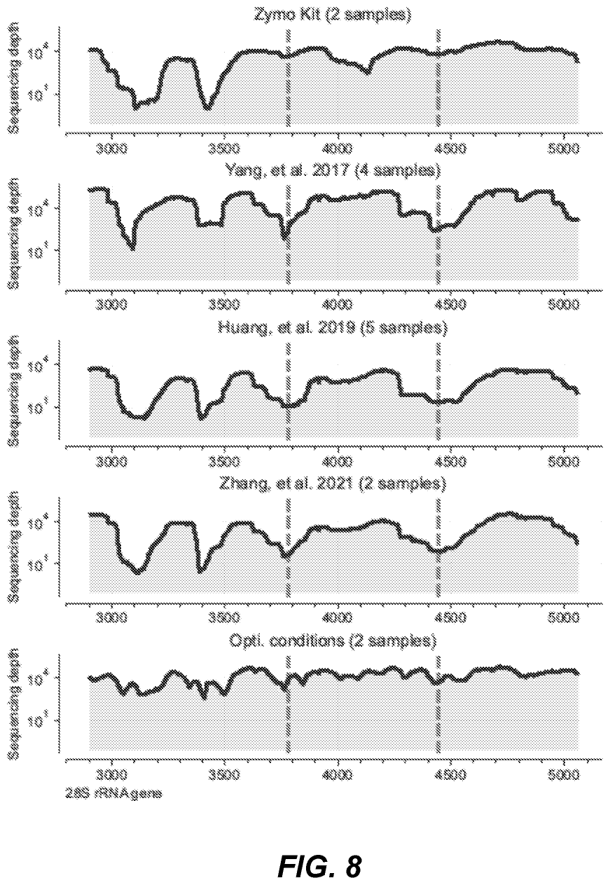

Statistical comparison of the BS treatments disclosed herein with canonical-BS treatments (e.g., Zymo-BS) and additional literature conditions were summarized in FIG. 7, the false positive rates using the BS treatment conditions disclosed herein were the lowest (FIG. 7E; “Opti conditions”), while the m⁵C signals detected at the two known sites using BS treatment conditions disclosed herein were the highest (FIG. 7F; “Opti conditions”). The inventors further compared the reads distribution patterns between the BS treatment conditions disclosed herein with other methods. Different lengths of incubation time and temperatures were screened using the R-1G recipe and it was found that in the vast majority of conditions, no false positive sites were detected when a 5% unconverted ratio cutoff was used (FIG. 7A). It was determined that the undesired m⁵C deamination rate increased with longer reaction times and higher temperature (FIG. 7B). Based on these observations, 98° C. incubation time for 9 min, using the R-1G recipe, gave conditions under which the two known msC sites were detected with very high msC fraction, while the false positive rate was zero when 5% unconverted ratio cutoff was used (FIG. 7C). These studies suggested that the new BS conditions solved the major challenges in BS sequencing of m⁵C in RNA. In addition, As shown in FIG. 7D and FIG. 8, the blue (FIG. 7D) black (FIG. 8) curves represented the reads distribution of our method and the red (FIG. 7D) curves and black curves (FIG. 8) represent several other literature methods. Those cytosine rich regions (e.g., 28S rRNA gene) showed sequencing coverage in all published data, the less depth regions contained more cytosines, causing more fragmentation, this was consistent with all the methods, suggesting that reacted cytosine in RNA causes RNA fragmentation, consistent with the proposed RNA fragmentation mechanism during BS treatment. However, the fluctuation of the read depth was much less using the disclosed method compared with the literature methods, suggesting that the new BS conditions generated much less RNA fragmentation and thus much less bias in estimating the m⁵C fraction.

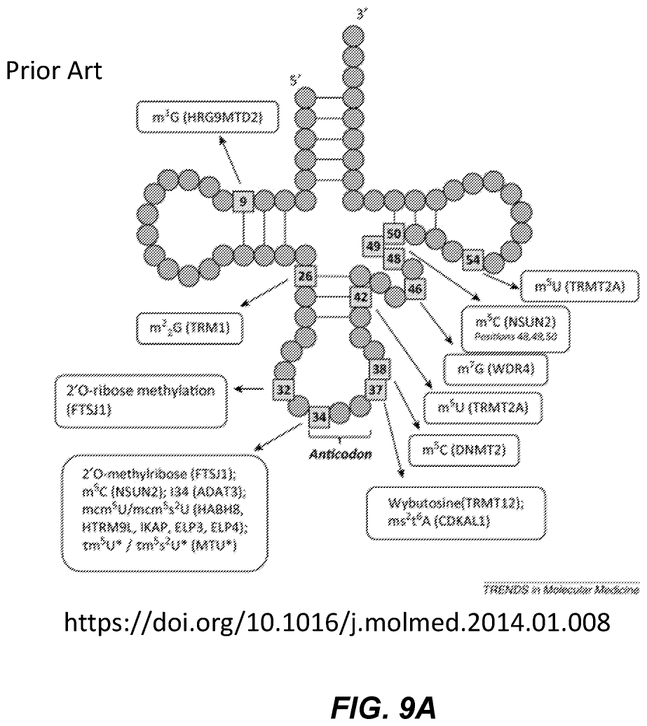

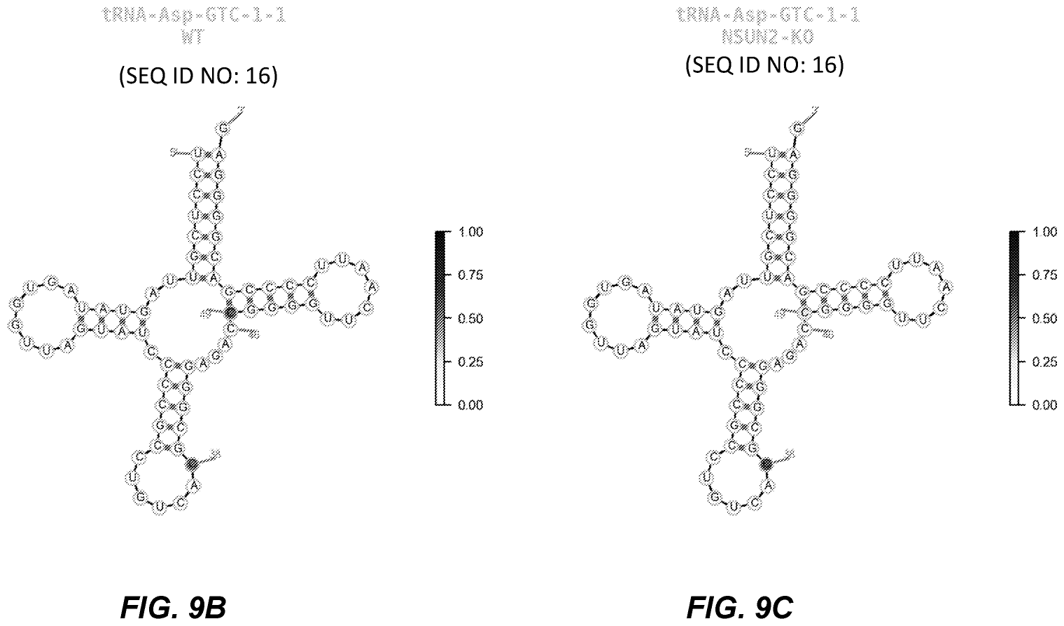

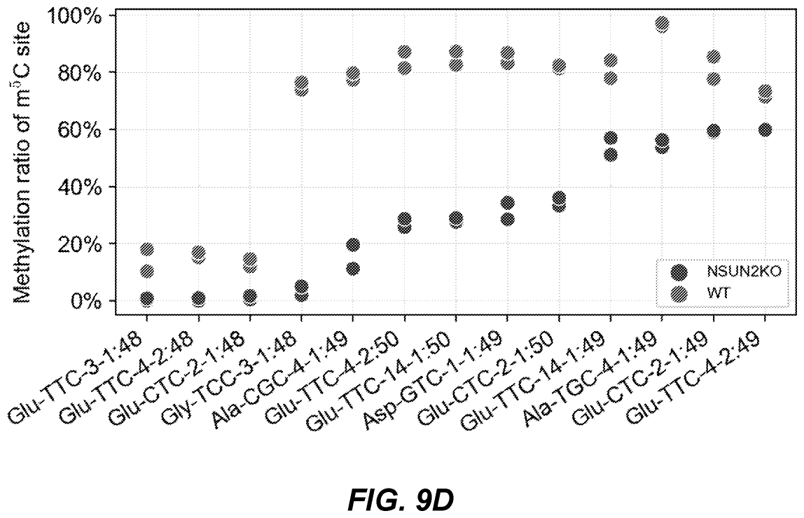

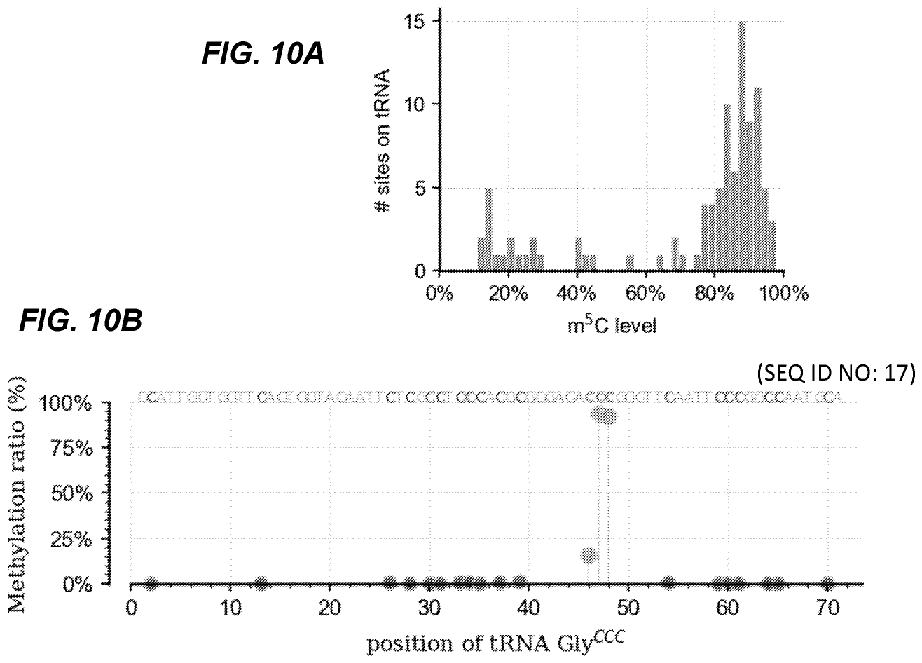

To further validate the disclosed methods, the small RNA fractions from wildtype A549 cell lines and its NSUN2 KO lines were sequenced. It is known that m⁵C is present at site 48, 49 or 50 in some tRNA species and they are substrates of NSUN2 methyltransferase (FIG. 9A). Therefore, it was expected that the m⁵C fraction at these sites should be sensitive to the NSUN2 knockout. In contrast, m⁵C site at C38 is the substrate of DNMT2 (FIG. 9A), and so its fraction should not be sensitive to the NSUN2 knockout. Indeed, as shown in FIGS. 9B-9D, the detected m⁵C fraction at site 48, 49 or 50 decreased significantly while the detected m⁵C fraction at C38 remained unchanged upon NSUN2 knockout, further confirming that the disclosed method is effective. Further analysis of the small RNA libraries showed that the majority of m⁵C sites detected in tRNA had high modification fractions (FIG. 10A). As an example, the unconverted rates at all the C and m⁵C sites in tRNA Gly^{CCC} was shown in FIG. 10B, all the C sites showed very low background, while two m⁵C sites at 49 and 50 showed very high modification fractions (>90%) while site 48 showed much lower modification fraction (<25%). The accurate and quantitative detection of these m⁵C sites in tRNA can facilitate study of the associated biological functions.

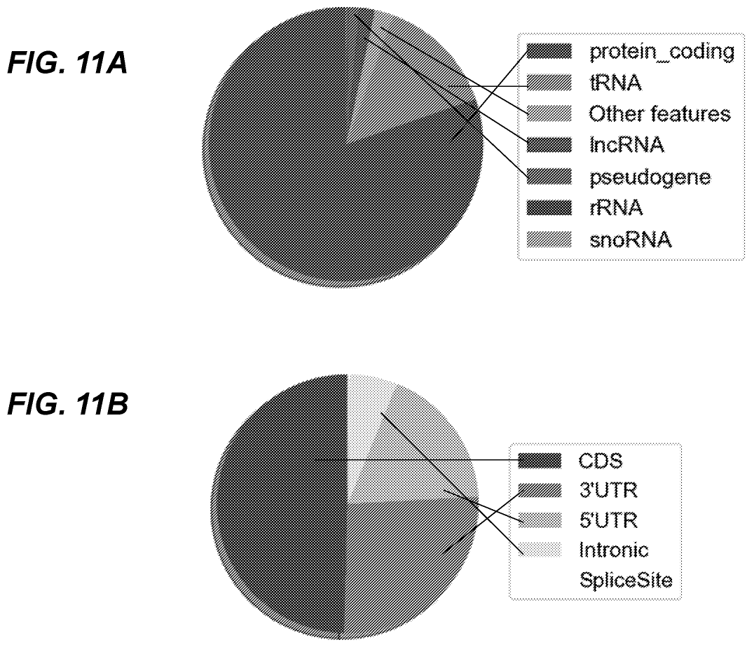

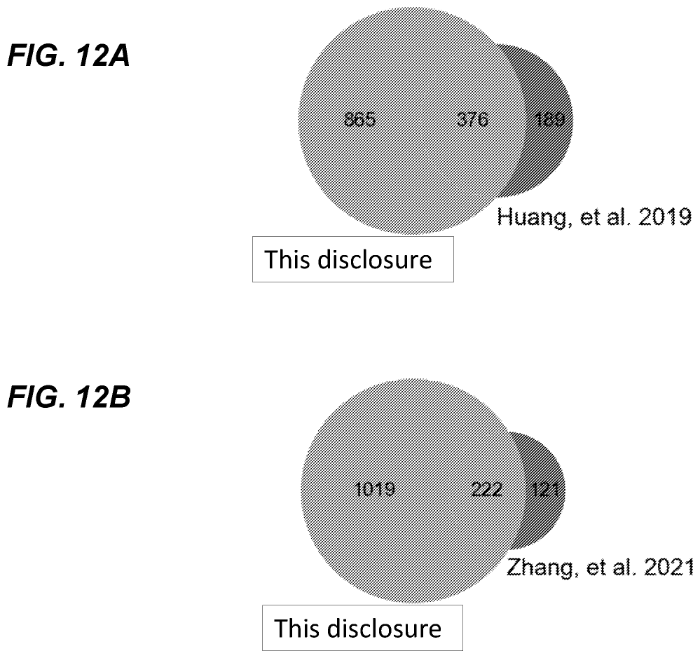

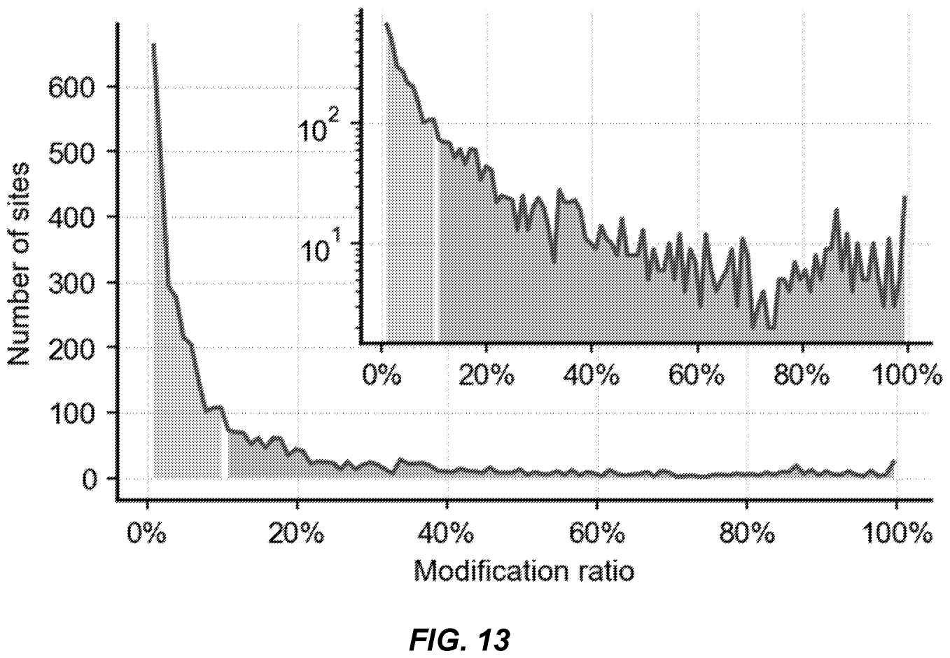

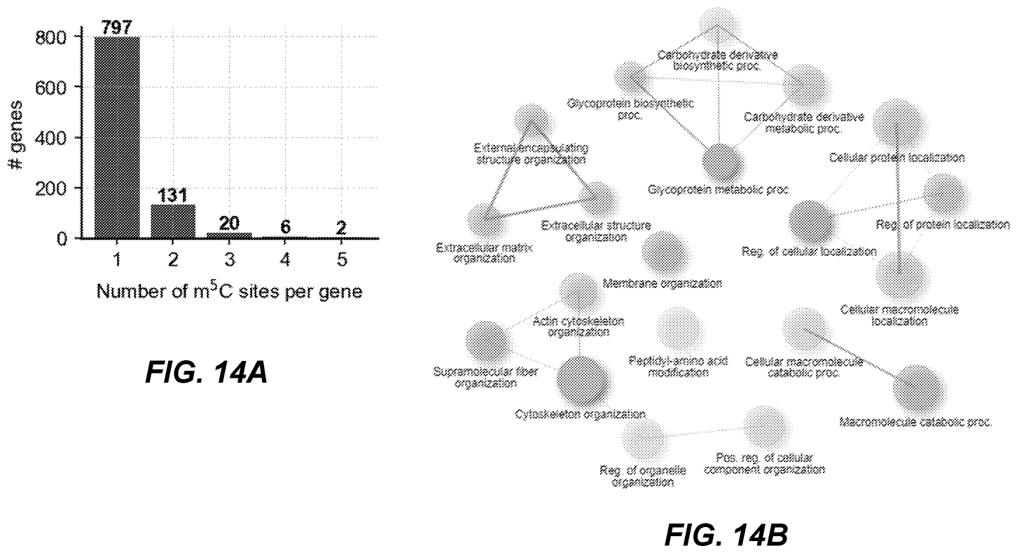

BS-seq protocols disclosed herein was then applied to HeLa mRNA. It was found that the majority of msC sites detected were located in protein-coding RNA (FIG. 11A), among which half of the sites were located in the coding sequence (CDS) region (FIG. 11B). Using the protocols described herein, the inventors were able to identify many more m⁵C sites when compared to two recent papers (Huang, et al., 2019, and Zhang, et al., 2021) (FIG. 12). For those m⁵C identified in Huang, et al.,¹⁵ and Zhang et al.,²¹ the majority were also found herein (e.g., 376/565 and 222/343 respectively), suggesting that the BS treatment methods described herein were accurate and more sensitive than methods previously described. The detected m⁵C sites had varied modification fractions ranging from 10-100%, with many sites being highly modified (FIG. 13). Most of genes containing m⁵C modification had only one m⁵C site, while 2 to 5 m⁵C sites were detected in 159 genes (FIG. 14A). Further gene ontology (GO) analysis showed that genes modified by m⁵C were involved in various gene functions, include glycoprotein metabolism, cytoskeleton organization, cellular localization etc. (FIG. 14B), suggesting that m⁵C modification in mRNA may have important biological functions.

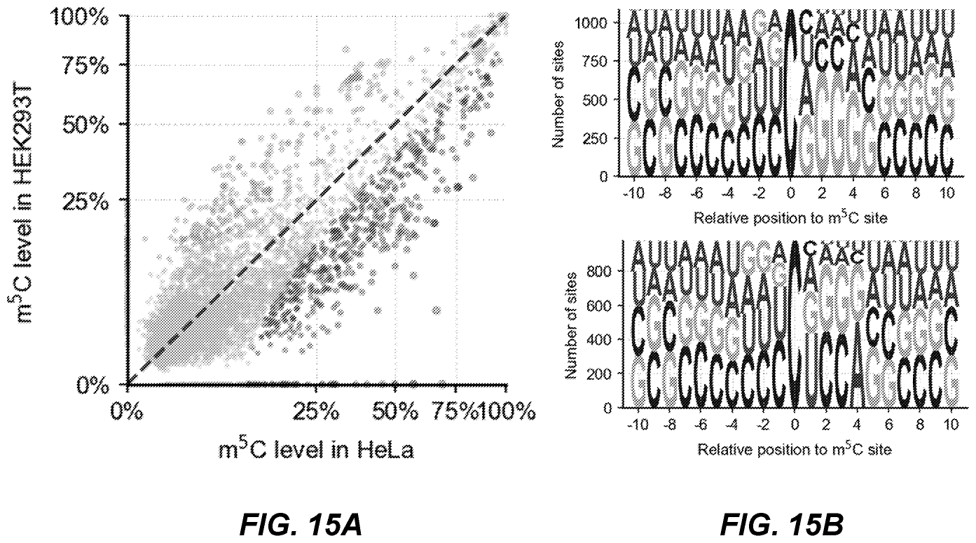

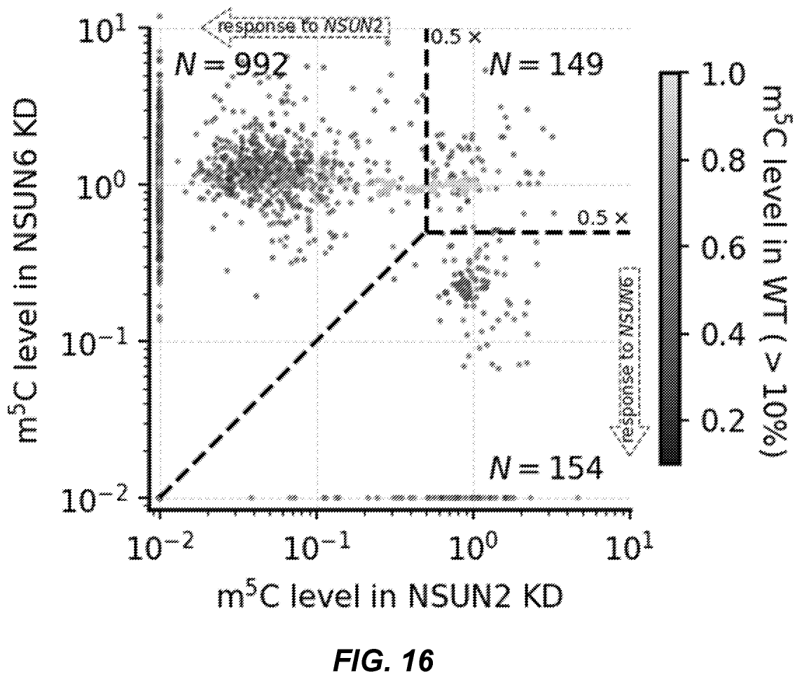

In addition to HeLa mRNA, the inventors also sequenced polyA+RNA extracted from HEK293T cells. As shown in FIG. 15A, the overall modification level of m⁵C sites was consistent between HeLa and HEK293T cell lines, however, there existed some differently modified sites. The m⁵C sites in HeLa cells showed more G-rich motifs, while m⁵C sites in HEK293T cells showed more CUCCA motifs (FIG. 15B). It has been reported that NSUN2 and NSUN6 are the methyltransferases depositing m⁵C on mRNA. Therefore, the inventors applied BS-seq protocols of the immediate disclosure to NSUN2 or NSUN6 knockdown HeLa cell mRNA extracts, and the corresponding shRNA control (FIG. 16). Sequencing results showed that more than ˜90% of the modified sites, mainly in G-rich motifs, dropped in NSUN2 knockdown cell mRNA extracts, suggesting that NSUN2 may play a major role in m⁵C modification in mRNA in HeLa cells. Additionally, the inventors also detected a small fraction of m⁵C sites, mainly in CUCCA motifs, that responded to NSUN6 knockdown. These results also suggest that the difference in modification profiles between cell lines may be associated with differential expression level of methyltransferases.

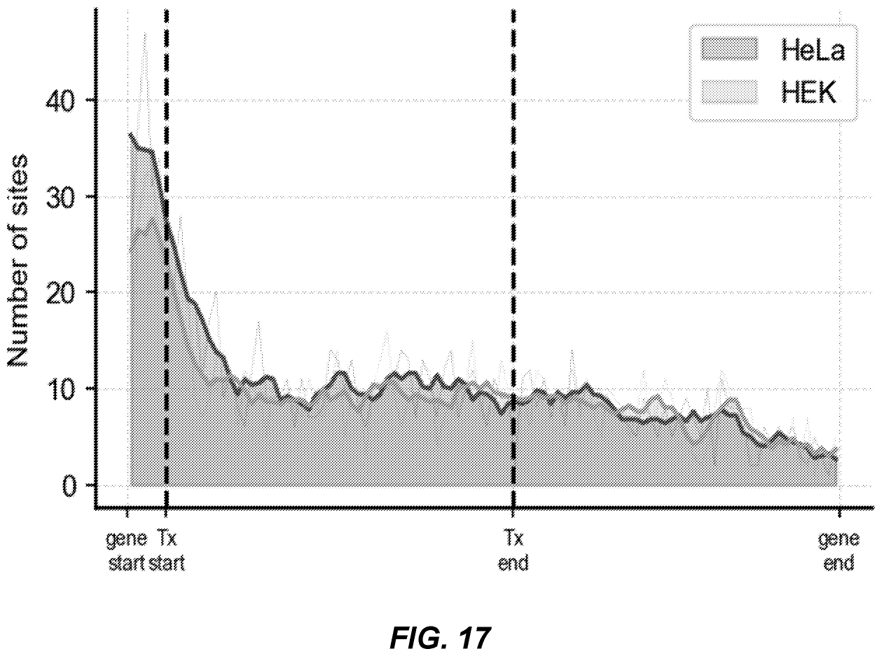

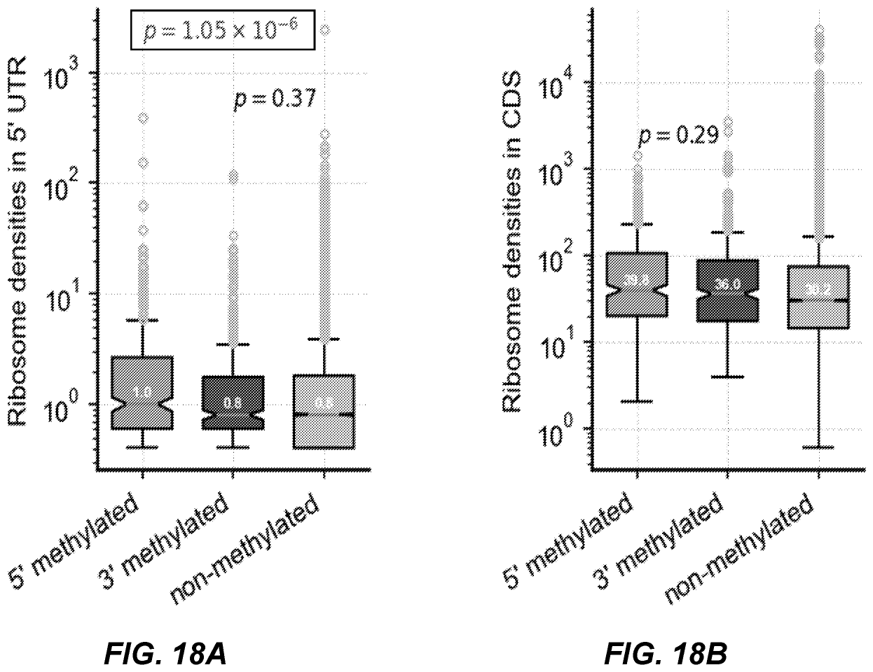

Interestingly, both HeLa and HEK293T cells showed similar enrichment patterns of m⁵C sites at the 5′-end of transcripts (FIG. 17). In connection with ribosome profiling data, the inventors found evidence that m⁵C modification at the 5′-end of transcripts may modulate translation efficiency. Compared to non-methylated genes, genes containing m⁵C sites at the 5′-end were enriched for ribosomal density signal at the 5′-UTR of the transcripts (p value=1.05×10⁻⁶), while genes containing m⁵C sites at the 3′-end did not show significant enrichment for ribosome density signal (p=0.37, FIG. 18A). Moreover, both 5′-end and 3′-end methylated genes did not show ribosome density enrichment within the CDS region (FIG. 18B).

### Experimental Procedures

### (1) Identification of BS Conditions Using RNA Model Oligos by Maldi-TOF MS

50% ammonium bisulfite and ammonium sulfite were mixed in different ratios to prepare different BS reagents. Then, 9 μL of BS reagent and mix with model RNA (AGCGA, 100 ng) (SEQ ID NO: 1) dissolved in water (1 μL). The mixture was incubated at different temperatures from 70-98° C. for different lengths of time, and the reaction was monitored by Maldi-TOF MALDI.

TABLE 1

| | 50% | | | | |
|---|---|---|---|---|---|
| | ammonium | Ammonium | | | Conversion rate |
| | bisulfite | sulfite | Temperature | Time | by Maldi-TOF |
| Entry | (μL) | (mg) | (° C.) | (min) | MS ( %) |
| 1 | 2000 | 498 | 70 | 60 | poor |
| 2 | 2000 | 701 | 70 | 60 | poor |
| 3 | 2000 | 999 | 70 | 60 | poor |
| 4 | 2000 | 1500 | 70 | 60 | poor |
| 5 | 343 | 1500 | 70 | 40 | 15% |
| 6 | 2000 | 300 | 70 | 10 | 90% |
| 7 | 2000 | 300 | 70 | 20 | 100% |
| 8 | 2000 | 300 | 70 | 40 | 100% |
| 9 | 2000 | 300 | 80 | 10 | quantitative |
| 10 | 2000 | 300 | 90 | 10 | quantitative |
| 11 | 1000 | 170 | 70 | 5 | ~80% |
| 12 | 1000 | 160 | 70 | 5 | ~80% |
| 13 | 1000 | 150 | 70 | 5 | ~80% |
| 14 | 1000 | 140 | 70 | 5 | ~80% |
| 15 | 1000 | 130 | 70 | 5 | ~90% |
| 16 | 1000 | 120 | 70 | 5 | ~90% |
| 17 | 1000 | 100 | 70 | 5 | Near quantitative |
| 18 | 1000 | 90 | 70 | 5 | ~90% |
| 19 | 1000 | 80 | 70 | 5 | ~90% |
| 20 | 1000 | 70 | 70 | 5 | ~85% |
| 21 | 1000 | 60 | 70 | 5 | ~90% |
| 22 | 1000 | 50 | 70 | 5 | ~85% |
| 23 | 1000 | 100 | 98 | 1 | 80% |
| 24 | 1000 | 100 | 98 | 2 | Near quantitative |
| 25 | 1000 | 100 | 98 | 3 | quantitative |
| 26 | 1000 | 100 | 98 | 4 | quantitative |
| 27 | 1000 | 100 | 98 | 5 | quantitative |

It was found that the most efficient condition was use of BS reagent R-1G (the solution of 1 mL 50% ammonium bisulfite and 100 mg ammonium sulfite) and a reaction mixture incubation at 98° C., converting C to U-BS adduct quantitatively within 3 min. Further alkaline treatment converted U-BS to U, resulting in complete C-to-U conversion. Under the same condition using a similar model RNA (AGm⁵CGA) (SEQ ID NO: 2) as substrate, Maldi-TOF-MS showed that there was no msC-BS adduct formation within 30 min, suggesting that this new BS condition was highly selective and did not generate false negatives.

(2) Analysis of BS Conditions by Sequencing 28S rRNA from HeLa Cells Using R-1G

Incubation temperatures from 70 to 90° C. were tested with incubation time from 20 to 40 min with or without adding urea. The average cytosine conversion rates are all higher than 98% and the two msC sites both gave over 90% fraction. There was no obvious benefit by adding urea.

TABLE 2

| | Urea | Temperature | Time | | Average cytosine |
|---|---|---|---|---|---|
| Entry | (M) | (° C.) | (min) | m⁵C fractions | conversion rate (%) |
| 1 | 0 | 70 | 40 | 96.5%/92.6% | >98% |
| 2 | 1 | 70 | 40 | 96.9%/92.0% | >98% |
| 3 | 0 | 80 | 30 | 96%/92.6% | >98% |
| 4 | 1 | 80 | 30 | 97.2%/91.5% | >98% |
| 5 | 1 | 90 | 20 | 95.8%/92.4% | >99% |

Additional conditions were tested using R-1G. It was found that treatment at 98° C. gave the highest cytosine conversion efficiency.

TABLE 3

| | Temperature | | | Average cytosine |
|---|---|---|---|---|
| Entry | (° C.) | Time (min) | m⁵C fractions | conversion rate (%) |
| 1 | 80 | 60 | ~95% | >99.5% |
| 2 | 80 | 120 | ~80% | >99.2% |
| 3 | 90 | 30 | ~90% | >99.2% |
| 4 | 90 | 45 | ~87% | >99.5% |
| 5 | 90 | 60 | ~84% | >99.7% |
| 6 | 98 | 5 | ~96% | >99.2% |
| 7 | 98 | 10 | ~94% | >99.7% |
| 8 | 98 | 15 | ~92% | >99.7% |
| 9 | 98 | 20 | ~90% | >99.7% |
| 10 | 98 | 30 | ~85% | >99.7% |
| 11 | 98 | 40 | ~83% | >99.7% |

After identifying 98° C. as the temperature giving the highest cytosine conversion efficiency, various reaction times were tested and it was found that 9 min gave the highest cytosine conversion efficiency (99.7%) and detected high m⁵C fraction (94.5%) (see Table 4 below).

TABLE 4

| | Temperature | | | Average cytosine |
|---|---|---|---|---|
| Entry | (° C.) | Time (min) | m⁵C fractions | conversion rate (%) |
| 1 | 98 | 5 | ~96% | >99.2% |
| 2 | 98 | 6 | ~95% | >99.4% |
| 3 | 98 | 7 | ~95% | >99.5% |
| 4 | 98 | 8 | ~95% | >99.6% |
| 5 | 98 | 9 | ~94.5% | >99.7% |
| 6 | 98 | 10 | ~94% | >99.7% |

### (3) Studies of Treatment Time and Temperature Using BS Reagent R-1G and A549 Total RNA by Next-Generation Sequencing

The mixture of 9 μL BS reagent R-1G with 1 μL A549 total RNA (200 ng) was incubated at 70-98° C. for different lengths of time, and then 140 μL water was added. In-column desulphonation was conducted by following canonical-BS treatment instructions (e.g., Zymo EZ RNA Methylation™ Kit instructions). The RNA was further treated with 0.1 M NaHCO₃ at 95° C. for 3 min to fragment to size of 50-80 nt. After OCC purification and 3′-repairing and 5′-phosphorylation using T4 PNK, the RNA fragments were further purified by OCC and eluted with 7 μL water. 6 μL was used to build libraries using NEB small RNA libraries kit and the libraries were sequenced by Nova-seq. After data analysis, it was determined that incubation at 98° C. for 9 min was a condition under which the false positives are all removed and the two known m⁵C sites in 28S rRNA showed high fractions. Libraries were also built starting from the same amount of A549 total RNA (200 ng) side by side using canonical-BS treatments (e.g., EZ RNA Methylation™ Kit from Zymo research). Sequencing results showed that the two known m⁵C sites showed high m⁵C fraction, but many false positive sites also showed up, suggesting many unconverted cytosine sites.

### Example 2—m⁵C Detection and Analysis in RNA from Low Input Samples

For patient samples, such as blood or embryo samples, the amount of sample available is usually limited (such as 10˜100 ng total RNA). For this small amount of total RNA, it is not realistic to use poly-T beads to enrich mRNA or use ribo-minus to deplete rRNA to get enough RNA samples to build libraries with good quality using the ligation-based methods. Therefore, short DNA probes are added to RNA obtained from a low-input sample (e.g., blood sample, single cell RNA) to anneal with rRNA and then RNase H is added to digest rRNA to small fragments. After purifying the undigested other RNA with paramagnetic beads, RNA is subjected to BS treatment using the R-1G bisulfite reagent at 98° C. for 9 min, followed by random priming to synthesize cDNA, and then a ssDNA library construction kit is used to build libraries. msC sites are detected in non-rRNA of low-input total RNA samples.

### Example 3—Bisulfite Sequencing for 5mC Detection and Analysis in DNA

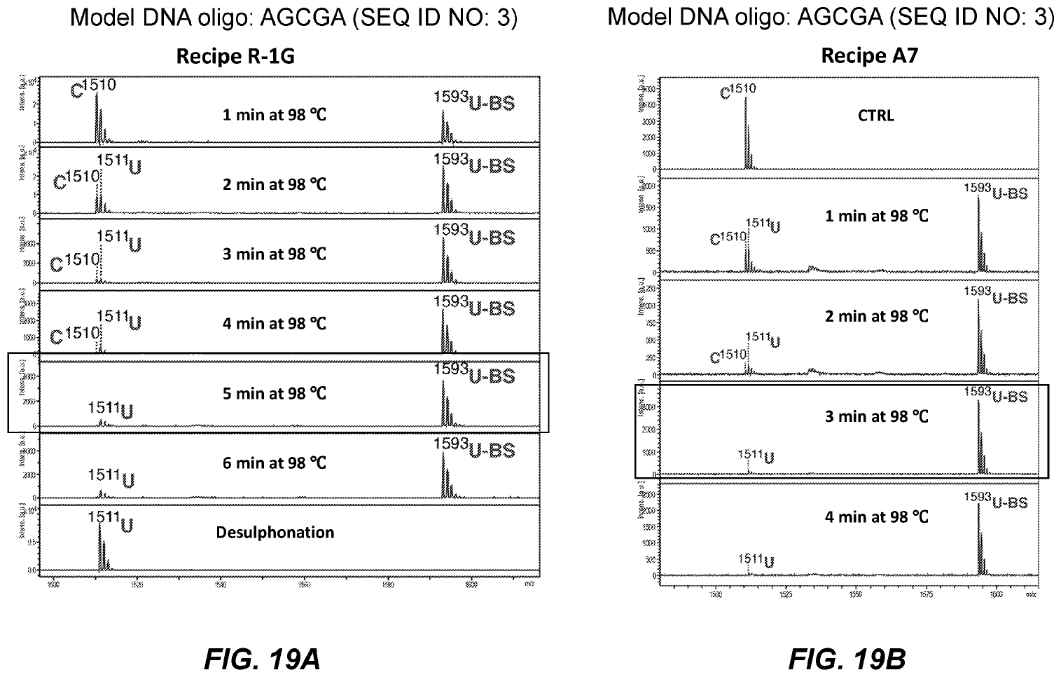

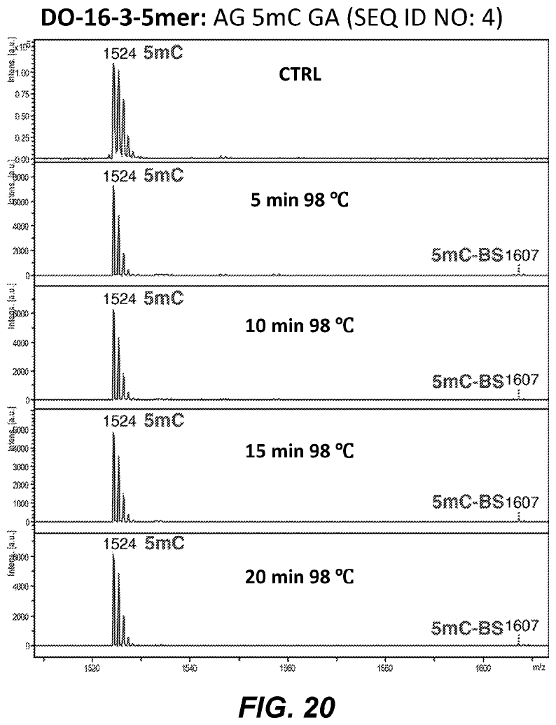

Initially, bisulfite conversion was tested on DNA using the R-1G BS recipe applied to DNA oligonucleotide AGCGA (SEQ ID NO: 3). The reaction was observed to be slower than observed for the RNA oligonucleotide, and needed 5 minutes at 98° C. to complete (FIG. 19A). Additional recipes were screened and a new recipe was identified, termed A7 (1 mL 70% ammonium bisulfite+100 μL 50% ammonium bisulfite; bisulfite concentration ˜9.5 M, pH ˜5.1), which converted dC-to-dU quantitatively within 3 minutes (FIG. 19B). No obvious 5mC deamination was observed within 10 minutes of treatment (FIG. 20).

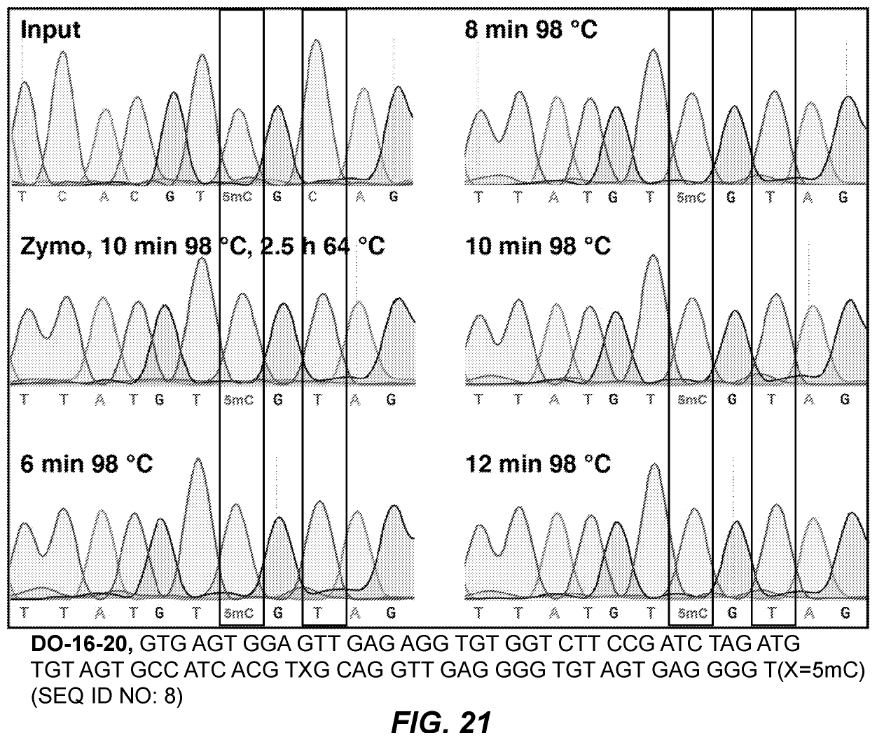

When a 82mer synthetic DNA oligo containing both C and 5mC (SEQ ID NO: 8); was treated with BS recipe A7 at 98° C. for 4 to 10 min, Sanger sequencing results showed that 5mC was read as C in all cases, while C was read as T quantitatively after 8-12 min (FIG. 21), confirming that this recipe was not only highly efficient in inducing C-to-U conversion in a very short time, but also highly selective, avoiding undesired 5mC deamination.

(DO-16-20)
SEQ ID NO: 8
GTGAGTGGAGTTGAGAGGTGTGGTCTTCCGATCTAGATGT
GTAGTGCCATCACGT5mCGCAGGTTGAGGGGTGTAGTGAG
GGGT (SEQ ID NO: 8)

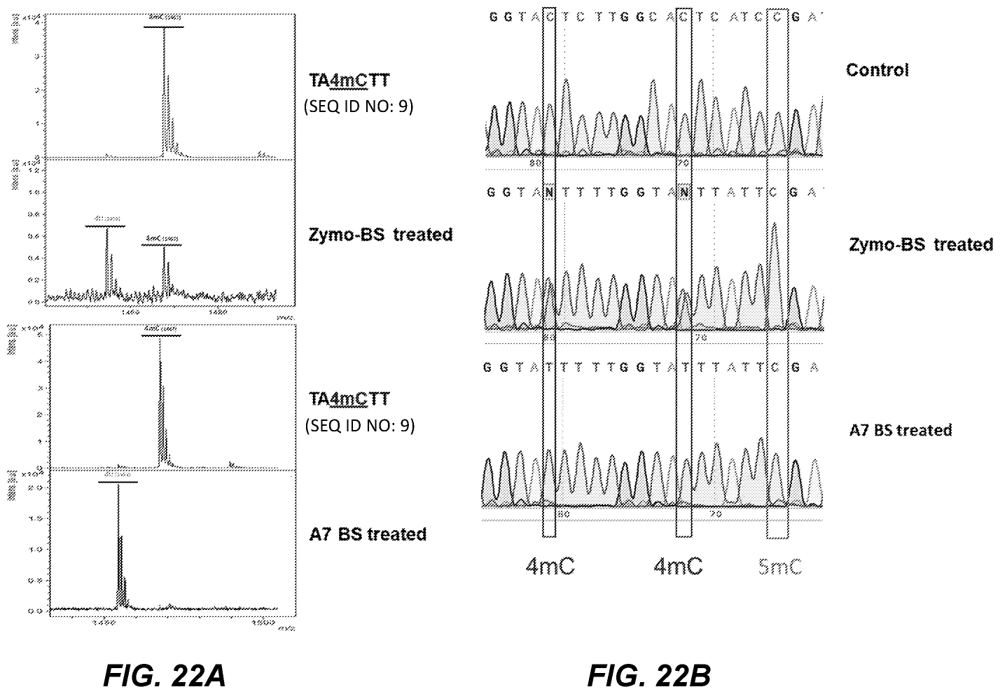

For DNA BS sequencing, it can also be important to distinguish 5mC from 4mC. 4mC has been known to exist in bacteria, and recently was detected in eukaryote genomic DNA²⁷. Previously the inventors found that canonical-BS treatments (e.g., Zymo BS conditions) can only deaminate 4mC with ˜50% efficiency²⁸, and thus the 4mC sites may result in false positives 5mC detection sites when using BS sequencing. The inventors reasoned that the reaction conditions disclosed herein, including higher temperatures and BS recipe concentrations, could facilitate the deamination of 4mC. To test this, short DNA oligos containing a 4mC modification (TA4mCTT; SEQ ID NO: 9) were treated with BS conditions of the disclosure, side by side with canonical-BS treatments (e.g., Zymo BS conditions). Maldi TOF MS data showed that when canonical-BS treatments were utilized, 4mC was partially deaminated to give the corresponding oligo containing dU with around 50% efficiency, while conversely when utilizing new BS conditions disclosed herein, 4mC was quantitatively converted to dU (FIG. 22A). To test the deamination efficiency of C, 5mC, and 4mC using BS conditions disclosed herein compared to previously disclosed canonical-BS treatments (e.g., Zymo kit BS treatments), a synthetic 100 bp DNA oligo containing C and both 5mC and 4mC modifications (SEQ ID NO: 12) was synthesized and treated with BS conditions disclosed herein or canonical-BS treatments (e.g., Zymo BS conditions). Following treatment, Sanger sequencing was conducted to evaluate the deamination efficiency of C, 5mC and 4mC. The results showed that after incubation with BS reagent of the immediate disclosure at 98° C. for 10 min, 4mC and C sites were all quantitatively read as T while the 5mC site remained to be read as C (FIG. 22B), in contrast, when canonical-BS treatment (e.g., Zymo kit BS treatment) was utilized, the two 4mC sites were both read as C and T in a 1:1 ratio. These results suggest that BS conditions of the immediate disclosure can avoid, in certain cases completely, the false positives generated by the existence of 4mC in the genome, in sharp contrast to previously disclosed canonical-BS treatments.

SEQ ID NO: 12
GTGAGTGGAGTTGAGAGGTGTGGTGGTA4mCTCTTGGCA4mCT
CATC5mCGATCACGTAGATGTGTAGTGCCATCACGTCGCAGGT
TGAGGGGTGTAGTGAGGGGT (SEQ ID NO: 12)

DNA degradation is a known problem in BS sequencing. It not only causes DNA material loss which is a more serious problem in low-input DNA samples, but may also cause biased cleavage of DNA so that the 5mC fraction detected could be over-estimated²⁷. Based on the suggested DNA degradation mechanism in traditional BS treatment¹⁹, C-BS adduct formed in BS treatment is the main species causing deglycosylation to form an AP site, leading to further DNA backbone cleavage via β-elimination. While 5mC does not react with BS, C sites will be much more prone to be cleaved than 5mC. Therefore, the BS treatment will cause more severe DNA damage in the C-enriched DNA sequences and thus the DNA fragments containing richer C will be less represented in the libraries, leading to over-estimation of 5mC level.

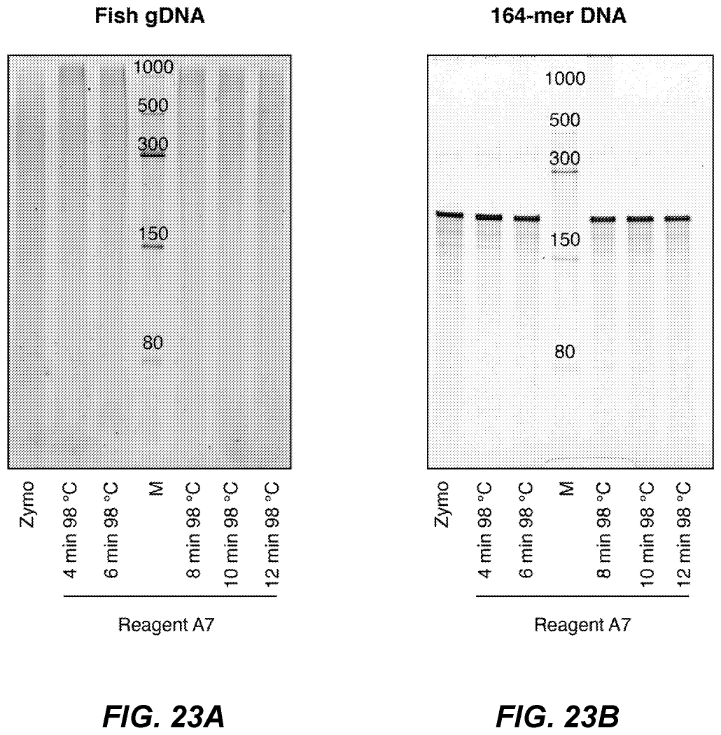

Since several recipes disclosed herein, including A7, were found to be highly efficient at converting C-BS to U-BS adduct, using these recipes and the disclosed conditions this deamination step was no longer the rate-limiting step, and thus C-BS adduct was only present in the reaction for a very short time and in very low concentration. Therefore, it was expected that the DNA damage would be significantly reduced using the disclosed recipes (including A7) and treatment conditions compared with other BS conditions. Even though the very high BS concentration and very high temperature used to accelerate BS reaction in the disclosed methods may hypothetically accelerate DNA degradation, it was hypothesized that the much shorter reaction time would outperform the acceleration of DNA degradation caused by high temperature and high BS concentration. To test this hypothesis, fish gDNA and synthetic 164mer dsDNA (mimicking the size of cfDNA; (SEQ ID NO: 13, and anti-sense SEQ ID NO: 14) were treated with BS recipe A7 for different time periods and compared side by side with canonical-BS treatment (e.g., Zymo EZ DNA Methylation-Gold® Kit). As shown in FIGS. 23A-23B, in both cases, it was observed that within 4-10 min, recipe A7 (1 mL 70% ammonium bisulfite+100 μL 50% ammonium bisulfite) caused less DNA damage compared to the canonical-BS treatment (e.g., Zymo kit, 98° C. for 10 min followed by 64° C. for 2.5 hrs), suggesting that it has the potential to be applied to low input DNA and may overcome the issue of over estimation of 5mC fraction.

SEQ ID NO: 13
GTGAGTGGAGTTGAGAGGTGTGGTACGGTGACTCAGGTTTGTGCT
CTTCCGATCTAGATGTGTAGTGCCATCCGAT5mCGCATATGCGAG
TCACGTACATGCTACTGTCAGTACTGATGGACCTTTCT5mCGCAG
TGGCGACTATGGTTGAGGGGTGTAGTGAGGGGT;
(SEQ ID NO: 13)
SEQ ID NO: 14
ACCCCTCACTACACCCCTCAACCATAGTCGCCACTG5mCGAGAAA
GGTCCATCAGTACTGACAGTAGCATGTA5mCGTGACTCGCATATG
CGATCGGATGGCACTACACATCTAGATCGGAAGAGCACAAACCTG
AGTCACCGTACCACACCTCTCAACTCCACTCAC
(SEQ ID NO: 14)

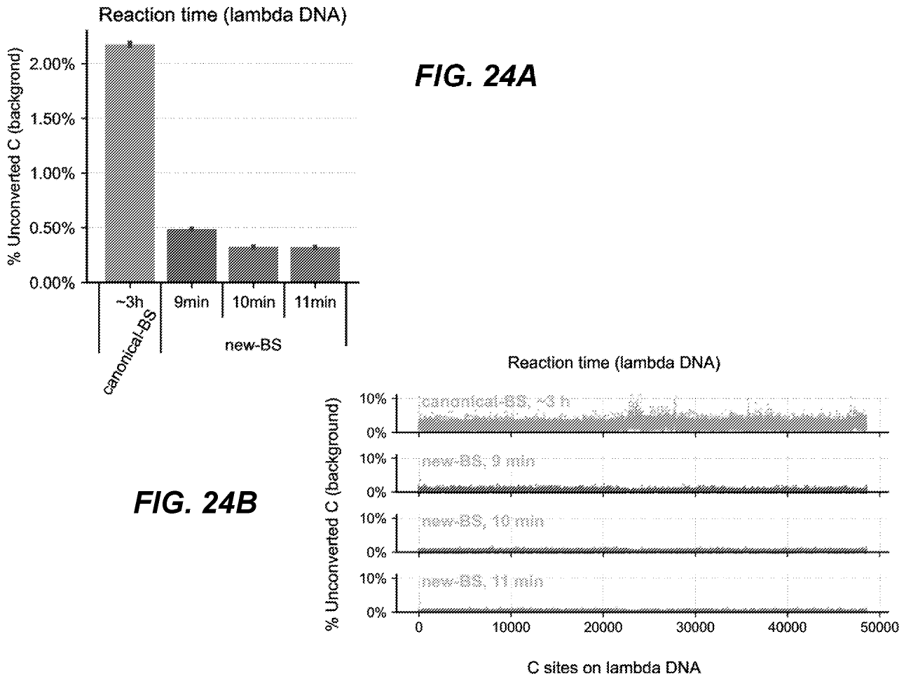

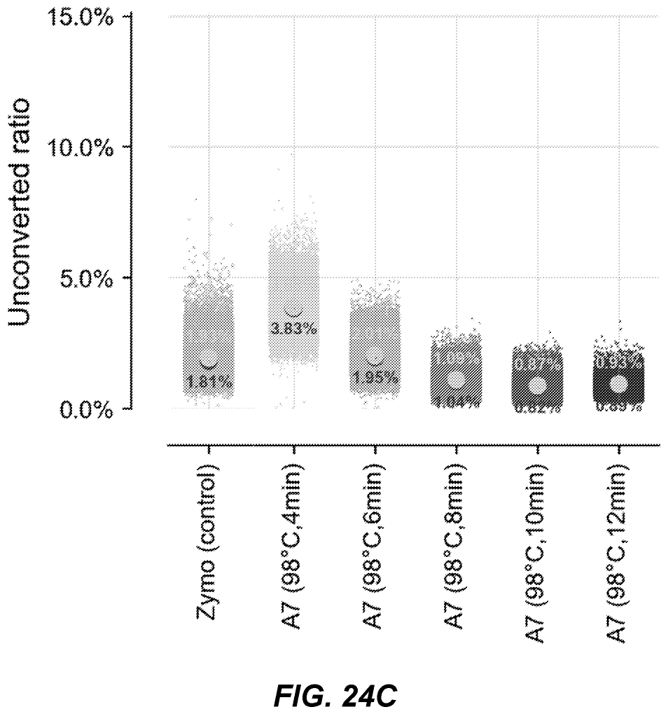

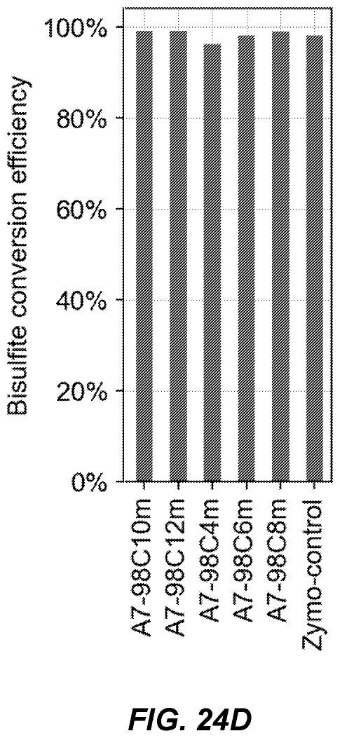

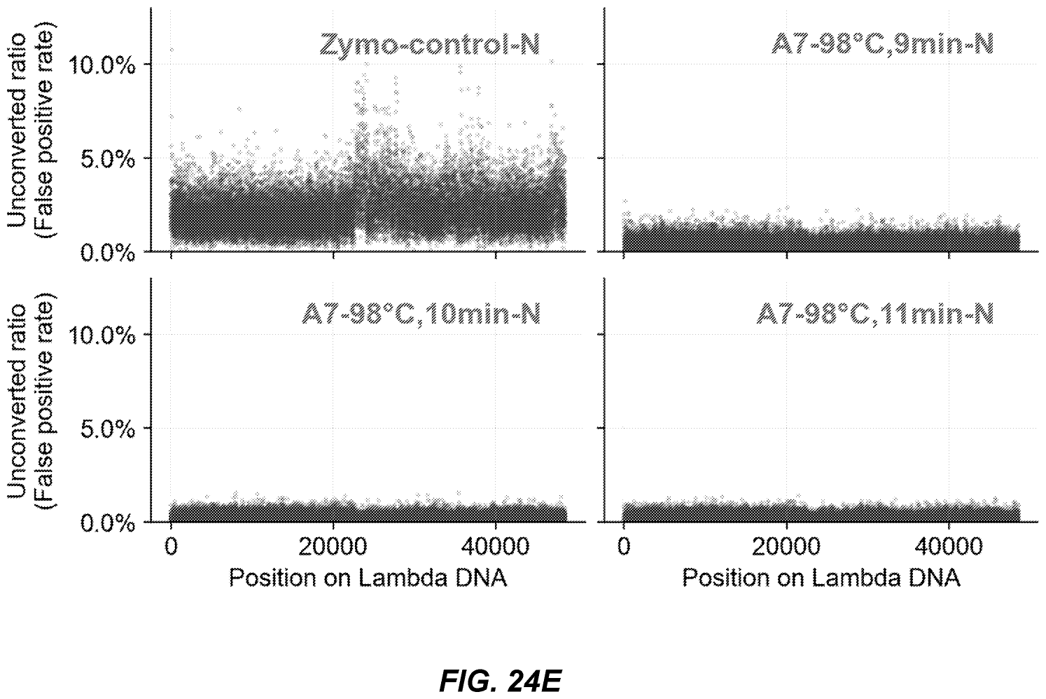

With the above established principle, the inventors proceeded to evaluate the new method using biological DNA containing synthetic spike-ins. Given that canonical-BS treatment options, like the Zymo EZ DNA Methylation-Gold® Kit, is the standard for BS sequencing, libraries were built side by side to make a direct comparison. gDNA of Arabidopsis thaliana has a small genome (approximately 135 mega bases) which was previously reported to have 5mC sites, and so was chosen as exemplary gDNA for these studies. In order to compare the conversion efficiency of all the C sites, spike-in lambda DNA containing no 5mC sites was added to evaluate the background. Spike-in synthetic 164mer dsDNA containing four 5mC sites (SEQ ID NO: 13, and anti-sense SEQ ID NO: 14) was also added to evaluate the undesired 5mC demethylation rate. After BS treatment and libraries construction with Swift Accel-NGS Methyl-Seq DNA Library Kit (single-stranded DNA library construction) and NGS sequencing, sequencing data showed that background was the lowest after incubation time reached 10 minutes, and that for all the C sites in lambda DNA, the average C-to-U conversion rate reached greater than or equal to about 99.2% (the average unconverted rate was 0.82% as shown in FIG. 24C, additional assays resulted in 99.6% conversion rate with 0.4% unconverted rate, as shown in FIG. 24A) for recipe A7 after 10 min reaction. In comparison, the average conversion rate was 98.2% for canonical-BS treatments (e.g., Zymo kit, the average unconverted rate was 1.81% as shown in FIG. 24C, additional assays resulted in average conversion rates of 97.8% with an unconverted rate of 2.2%, as shown in FIG. 24A) (FIG. 24C and FIG. 24A respectively), indicating that the disclosed BS recipe and conditions (“new-BS”) gave higher conversion efficiency (FIGS. 24A-24E). Importantly, the unconverted rate for each C site showed much larger fluctuation using the canonical-BS treatments (e.g., Zymo kits), which required high cutoff (10%) to avoid false positives, while in the new-BS recipe and conditions, the unconverted rate at each site was more homogenous, and almost all sites showed unconverted rate below 2% (FIGS. 24B and 24E).

### (1) Identification of BS Conditions Using DNA Model Oligos by Maldi-TOF MS

70% ammonium bisulfite and 50% ammonium bisulfite was mixed in different ratios to prepare different BS reagents. Then take 9 μL BS reagent and mix with model DNA (AGCGA, 100 ng) dissolved in water (1 μL). The mixture was incubated at different temperatures at 98° C. for different lengths of time, and the reaction were monitored by Maldi-TOF MALDI. See Table 5 and Table 6 below.

TABLE 5

| | 70% | 50% | | | Conversion rate |
|---|---|---|---|---|---|
| | ammonium | ammonium | Temperature | Time | by Maldi-TOF |
| Entry | bisulfite (μL) | bisulfite (μL) | (° C.) | (min) | MS (%) |
| 1 | 1000 | 0 | 70 | 3 | ~80% |
| 2 | 1000 | 50 | 70 | 3 | ~75% |
| 3 | 1000 | 100 | 70 | 3 | ~90% |
| 4 | 1000 | 150 | 70 | 3 | ~80% |
| 5 | 1000 | 200 | 70 | 3 | ~80% |

TABLE 6

| | 70% | 50% | | | Conversion rate |
|---|---|---|---|---|---|
| | ammonium | ammonium | Temperature | Time | by Maldi- |
| Entry | bisulfite (μL) | bisulfite (μL) | (° C.) | (min) | TOF MS (%) |
| 1 | 1000 | 100 | 98 | 1 | 70% |
| 2 | 1000 | 100 | 98 | 2 | Near quantitative |
| 3 | 1000 | 100 | 98 | 3 | Quantitative |
| 4 | 1000 | 100 | 98 | 4 | Quantitative |
| 5 | 1000 | 100 | 98 | 5 | Quantitative |
| 6 | 1000 | 100 | 98 | 6 | Quantitative |

It was found that the best results were obtained using BS reagent A7 (mixture of 1 mL 70% ammonium bisulfite and 100 μL 50% ammonium bisulfite) and incubating the reaction mixture at 98° C., converting C to U-BS adduct quantitatively within 3 min. Further alkaline treatment converted U-BS to U, resulting in complete C-to-U conversion. Under the same conditions using a similar model DNA (AG5mCGA; SEQ ID NO: 4) as substrate, Maldi-TOF-MS showed that there was almost no T-BS adduct formation within 20 min, indicating that the new BS conditions are highly selective.

(2) Using BS Reagent A7 to Treat Plant gDNA Containing Lambda DNA and a Synthetic 164 nt DNA Oligo Containing Two 5mC Sites by Next-Generation Sequencing

A mixture of 9 μL BS reagent A7 with 1 μL plant gDNA (50 ng) containing 0.5 ng lambda DNA without 5mC modification and 0.1 ng synthetic 164mer dsDNA containing four 5mC sites (SEQ ID NO: 13, and anti-sense SEQ ID NO: 14) was incubated at 98° C. for different lengths of time, and then 140 μL water was added. In-column desulphonation was conducted by following canonical-BS treatments (Zymo EZ DNA Methylation-Gold® Kit), eluting with 7 μL water. 6 μL was used to build libraries using Swift Accel-NGS Methyl-Seq DNA Library Kit and the libraries were sequenced by Nova-seq. After data analysis, it was determined that incubation at 98° C. for 10 min was the best condition, under which the average conversion efficiency of all the cytosine sites in lambda DNA was 99.18% (see Table 7 below). For the two known 5mC sites in 164 bp spike-in DNA, the detected fractions were above 96%. Side by side libraries were also built starting from the same amount of DNA using canonical-BS treatments (e.g., Zymo EZ DNA Methylation-Gold® Kit), and sequencing results showed that the average conversion efficiency of all the cytosine sites in lambda DNA was 98.2% using canonical-BS treatment. For the two known 5mC sites in 164 nt spike-in DNA, the detected fractions were 98% using canonical-BS treatment.

TABLE 7

| | | Temperature | | Average cytosine |
|---|---|---|---|---|
| Entry | Reagent | (° C.) | Time (min) | conversion rate (%) |
| 1 | A7 | 98 | 4 | >96.17% |
| 2 | | 98 | 6 | >98% |
| 3 | | 98 | 8 | >98.96% |
| 4 | | 98 | 10 | >99.18% |
| 5 | | 98 | 12 | >99.1% |

### Example 4—5mC Detection and Analysis in DNA from Low Input Samples

Libraries are built using Swift kit coupled with the new BS treatment conditions using A7 recipe, starting from 0.1, 1.0 or 10 ng mouse embryonic stem cell (mES) genomic DNA (gDNA) or from 0.1, 1.0 or 10 ng human cell-free DNA (cfDNA). Sequencing results are analyzed to identify methylation sites in the DNA.

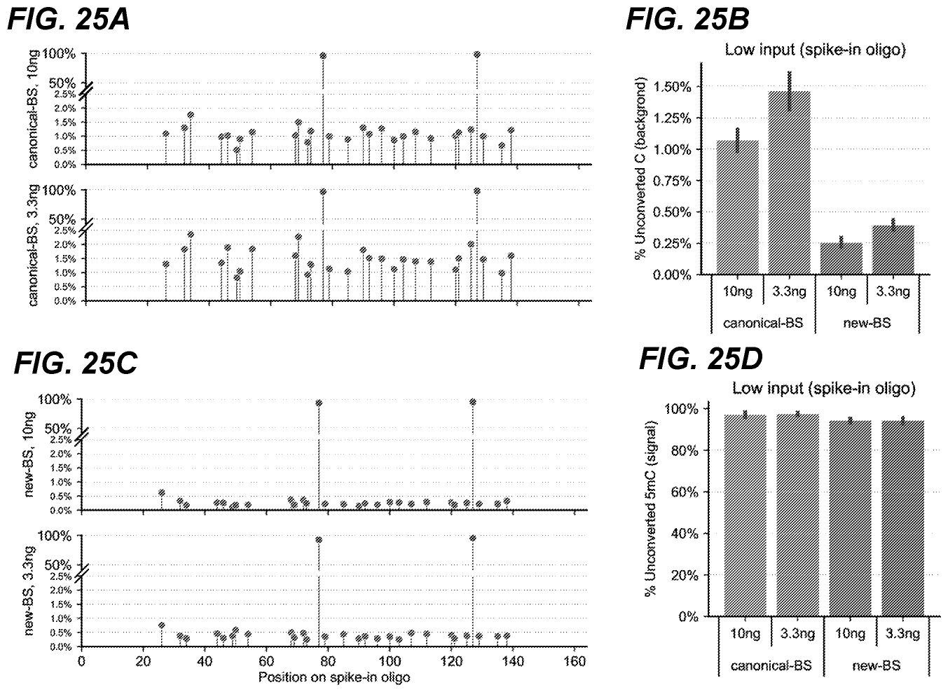

The inventors applied BS protocols disclosed herein (e.g., recipe A7 and incubation at 98° C. for 10 min) to mouse embryonic stem cell (mESC) gDNA. As the recipes and conditions disclosed herein generated less DNA damage than conventional BS conditions, the inventors reasoned that these protocols could be utilized for assays with low input gDNA. To evaluate conversion efficiency, gDNA sequencing libraries treated with the BS protocols disclosed herein were generated, libraries were generated with starting concentrations of 10 ng or 3.3 ng mESC gDNA, and lambda DNA with no 5mC sites spiked in. Additionally, synthetic dsDNA containing 5mC was also spiked-in to evaluate undesired 5mC conversion rates. To facilitate direct comparison of the disclosed protocols to current conventional BS protocols, side-by-side libraries were also generated using canonical-BS treatments (e.g., Zymo EZ DNA Methylation-Gold® Kit). After sequencing, the inventors analyzed the conversion rate of all C sites and the two known 5mC sites in the synthetic dsDNA. As showed in FIG. 25, the two libraries starting from 3.3 ng mESC gDNA gave slightly higher background than those starting from 10 ng (FIG. 25B). When comparing the two libraries generated using the BS protocols disclosed herein to canonical-BS treatments (FIG. 25C compared to FIG. 25A), the new BS protocols gave much lower background. On the other hand, the undesired conversion rate for 5mC in all 4 libraries are low (FIG. 25D)

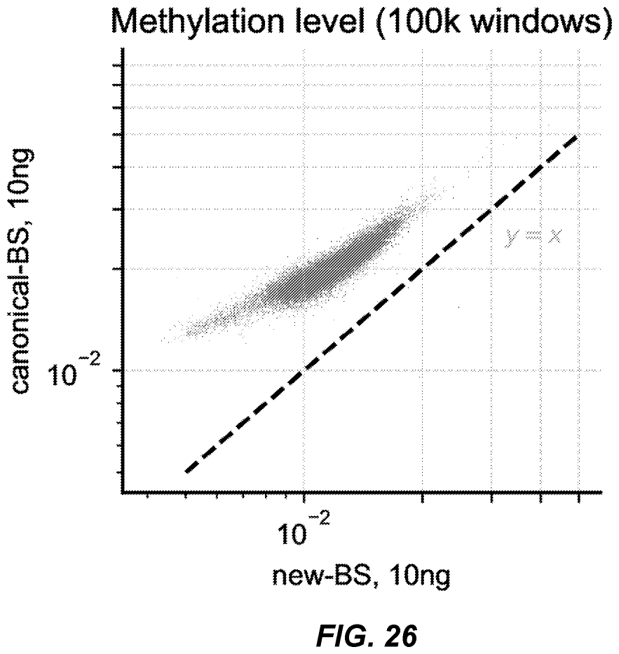

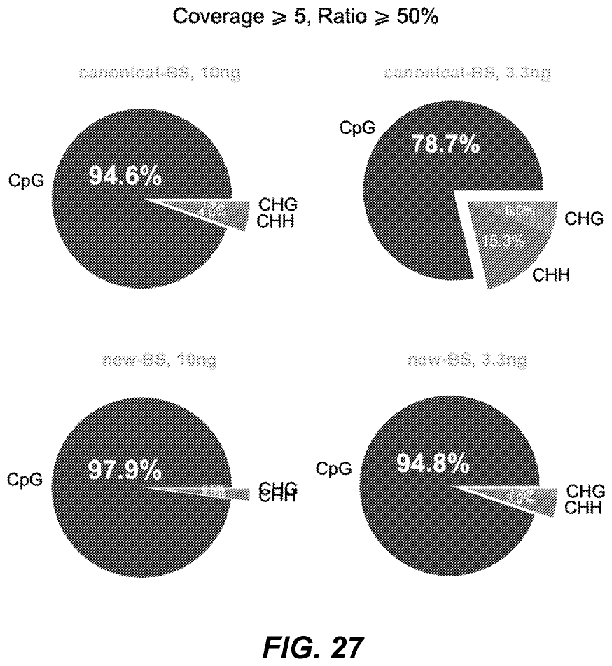

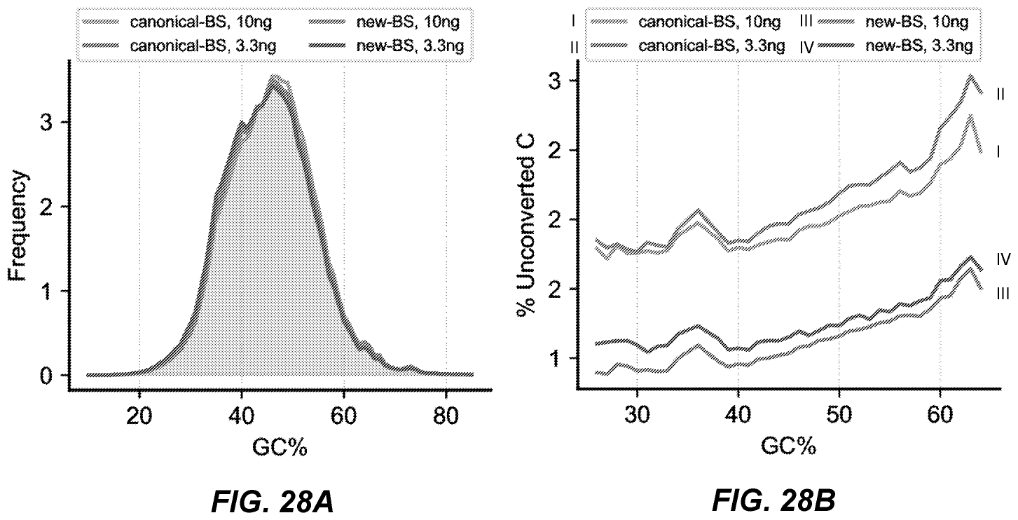

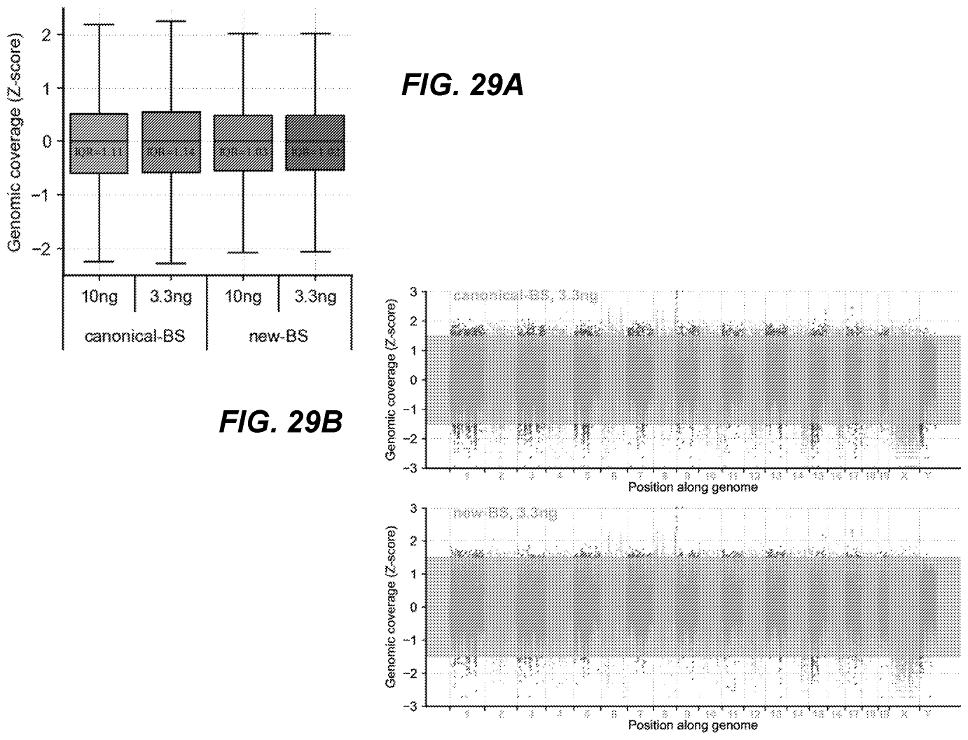

In addition, methylation levels detected from sequencing libraries generated with canonical-BS treatment systematically showed higher ratios than the BS treatment protocols disclosed herein (FIG. 26). This may be due to higher background noise levels of canonical-BS treatments when compared to the BS protocols disclosed herein. Studies using canonical-BS treatments might over-estimate the methylation levels in the genome. Meanwhile, canonical-BS treatment data reported more methylated sites in non-CpG motifs (FIG. 27), which may also be a consequence of relatively increased levels of background noise when compared to the protocols disclosed herein. Background noise is random signal and has more chance to be found in non-CpG sites. When studying non-CpG methylation, increased background can misinform investigators regarding biological significances, and potentially and lead to erroneous conclusions. Samples treated with BS protocols disclosed herein showed similar genomic coverage at different GC % regions when compared to canonical-BS treated samples (FIG. 28A). But canonical-BS treated samples showed higher fractions of unconverted C, especially at high GC % regions (FIG. 28B). Furthermore, the two libraries generated using the BS protocols disclosed herein also showed more evenly distributed genomic coverage than those generated using canonical-BS treatment (FIGS. 29A and 29B), demonstrating an additional advantage of the methods and compositions disclosed herein when compared to canonical-BS treatments.

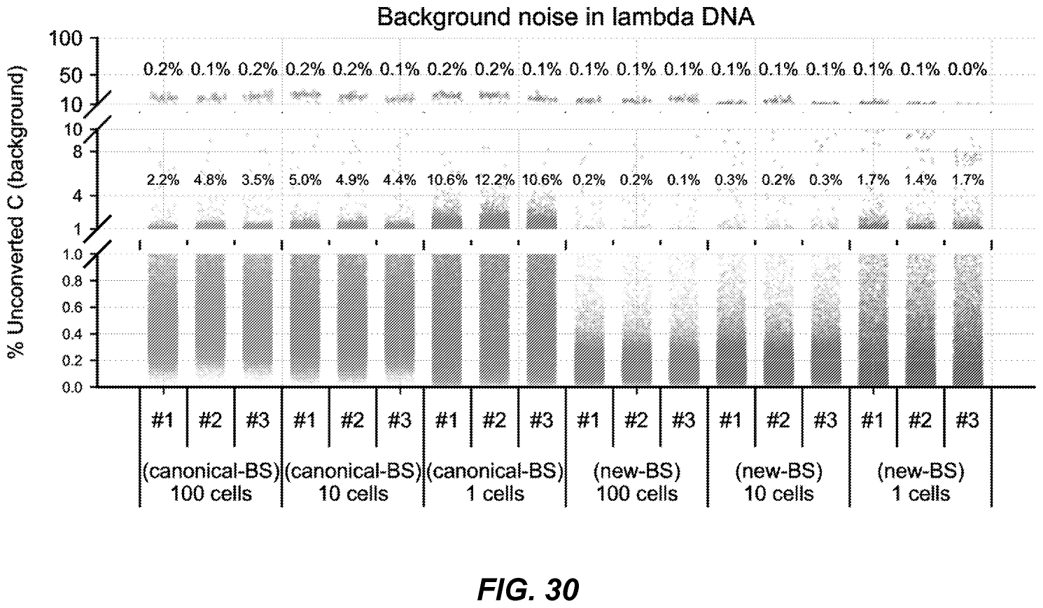

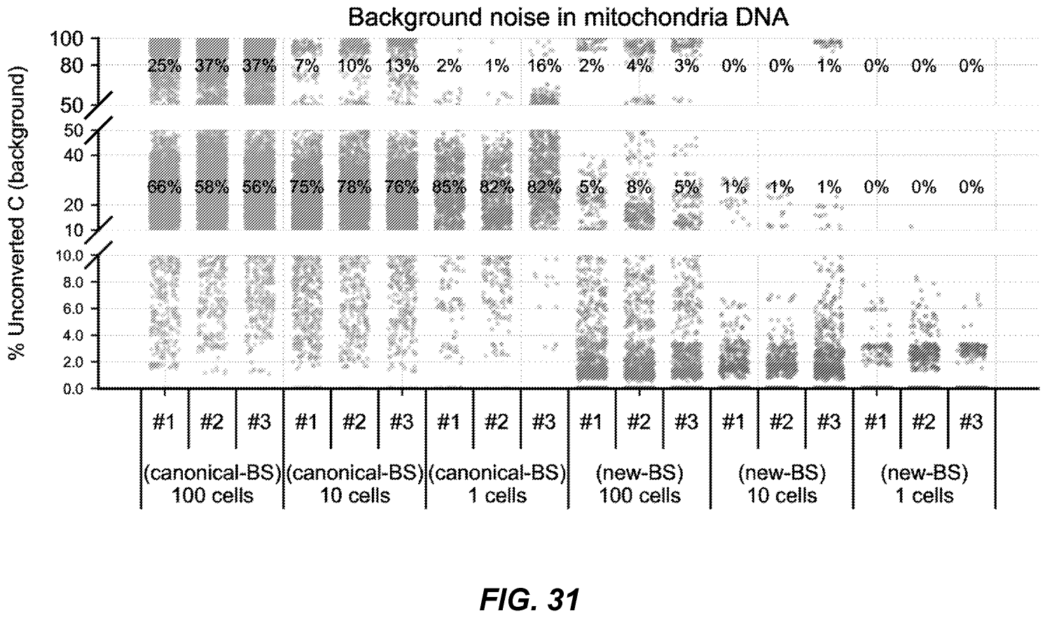

BS protocols described herein were utilized to generate ultralow or low gDNA input libraries created using mES cells (1, 10 and 100 cells respectively) with spike-in lambda DNA. The BS conversion efficiency from both lambda DNA and mitochondria DNA (mtDNA) were evaluated, as all the cytosine sites were free of 5mC modification. As shown in FIG. 30 and FIG. 31, unconverted C background noise decreased when input amount increased. For example, 1 cell samples showed higher background than 10 cell samples, while 10 cell samples showed higher background than 100 cell samples. The BS protocols described herein resulted in much lower levels of background when compared to canonical-BS treatment. For lambda DNA, when 1% was set as the cutoff for background, canonical-BS treatment showed more than 10 times the levels of false positives (e.g., % unconverted C) relative to the BS protocols disclosed herein (e.g., an average of ˜4.9% vs. ˜0.36% for the three 10 cell sample trials, FIG. 30). For highly structured mitochondria DNA (which in general had a higher background levels than lambda DNA, potentially due to the highly structured nature of mtDNA and challenges associated with achieving high conversion levels due to incomplete denaturation), when 10% was set as the cutoff for background, the false positive ratios using the canonical-BS treatment were more than 80 times higher than the BS protocols disclosed herein (e.g., an average of ˜86.3% vs. ˜1.3% for the three 10 cell sample trials, FIG. 31). These results showed that the BS treatment protocols disclosed herein were superior to the canonical-BS treatment protocols when ultralow input gDNA was utilized.

### Example 5˜5hmC Detection and Analysis in DNA

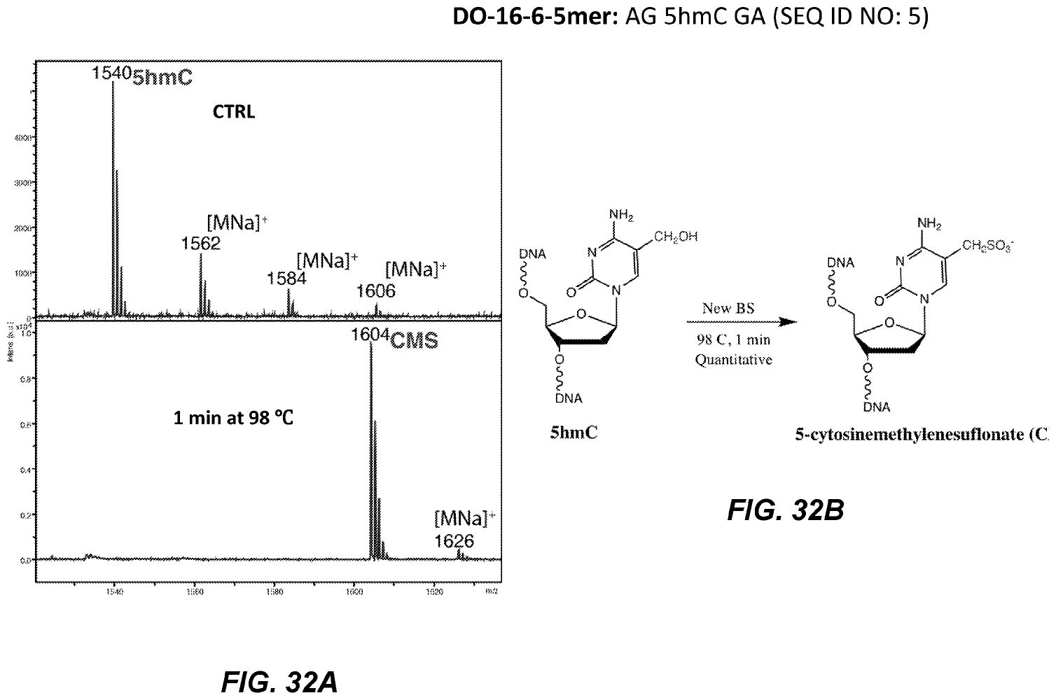

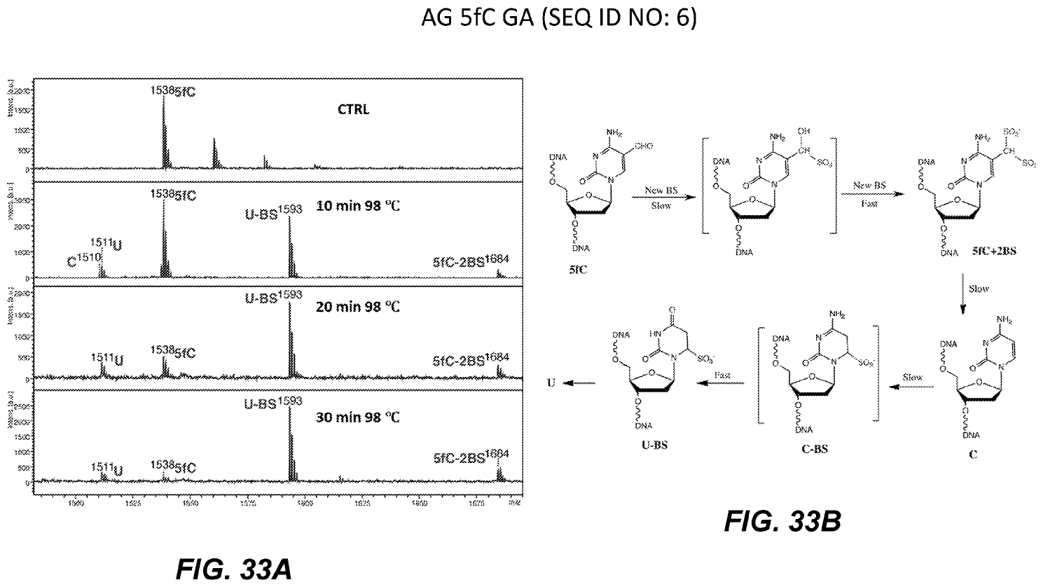

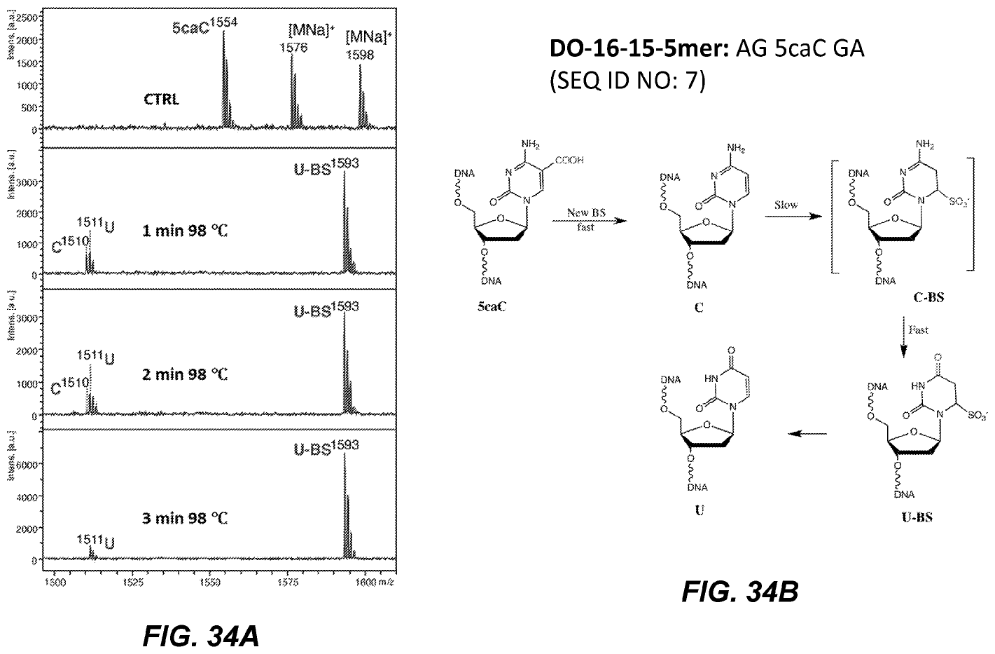

DNA oligonucleotides AGXGA (X=5hmC, 5fC or 5caC) (SEQ ID NOs: 5, 6, 7) were incubated with the BS recipe A7 at 98° C. for different amounts of time and MALDI-TOF MS was used to monitor the reactions. It was found that the reaction of 5hmC with BS was the most efficient, with 5hmC converted to the corresponding cytosine methylene sulfonate (CMS) within only 1 min (FIG. 32A), while the reaction with 5fC was the slowest, with 5fC converted U-BS adduct within 30 min. After desulphonation under basic conditions, U-BS was quantitatively converted to U and therefore, 5fC would be converted to U in BS sequencing (FIG. 33A). 5caC also reacted with BS reagent very quickly; the reaction was completed within 3 min and quantitively converted 5caC to U-BS adduct (FIG. 34A). Therefore, both 5fC and 5caC would be read as U in BS-seq, while 5mC and 5hmC remain to be read as C.

A known drawback for BS sequencing is that it cannot distinguish 5mC from 5hmC since both of them are read as C after BS treatment, although the chemistry is different since 5mC does not react with BS at all while 5hmC is converted to CMS upon BS treatment. Recently, ACE-seq²⁸ was reported to sequence 5hmC by taking advantage of the high deamination reactivity of APOBEC3A on C and 5mC, although 5hmC could be partially deaminated as well.

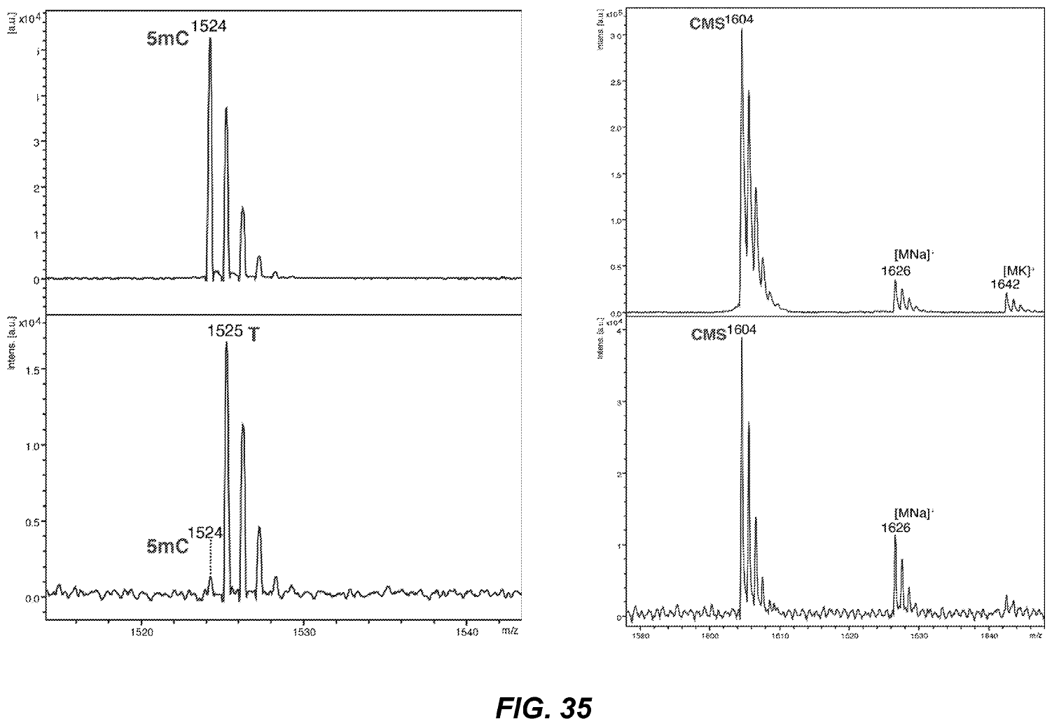

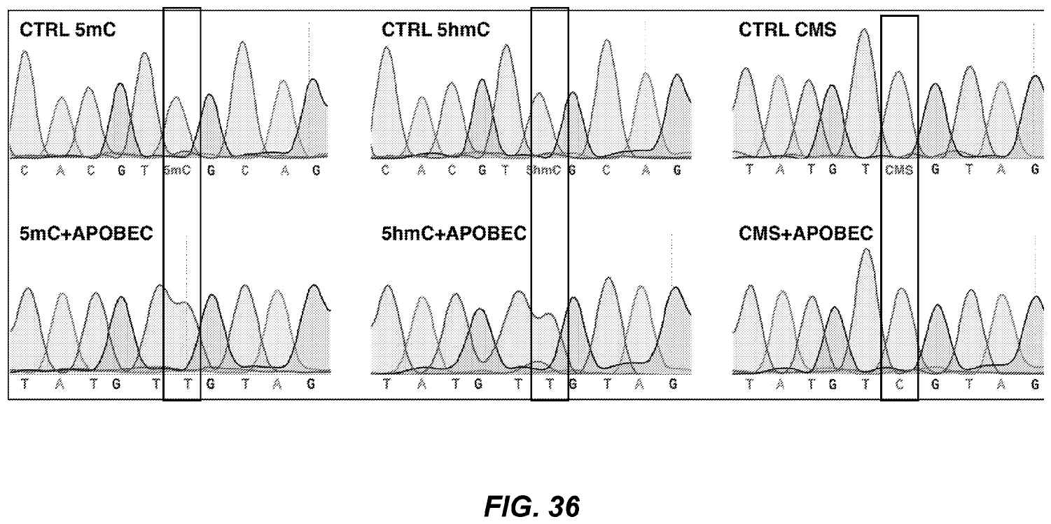

Since the disclosed BS conditions convert 5hmC spontaneously to CMS quantitatively, it was hypothesized that CMS would not be deaminated by APOBEC3A treatment. To test the hypothesis, a DNA 5mer oligo containing a 5hmC was treated with BS recipe A7 to convert 5hmC to CMS, and then 5mC-containing or CMS-containing probes were treated side-by-side with APOBEC3A. Maldi-TOF MS showed 5mC was efficiently converted to T, consistent with the literature. However, no reaction was observed for CMS (FIG. 35). 82mer DNA oligos containing a 5mC, 5hmC or CMS were used to test this further. Without APOBEC3A treatment, all of them were read as C in Sanger sequencing. However, after APOBEC3A treatment, 5mC was read T quantitatively, 5hmC was partially read as T while CMS was read as C quantitatively (FIG. 36), further confirming that one may use this new property of CMS to distinguish 5mC from 5hmC.

A new approach to sequence 5mC and 5hmC and a way to distinguish them is provided herein. As shown in FIG. 37, one can treat biological DNA with the new BS conditions and then split the sample into two parts. One part without further APOBEC3A treatment will provide 5mC+5hmC sites, while the other part with further APOBEC3A treatment will convert 5mC to T but keep CMS intact, and thus only original 5hmC sites will be read as C. Then subtraction of the two sets of data will give 5mC sites only.

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Chen, X. et al. 5-methylcytosine promotes pathogenesis of bladder cancer through stabilizing mRNAs. Nat. Cell Biol. 21, 978-990 (2019).
2. He, Y. et al. Role of m⁵C-related regulatory genes in the diagnosis and prognosis of hepatocellular carcinoma. Am. J. Transl. Res. 12, 912-922 (2020).
3. Cheray, M. et al. Cytosine methylation of mature microRNAs inhibits their functions and is associated with poor prognosis in glioblastoma multiforme. Mol. Cancer 19, 36 (2020).
4. Cheng, J. X. et al. RNA cytosine methylation and methyltransferases mediate chromatin organization and 5-azacytidine response and resistance in leukaemia. Nat. Commun. 9, 1163 (2018).
5. Edelheit, S., Schwartz, S., Mumbach, M. R., Wurtzel, O. & Sorek, R. Transcriptome—wide mapping of 5-methylcytidine RNA modifications in bacteria, archaea, and yeast reveals m⁵C within archaeal mRNAs. PLoS Genet. 9, e1003602 (2013).
6. Khoddami, V. & Cairns, B. R. Identification of direct targets and modified bases of RNA cytosine methyltransferases. Nat. Biotechnol. 31, 458-464 (2013).
7. Hussain, S., Aleksic, J., Blanco, S., Dietmann, S. & Frye, M. Characterizing 5-methylcytosine in the mammalian epitranscriptome. Genome Biol. 14, 215 (2013).
8. Schaefer, M., Pollex, T., Hanna, K. & Lyko, F. RNA cytosine methylation analysis by bisulfite sequencing. Nucleic Acids Res. 37, e12 (2009).
9. Cui, X. et al. 5-Methylcytosine RNA Methylation in Arabidopsis Thaliana. Mol. Plant 10, 1387-1399 (2017).
10. Yang, X. et al. 5-methylcytosine promotes mRNA export—NSUN2 as the methyltransferase and ALYREF as an m⁵C reader. Cell Res. 27, 606-625 (2017).
11. Janin, M. et al. Epigenetic loss of RNA-methyltransferase NSUN5 in glioma targets ribosomes to drive a stress adaptive translational program. Acta Neuropathol. (Berl.) 138, 1053-1074 (2019).
12. Blanco, S. et al. Aberrant methylation of tRNAs links cellular stress to neuro-developmental disorders. EMBO J. 33, 2020-2039 (2014).
13. Squires, J. E. et al. Widespread occurrence of 5-methylcytosine in human coding and non-coding RNA. Nucleic Acids Res. 40, 5023-5033 (2012).
14. Legrand, C. et al. Statistically robust methylation calling for whole-transcriptome bisulfite sequencing reveals distinct methylation patterns for mouse RNAs. Genome Res. 27, 1589-1596 (2017).
15. Huang, T., Chen, W., Liu, J., Gu, N. & Zhang, R. Genome-wide identification of mRNA 5-methylcytosine in mammals. Nat. Struct. Mol. Biol. 26, 380-388 (2019).
16. Sono, M., Wataya, Y. & Hayatsu, H. Role of bisulfite in the deamination and the hydrogen isotope exchange of cytidylic acid. J. Am. Chem. Soc. 95, 4745-4749 (1973).
17. Shapiro, R., DiFate, V. & Welcher, M. Deamination of cytosine derivatives by bisulfite. Mechanism of the reaction. J. Am. Chem. Soc. 96, 906-912 (1974).
18. Hayatsu, H., Negishi, K. & Shiraishi, M. DNA methylation analysis: speedup of bisulfite-mediated deamination of cytosine in the genomic sequencing procedure. Proc. Jpn. Acad. Ser. B Phys. Biol. Sci. 80, 189 (2004).
19. Tanaka, K. & Okamoto, A. Degradation of DNA by bisulfite treatment. Bioorg. Med. Chem. Lett. 17, 1912-1915 (2007).
20. Shiraishi, M. & Hayatsu, H. High-speed conversion of cytosine to uracil in bisulfite genomic sequencing analysis of DNA methylation. DNA Res. Int. J. Rapid Publ. Rep. Genes Genomes 11, 409-415 (2004).
21. Zhang, Z. et al. Systematic calibration of epitranscriptomic maps using a synthetic modification-free RNA library. Nat. Methods 18, 1213-1222 (2021).
22. Hayatsu, H. Discovery of bisulfite-mediated cytosine conversion to uracil, the key reaction for DNA methylation analysis—a personal account. Proc. Jpn. Acad. Ser. B Phys. Biol. Sci. 84, 321-330 (2008).
23. Hayatsu, H. Bisulfite Modification of Cytosine and 5-Methylcytosine as used in Epigenetic Studies. Genes Environ. 28, 1-8 (2006).
24. Hayatsu, H. The bisulfite genomic sequencing used in the analysis of epigenetic states, a technique in the emerging environmental genotoxicology research. Mutat. Res. 659, 77-82 (2008).
25. Genereux, D. P., Johnson, W. C., Burden, A. F., Stöger, R. & Laird, C. D. Errors in the bisulfite conversion of DNA: modulating inappropriate- and failed-conversion frequencies. Nucleic Acids Res. 36, e150 (2008).
26. Yi, S., Long, F., Cheng, J. & Huang, D. An optimized rapid bisulfite conversion method with high recovery of cell-free DNA. BMC Mol. Biol. 18, 24 (2017).
27. Olova, N. et al. Comparison of whole-genome bisulfite sequencing library preparation strategies identifies sources of biases affecting DNA methylation data. Genome Biol. 19, 33 (2018).
28. Schutsky, E. K. et al. Nondestructive, base-resolution sequencing of 5-hydroxymethylcytosine using a DNA deaminase. Nat. Biotechnol. (2018) doi:10.1038/nbt.4204.

What is claimed is:

1. A method for DNA processing, the method comprising:
(a) incubating a solution comprising a DNA molecule and ammonium bisulfite at a temperature of at least 95° C. for at most 12 minutes, wherein the solution does not comprise added sodium bisulfite; and
(b) subjecting the DNA molecule to alkaline conditions.

2. The method of claim 1, wherein the solution does not comprise added ammonium sulfite.

3. The method of claim 1 or 2, wherein the solution does not comprise ammonium sulfite at levels greater than about 1/10^{th} the levels of ammonium bisulfite.

4. The method of claim 1 or 2, wherein the solution does not comprise sodium bisulfite at levels greater than about 1/10^{th} the levels of ammonium bisulfite.

5. The method of claim 1 or 2, wherein the solution is at a bisulfite concentration between 6.5 M and 10 M.

6. The method of claim 1 or 2, wherein the solution is at a bisulfite concentration between 8 M and 10 M.

7. The method of claim 1 or 2, wherein the solution is at a bisulfite concentration between 9 M and 10 M.

8. The method of claim 1 or 2, wherein the solution is at a bisulfite concentration of about 9.5 M.

9. The method of claim 1 or 2, wherein the solution comprises between 50% and 70% ammonium bisulfite by weight.

10. The method of claim 9, wherein the solution comprises between 60% and 70% ammonium bisulfite by weight.

11. The method of claim 10, wherein the solution comprises between 65% and 68% ammonium bisulfite by weight.

12. The method of claim 11, wherein the solution comprises about 66.7% ammonium bisulfite by weight.

13. The method of claim 1 or 2, wherein the solution has a pH between 4.8-5.4.

14. The method claim 13, wherein the solution has a pH of about 5.1.

15. The method of claim 1 or 2, wherein (a) comprises incubating the solution at a temperature of about 98° C.

16. The method of claim 15, wherein (a) comprises incubating the solution for at most 10 minutes.

17. The method of claim 16, wherein (a) comprises incubating the solution for at most 8 minutes.

18. The method of claim 1 or 2, wherein the DNA molecule comprises N4-methylcytosine (4mC), and wherein greater than 50% of the 4mC is deaminated after the incubation.

19. The method of claim 18, wherein greater than 75% of the 4mC is deaminated after the incubation.

20. The method of claim 19, wherein substantially all of the 4mC is deaminated after the incubation.

21. A method for DNA processing, the method comprising:
(a) generating a solution comprising a DNA molecule and ammonium bisulfite, wherein the solution does not comprise added sodium bisulfite;
(b) incubating the solution at a temperature of at least 95° C.; and
(c) removing the DNA molecule from the solution at most 12 minutes after (a).

22. The method of claim 21, wherein the solution does not comprise added ammonium sulfite.

23. The method of claim 21, wherein the solution does not comprise ammonium sulfite at levels greater than about 1/10^{th} the levels of ammonium bisulfite.

24. The method of claim 21 or 22, wherein the solution does not comprise sodium bisulfite at levels greater than about 1/10^{th} the levels of ammonium bisulfite.

25. The method of claim 21 or 22, wherein the solution is at a bisulfite concentration between 6.5 M and 10 M.

26. The method of claim 21 or 22, wherein the solution is at a bisulfite concentration between 8 M and 10 M.

27. The method of claim 21 or 22, wherein the solution is at a bisulfite concentration between 9 M and 10 M.

28. The method of claim 21 or 22, wherein the solution is at a bisulfite concentration of about 9.5 M.

29. The method of claim 21 or 22, wherein the solution comprises between 50% and 70% ammonium bisulfite by weight.

30. The method of claim 29, wherein the solution comprises between 60% and 70% ammonium bisulfite by weight.

31. The method of claim 30, wherein the solution comprises between 65% and 68% ammonium bisulfite by weight.

32. The method of claim 31, wherein the solution comprises about 66.7% ammonium bisulfite by weight.

33. The method of claim 21 or 22, wherein the solution has a pH between 4.8-5.4.

34. The method of claim 33, wherein the solution has a pH of about 5.1.

35. The method of claim 21 or 22, wherein (b) comprises incubating the solution at a temperature of about 98° C.

36. The method of claim 21 or 22, wherein (c) comprises removing the DNA molecule from the solution at most 10 minutes after (a).

37. The method of claim 36, wherein (c) comprises removing the DNA molecule from the solution at most 8 minutes after (a).

38. The method of claim 21 or 22, wherein (a) comprises mixing a 70% ammonium bisulfite solution and a 50% bisulfite solution.

39. The method of claim 21 or 22, wherein the DNA molecule comprises 4mC, and wherein greater than 50% of the 4mC is deaminated after the incubation.

40. The method of claim 39, wherein greater than 75% of the 4mC is deaminated after the incubation.

41. The method of claim 40, wherein substantially all of the 4mC is deaminated after the incubation.

42. A method for processing a nucleic acid sample, the method comprising incubating a solution comprising DNA molecules and ammonium bisulfite at a temperature of at least 95° C. for at most 12 minutes, wherein the solution does not comprise added sodium bisulfite, wherein the DNA molecules each comprise one or more cytosine residues, and wherein, after incubating the solution, greater than 99% of the DNA molecules comprise no cytosine residue.

43. The method of claim 42, wherein the solution does not comprise sodium bisulfite at levels greater than about 1/10^{th} the levels of ammonium bisulfite.

44. The method of claim 42, further comprising subjecting the plurality of DNA molecules to alkaline conditions.

45. The method of claim 42 or 44, wherein the solution comprises between 50% and 70% ammonium bisulfite by weight.

46. The method of claim 42 or 44, wherein the solution comprises between 60% and 70% ammonium bisulfite by weight.

47. The method of claim 42 or 44, wherein the solution comprises between 65% and 68% ammonium bisulfite by weight.

48. The method of claim 42 or 44, wherein the solution comprises about 66.7% ammonium bisulfite by weight.

49. The method of claim 42 or 44, wherein the solution does not comprise added ammonium sulfite.

50. The method of claim 42 or 44, wherein the solution does not comprise ammonium sulfite at levels greater than about 1/10^{th} the levels of ammonium bisulfite.

51. The method of claim 42 or 44, wherein the solution is at a bisulfite concentration between 6.5 M and 10 M.

52. The method of claim 51, wherein the solution is at a bisulfite concentration between 8 M and 10 M.

53. The method of claim 52, wherein the solution is at a bisulfite concentration between 9 M and 10 M.

54. The method of claim 53, wherein the solution is at a bisulfite concentration of about 9.5 M.

55. The method of claim 42 or 44, wherein the solution has a pH between 4.8-5.4.

56. The method of claim 42 or 44, wherein the DNA molecule comprises 4mC, and greater than 50% of the 4mC is deaminated after the incubation.

57. The method of claim 56, wherein greater than 75% of the 4mC is deaminated after the incubation.

58. The method of claim 57, wherein substantially all of the 4mC is deaminated after the incubation.

59. A DNA processing kit comprising:
(a) a solution comprising ammonium bisulfite having a bisulfite concentration between 6.5 M and 10 M, wherein the solution does not comprise sodium bisulfite; and
(b) instructions for processing a DNA sample.

60. The kit of claim 59, wherein the solution does not comprise sodium bisulfite at levels greater than about 1/10^{th} the levels of ammonium bisulfite.

61. The kit of claim 59, wherein the solution is at a bisulfite concentration between 8 M and 10 M.

62. The kit of claim 59, wherein the solution is at a bisulfite concentration between 9 M and 10 M.

63. The kit of claim 59, wherein the solution is at a bisulfite concentration of about 9.5 M.

64. The kit of claim 59, wherein the solution comprises between 50% and 70% ammonium bisulfite by weight.

65. The kit of claim 64, wherein the solution comprises between 60% and 70% ammonium bisulfite by weight.

66. The kit of claim 65, wherein the solution comprises between 65% and 68% ammonium bisulfite by weight.

67. The kit of claim 66, wherein the solution comprises about 66.7% ammonium bisulfite by weight.

68. The kit of claim 59 or 64, wherein the solution has a pH between 4.8-5.4.

69. The kit of claim 68, wherein the solution has a pH of about 5.1.

70. The kit of claim 59 or 64, wherein the instructions comprise instructions for incubating the DNA sample with the solution at a temperature of at least 95° C. for at most 12 minutes.

71. The kit of claim 59 or 64, wherein the instructions comprise instructions for incubating the DNA sample with the solution at a temperature of about 98° C.

72. The kit of claim 59 or 64, wherein the instructions comprise instructions for incubating the DNA sample with the solution for at most 10 minutes.

73. The kit of claim 70, wherein the instructions comprise instructions for incubating the DNA sample with the solution for at most 8 minutes.

74. The kit of claim 59 or 64, wherein the solution does not comprise added ammonium sulfite.

75. The kit of claim 59 or 64, wherein the solution does not comprise ammonium sulfite at levels greater than about 1/10^{th} the levels of ammonium bisulfite.

76. The kit of claim 59 or 64, further comprising an alkaline solution.

77. The kit of claim 59 or 64, further comprising one or more buffer solutions.

78. A method for RNA processing, the method comprising:
(a) incubating a solution comprising an RNA molecule, ammonium sulfite, and ammonium bisulfite at a temperature of at least 95° C. for at most 12 minutes, wherein the solution does not comprise added sodium bisulfite; and
(b) subjecting the RNA molecule to alkaline conditions.

79. The method of claim 78, wherein the solution does not comprise sodium bisulfite at levels greater than about 1/10^{th} the levels of ammonium sulfite.

80. The method of claim 78, wherein the solution does not comprise sodium bisulfite at levels greater than about 1/10^{th} the levels of ammonium bisulfite.

81. The method of claim 78, wherein the solution is at a bisulfite concentration between 6.5 M and 10 M.

82. The method of claim 78, wherein the solution is at a bisulfite concentration between 6.5 M and 7.5M.

83. The method of claim 78, wherein the solution is at a bisulfite concentration of about 7.0 M.

84. The method of claim 78 or 81, wherein the solution has a pH between 4.8-5.4.

85. The method of claim 78 or 81, wherein the solution comprises between 5% and 15% ammonium sulfite by weight.

86. The method of claim 85, wherein the solution comprises between 8% and 12% ammonium sulfite by weight.

87. The method of claim 86, wherein the solution comprises about 10% ammonium sulfite by weight.

88. The method of claim 78 or 81, wherein (a) comprises incubating the solution at a temperature of about 98° C.

89. The method of claim 78 or 81, wherein (a) comprises incubating the solution for at most 10 minutes.

90. The method of claim 89, wherein (a) comprises incubating the solution for at most 8 minutes.

91. A method for RNA processing, the method comprising:
(a) generating a solution comprising an RNA molecule, ammonium sulfite, and ammonium bisulfite, wherein the solution does not comprise added sodium bisulfite;
(b) incubating the solution at a temperature of at least 95° C.; and
(c) removing the RNA molecule from the solution at most 12 minutes after (a).

92. The method of claim 91, wherein the solution does not comprise sodium bisulfite at levels greater than about 1/10^{th} the levels of ammonium sulfite.

93. The method of claim 91, wherein the solution does not comprise sodium bisulfite at levels greater than about 1/10^{th} the levels of ammonium bisulfite.

94. The method of claim 91, wherein the solution has a bisulfite concentration between 6.5 M-10 M.

95. The method of claim 91, wherein the solution has a bisulfite concentration between 6.5 M and 7.5M.

96. The method of claim 91, wherein the solution has a bisulfite concentration of about 7.0 M.

97. The method of claim 91 or 94, wherein the solution has a pH between 4.8-5.4.

98. The method of claim 97, wherein the solution has a pH of about 5.1.

99. The method of claim 91 or 94, wherein the solution comprises between 5% and 15% ammonium sulfite by weight.

100. The method of claim 99, wherein the solution comprises between 8% and 12% ammonium sulfite by weight.

101. The method of claim 100, wherein the solution comprises about 10% ammonium sulfite by weight.

102. The method of claim 91 or 94, wherein (b) comprises incubating the solution at a temperature of about 98° C.

103. The method of claim 91 or 94, wherein (c) comprises removing the RNA molecule from the solution at most 10 minutes after (a).

104. The method of claim 103, wherein (c) comprises removing the RNA molecule from the solution at most 8 minutes after (a).

105. A method for processing a nucleic acid sample, the method comprising incubating a solution comprising RNA molecules, ammonium sulfite, and ammonium bisulfite at a temperature of at least 95° C. for at most 12 minutes, wherein the solution does not comprise added sodium bisulfite, wherein the RNA molecules each comprise one or more cytosine residues, wherein, after incubating the solution, greater than 99% of the RNA molecules comprise no cytosine residue.

106. The method of claim 105, wherein the solution does not comprise sodium bisulfite at levels greater than about 1/10^{th} the levels of ammonium sulfite.

107. The method of claim 105, wherein the solution does not comprise sodium bisulfite at levels greater than about 1/10^{th} the levels of ammonium bisulfite.

108. The method of claim 105, wherein the solution has a pH between 4.8-5.4.

109. The method of claim 108, wherein the solution has a pH of about 5.1.

110. The method of claim 105 or 108, wherein the solution comprises between 5% and 15% ammonium sulfite by weight.

111. The method of claim 105 or 108, wherein the solution comprises between 8% and 12% ammonium sulfite by weight.

112. The method of claim 111, wherein the solution comprises about 10% ammonium sulfite by weight.

113. The method of claim 105 or 108, wherein (a) comprises incubating the solution at a temperature of about 98° C.

114. The method of claim 105 or 108, wherein (a) comprises incubating the solution for at most 10 minutes.

115. The method of claim 114, wherein (a) comprises incubating the solution for at most 8 minutes.

116. The method of claim 105 or 108, wherein the solution has a bisulfite concentration between 6.5 M-10 M.

117. The method of claim 116, wherein the solution has a bisulfite concentration between 6.5 M and 7.5M

118. The method of claim 117, wherein the solution has a bisulfite concentration of about 7.0 M.

119. The method of claim 105 or 108, further comprising subjecting the plurality of RNA molecules to alkaline conditions.

120. An RNA processing kit comprising:
(a) a solution comprising ammonium sulfite and ammonium bisulfite at a bisulfite concentration between 6.5 M-8 M, wherein the solution does not comprise sodium bisulfite;
(b) instructions for processing an RNA sample.

121. The kit of claim 120, wherein the solution does not comprise sodium bisulfite at levels greater than about 1/10^{th} the levels of ammonium sulfite.

122. The kit of claim 120, wherein the solution does not comprise sodium bisulfite at levels greater than about 1/10^{th} the levels of ammonium bisulfite.

123. The kit of claim 120, wherein the solution is at a bisulfite concentration of about 7.0 M.

124. The kit of claim 120 or 123, wherein the solution has a pH between 4.8-5.4.

125. The kit of claim 124, wherein the solution has a pH of about 5.1.

126. The kit of claim 120 or 123, wherein the solution comprises between 5% and 15% ammonium sulfite by weight.

127. The kit of claim 126, wherein the solution comprises between 8% and 12% ammonium sulfite by weight.

128. The kit of claim 127, wherein the solution comprises about 10% ammonium sulfite by weight.

129. The kit of claim 120 or 123, wherein the instructions comprise instructions for incubating the RNA sample with the solution at a temperature of at least 95° C. for at most 12 minutes.

130. The kit of claim 120 or 123, wherein the instructions comprise instructions for incubating the RNA sample with the solution at a temperature of about 98° C.

131. The kit of claim 120 or 123, wherein the instructions comprise instructions for incubating the RNA sample with the solution for at most 10 minutes.

132. A method for 5-hydroxymethylcytosine analysis, the method comprising:
(a) incubating a first solution comprising a first DNA molecule and ammonium bisulfite at a temperature of at least 95° C. for at most 12 minutes;
(b) incubating a second solution comprising a second DNA molecule and ammonium bisulfite at a temperature of at least 95° C. for at most 12 minutes;
(c) subjecting the first DNA molecule to alkaline conditions;
(d) subjecting the second DNA molecule to alkaline conditions;
(e) treating the second DNA molecule with an APOBEC deaminase enzyme; and
(f) sequencing the first DNA molecule and the second DNA molecule.

133. The method of claim 132, wherein the first solution does not comprise added sodium bisulfite.

134. The method of claim 132, wherein the first solution does not comprise sodium bisulfite at levels greater than about 1/10^{th} the levels of ammonium bisulfite.

135. The method of claim 132 or 133, wherein the second solution does not comprise added sodium bisulfite.

136. The method of claim 132, wherein the second solution does not comprise sodium bisulfite at levels greater than about 1/10^{th} the levels of ammonium bisulfite.

137. The method of claim 132, wherein the first solution and the second solution are the same solution.

138. The method of claim 132, wherein the first solution and the second solution are different solutions.

139. The method of claim 132 or 133, wherein (a) and (b) are performed simultaneously.

140. The method of claim 132 or 133, wherein (c) and (d) are performed simultaneously.

141. The method of claim 132 or 133, wherein the first DNA molecule and the second DNA molecule have the same nucleotide sequence.

142. The method of claim 132 or 133, wherein the APOBEC deaminase enzyme is APOBEC3A.


SEQUENCE LISTING (17 sequences)

SEQ ID NO: 1-7 - skipped (7 entries)

SEQ ID NO: 8 - DNA, 82 bp, synthetic construct
  source 1..82: mol_type = other DNA
  modified_base 56: mod_base = m5c
gtgagtggagttgagaggtgtggtcttccgatctagatgtgtagtgccatcacgtcgcaggttgaggggtgtagtgaggggt

SEQ ID NO: 9-12 - skipped (4 entries)

SEQ ID NO: 13 - DNA, 164 bp, synthetic construct
  source 1..164: mol_type = other DNA
  modified_base 77: mod_base = m5c
  modified_base 127: mod_base = m5c
gtgagtggagttgagaggtgtggtacggtgactcaggtttgtgctcttccgatctagatgtgtagtgccatccgatcgcatatgcgagtcacgtacatgctactgtcagtactgatggacctttctcgcagtggcgactatggttgaggggtgtagtgaggggt

SEQ ID NO: 14 - DNA, 164 bp, synthetic construct
  source 1..164: mol_type = other DNA
  modified_base 37: mod_base = m5c
  modified_base 72: mod_base = m5c
acccctcactacacccctcaaccatagtcgccactgcgagaaaggtccatcagtactgacagtagcatgtacgtgactcgcatatgcgatcggatggcactacacatctagatcggaagagcacaaacctgagtcaccgtaccacacctctcaactccactcac

SEQ ID NO: 15 - DNA, 48502 bp, Lambdavirus lambda
  source 1..48502: mol_type = genomic DNA
gggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcaacaaaaagcagctggctgacattttcggtgcgagtatccgtaccattcagaactggcaggaacagggaatgcccgttctgcgaggcggtggcaagggtaatgaggtgctttatgactctgccgccgtcataaaatggtatgccgaaagggatgctgaaattgagaacgaaaagctgcgccgggaggttgaagaactgcggcaggccagcgaggcagatctccagccaggaactattgagtacgaacgccatcgacttacgcgtgcgcaggccgacgcacaggaactgaagaatgccagagactccgctgaagtggtggaaaccgcattctgtactttcgtgctgtcgcggatcgcaggtgaaattgccagtattctcgacgggctccccctgtcggtgcagcggcgttttccggaactggaaaaccgacatgttgatttcctgaaacgggatatcatcaaagccatgaacaaagcagccgcgctggatgaactgataccggggttgctgagtgaatatatcgaacagtcaggttaacaggctgcggcattttgtccgcgccgggcttcgctcactgttcaggccggagccacagaccgccgttgaatgggcggatgctaattactatctcccgaaagaatccgcataccaggaagggcgctgggaaacactgccctttcagcgggccatcatgaatgcgatgggcagcgactacatccgtgaggtgaatgtggtgaagtctgcccgtgtcggttattccaaaatgctgctgggtgtttatgcctactttatagagcataagcagcgcaacacccttatctggttgccgacggatggtgatgccgagaactttatgaaaacccacgttgagccgactattcgtgatattccgtcgctgctggcgctggccccgtggtatggcaaaaagcaccgggataacacgctcaccatgaagcgtttcactaatgggcgtggcttctggtgcctgggcggtaaagcggcaaaaaactaccgtgaaaagtcggtggatgtggcgggttatgatgaacttgctgcttttgatgatgatattgaacaggaaggctctccgacgttcctgggtgacaagcgtattgaaggctcggtctggccaaagtccatccgtggctccacgccaaaagtgagaggcacctgtcagattgagcgtgcagccagtgaatccccgcattttatgcgttttcatgttgcctgcccgcattgcggggaggagcagtatcttaaatttggcgacaaagagacgccgtttggcctcaaatggacgccggatgacccctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggcgggattgacccgaccattgtgtatgaacgctcgaaaaaacatgggctgttccgggtgatccccattaaaggggcatccgtctacggaaagccggtggccagcatgccacgtaagcgaaacaaaaacggggtttaccttaccgaaatcggtacggataccgcgaaagagcagatttataaccgcttcacactgacgccggaaggggatgaaccgcttcccggtgccgttcacttcccgaataacccggatatttttgatctgaccgaagcgcagcagctgactgctgaagagcaggtcgaaaaatgggtggatggcaggaaaaaaatactgtgggacagcaaaaagcgacgcaatgaggcactcgactgcttcgtttatgcgctggcggcgctgcgcatcagtatttcccgctggcagctggatctcagtgcgctgctggcgagcctgcaggaagaggatggtgcagcaaccaacaagaaaacactggcagattacgcccgtgccttatccggagaggatgaatgacgcgacaggaagaacttgccgctgcccgtgcggcactgcatgacctgatgacaggtaaacgggtggcaacagtacagaaagacggacgaagggtggagtttacggccacttccgtgtctgacctgaaaaaatatattgcagagctggaagtgcagaccggcatgacacagcgacgcaggggacctgcaggattttatgtatgaaaacgcccaccattcccacccttctggggccggacggcatgacatcgctgcgcgaatatgccggttatcacggcggtggcagcggatttggagggcagttgcggtcgtggaacccaccgagtgaaagtgtggatgcagccctgttgcccaactttacccgtggcaatgcccgcgcagacgatctggtacgcaataacggctatgccgccaacgccatccagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcctcgttcattcacgtttttgaacccgtggaggacgggcagactcgcggtgcaaatgtgttttacagcgtgatggagcagatgaagatgctcgacacgctgcagaacacgcagctgcagagcgccattgtgaaggcgatgtatgccgccaccattgagagtgagctggatacgcagtcagcgatggattttattctgggcgcgaacagtcaggagcagcgggaaaggctgaccggctggattggtgaaattgccgcgtattacgccgcagcgccggtccggctgggaggcgcaaaagtaccgcacctgatgccgggtgactcactgaacctgcagacggctcaggatacggataacggctactccgtgtttgagcagtcactgctgcggtatatcgctgccgggctgggtgtctcgtatgagcagctttcccggaattacgcccagatgagctactccacggcacgggccagtgcgaacgagtcgtgggcgtactttatggggcggcgaaaattcgtcgcatcccgtcaggcgagccagatgtttctgtgctggctggaagaggccatcgttcgccgcgtggtgacgttaccttcaaaagcgcgcttcagttttcaggaagcccgcagtgcctgggggaactgcgactggataggctccggtcgtatggccatcgatggtctgaaagaagttcaggaagcggtgatgctgatagaagccggactgagtacctacgagaaagagtgcgcaaaacgcggtgacgactatcaggaaatttttgcccagcaggtccgtgaaacgatggagcgccgtgcagccggtcttaaaccgcccgcctgggcggctgcagcatttgaatccgggctgcgacaatcaacagaggaggagaagagtgacagcagagctgcgtaatctcccgcatattgccagcatggcctttaatgagccgctgatgcttgaacccgcctatgcgcgggttttcttttgtgcgcttgcaggccagcttgggatcagcagcctgacggatgcggtgtccggcgacagcctgactgcccaggaggcactcgcgacgctggcattatccggtgatgatgacggaccacgacaggcccgcagttatcaggtcatgaacggcatcgccgtgctgccggtgtccggcacgctggtcagccggacgcgggcgctgcagccgtactcggggatgaccggttacaacggcattatcgcccgtctgcaacaggctgccagcgatccgatggtggacggcattctgctcgatatggacacgcccggcgggatggtggcgggggcatttgactgcgctgacatcatcgcccgtgtgcgtgacataaaaccggtatgggcgcttgccaacgacatgaactgcagtgcaggtcagttgcttgccagtgccgcctcccggcgtctggtcacgcagaccgcccggacaggctccatcggcgtcatgatggctcacagtaattacggtgctgcgctggagaaacagggtgtggaaatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtgaatccgcagatgaccctgcgtcgcctgccggatgaagatccgcagaatctggcggacccggcttaccgccgccgtcgcatcatcatgcagaacatgcgtgacgaagagctggccattgctcaggtcgaagagatgcaggcagtttctgccgtgcttaagggcaaatacaccatgaccggtgaagccttcgatccggttgaggtggatatgggccgcagtgaggagaataacatcacgcagtccggcggcacggagtggagcaagcgtgacaagtccacgtatgacccgaccgacgatatcgaagcctacgcgctgaacgccagcggtgtggtgaatatcatcgtgttcgatccgaaaggctgggcgctgttccgttccttcaaagccgtcaaggagaagctggatacccgtcgtggctctaattccgagctggagacagcggtgaaagacctgggcaaagcggtgtcctataaggggatgtatggcgatgtggccatcgtcgtgtattccggacagtacgtggaaaacggcgtcaaaaagaacttcctgccggacaacacgatggtgctggggaacactcaggcacgcggtctgcgcacctatggctgcattcaggatgcggacgcacagcgcgaaggcattaacgcctctgcccgttacccgaaaaactgggtgaccaccggcgatccggcgcgtgagttcaccatgattcagtcagcaccgctgatgctgctggctgaccctgatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggacggggcgtaccgcctgccgttaaccgtcgccgctgaaagggggatgtatggccataaaaggtcttgagcaggccgttgaaaacctcagccgtatcagcaaaacggcggtgcctggtgccgccgcaatggccattaaccgcgttgcttcatccgcgatatcgcagtcggcgtcacaggttgcccgtgagacaaaggtacgccggaaactggtaaaggaaagggccaggctgaaaagggccacggtcaaaaatccgcaggccagaatcaaagttaaccggggggatttgcccgtaatcaagctgggtaatgcgcgggttgtcctttcgcgccgcaggcgtcgtaaaaaggggcagcgttcatccctgaaaggtggcggcagcgtgcttgtggtgggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaatccggagagtcagcgatgttcctgaaaaccgaatcatttgaacataacggtgtgaccgtcacgctttctgaactgtcagccctgcagcgcattgagcatctcgccctgatgaaacggcaggcagaacaggcggagtcagacagcaaccggaagtttactgtggaagacgccatcagaaccggcgcgtttctggtggcgatgtccctgtggcataaccatccgcagaagacgcagatgccgtccatgaatgaagccgttaaacagattgagcaggaagtgcttaccacctggcccacggaggcaatttctcatgctgaaaacgtggtgtaccggctgtctggtatgtatgagtttgtggtgaataatgcccctgaacagacagaggacgccgggcccgcagagcctgtttctgcgggaaagtgttcgacggtgagctgagttttgccctgaaactggcgcgtgagatggggcgacccgactggcgtgccatgcttgccgggatgtcatccacggagtatgccgactggcaccgcttttacagtacccattattttcatgatgttctgctggatatgcacttttccgggctgacgtacaccgtgctcagcctgtttttcagcgatccggatatgcatccgctggatttcagtctgctgaaccggcgcgaggctgacgaagagcctgaagatgatgtgctgatgcagaaagcggcagggcttgccggaggtgtccgctttggcccggacgggaatgaagttatccccgcttccccggatgtggcggacatgacggaggatgacgtaatgctgatgacagtatcagaagggatcgcaggaggagtccggtatggctgaaccggtaggcgatctggtcgttgatttgagtctggatgcggccagatttgacgagcagatggccagagtcaggcgtcatttttctggtacggaaagtgatgcgaaaaaaacagcggcagtcgttgaacagtcgctgagccgacaggcgctggctgcacagaaagcggggatttccgtcgggcagtataaagccgccatgcgtatgctgcctgcacagttcaccgacgtggccacgcagcttgcaggcgggcaaagtccgtggctgatcctgctgcaacagggggggcaggtgaaggactccttcggcgggatgatccccatgttcagggggcttgccggtgcgatcaccctgccgatggtgggggccacctcgctggcggtggcgaccggtgcgctggcgtatgcctggtatcagggcaactcaaccctgtccgatttcaacaaaacgctggtcctttccggcaatcaggcgggactgacggcagatcgtatgctggtcctgtccagagccgggcaggcggcagggctgacgtttaaccagaccagcgagtcactcagcgcactggttaaggcgggggtaagcggtgaggctcagattgcgtccatcagccagagtgtggcgcgtttctcctctgcatccggcgtggaggtggacaaggtcgctgaagccttcgggaagctgaccacagacccgacgtcggggctgacggcgatggctcgccagttccataacgtgtcggcggagcagattgcgtatgttgctcagttgcagcgttccggcgatgaagccggggcattgcaggcggcgaacgaggccgcaacgaaagggtttgatgaccagacccgccgcctgaaagagaacatgggcacgctggagacctgggcagacaggactgcgcgggcattcaaatccatgtgggatgcggtgctggatattggtcgtcctgataccgcgcaggagatgctgattaaggcagaggctgcgtataagaaagcagacgacatctggaatctgcgcaaggatgattattttgttaacgatgaagcgcgggcgcgttactgggatgatcgtgaaaaggcccgtcttgcgcttgaagccgcccgaaagaaggctgagcagcagactcaacaggacaaaaatgcgcagcagcagagcgataccgaagcgtcacggctgaaatataccgaagaggcgcagaaggcttacgaacggctgcagacgccgctggagaaatataccgcccgtcaggaagaactgaacaaggcactgaaagacgggaaaatcctgcaggcggattacaacacgctgatggcggcggcgaaaaaggattatgaagcgacgctgaaaaagccgaaacagtccagcgtgaaggtgtctgcgggcgatcgtcaggaagacagtgctcatgctgccctgctgacgcttcaggcagaactccggacgctggagaagcatgccggagcaaatgagaaaatcagccagcagcgccgggatttgtggaaggcggagagtcagttcgcggtactggaggaggcggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaacgggcaaaacgggccgccattgatgcgaaaagccgggggctgactgaccggcaggcagaacgggaagccacggaacagcgcctgaaggaacagtatggcgataatccgctggcgctgaataacgtcatgtcagagcagaaaaagacctgggcggctgaagaccagcttcgcgggaactggatggcaggcctgaagtccggctggagtgagtgggaagagagcgccacggacagtatgtcgcaggtaaaaagtgcagccacgcagacctttgatggtattgcacagaatatggcggcgatgctgaccggcagtgagcagaactggcgcagcttcacccgttccgtgctgtccatgatgacagaaattctgcttaagcaggcaatggtggggattgtcgggagtatcggcagcgccattggcggggctgttggtggcggcgcatccgcgtcaggcggtacagccattcaggccgctgcggcgaaattccattttgcaaccggaggatttacgggaaccggcggcaaatatgagccagcggggattgttcaccgtggtgagtttgtcttcacgaaggaggcaaccagccggattggcgtggggaatctttaccggctgatgcgcggctatgccaccggcggttatgtcggtacaccgggcagcatggcagacagccggtcgcaggcgtccgggacgtttgagcagaataaccatgtggtgattaacaacgacggcacgaacgggcagataggtccggctgctctgaaggcggtgtatgacatggcccgcaagggtgcccgtgatgaaattcagacacagatgcgtgatggtggcctgttctccggaggtggacgatgaagaccttccgctggaaagtgaaacccggtatggatgtggcttcggtcccttctgtaagaaaggtgcgctttggtgatggctattctcagcgagcgcctgccgggctgaatgccaacctgaaaacgtacagcgtgacgctttctgtcccccgtgaggaggccacggtactggagtcgtttctggaagagcacgggggctggaaatcctttctgtggacgccgccttatgagtggcggcagataaaggtgacctgcgcaaaatggtcgtcgcgggtcagtatgctgcgtgttgagttcagcgcagagtttgaacaggtggtgaactgatgcaggatatccggcaggaaacactgaatgaatgcacccgtgcggagcagtcggccagcgtggtgctctgggaaatcgacctgacagaggtcggtggagaacgttattttttctgtaatgagcagaacgaaaaaggtgagccggtcacctggcaggggcgacagtatcagccgtatcccattcaggggagcggttttgaactgaatggcaaaggcaccagtacgcgccccacgctgacggtttctaacctgtacggtatggtcaccgggatggcggaagatatgcagagtctggtcggcggaacggtggtccggcgtaaggtttacgcccgttttctggatgcggtgaacttcgtcaacggaaacagttacgccgatccggagcaggaggtgatcagccgctggcgcattgagcagtgcagcgaactgagcgcggtgagtgcctcctttgtactgtccacgccgacggaaacggatggcgctgtttttccgggacgtatcatgctggccaacacctgcacctggacctatcgcggtgacgagtgcggttatagcggtccggctgtcgcggatgaatatgaccagccaacgtccgatatcacgaaggataaatgcagcaaatgcctgagcggttgtaagttccgcaataacgtcggcaactttggcggcttcctttccattaacaaactttcgcagtaaatcccatgacacagacagaatcagcgattctggcgcacgcccggcgatgtgcgccagcggagtcgtgcggcttcgtggtaagcacgccggagggggaaagatatttcccctgcgtgaatatctccggtgagccggaggctatttccgtatgtcgccggaagactggctgcaggcagaaatgcagggtgagattgtggcgctggtccacagccaccccggtggtctgccctggctgagtgaggccgaccggcggctgcaggtgcagagtgatttgccgtggtggctggtctgccgggggacgattcataagttccgctgtgtgccgcatctcaccgggcggcgctttgagcacggtgtgacggactgttacacactgttccgggatgcttatcatctggcggggattgagatgccggactttcatcgtgaggatgactggtggcgtaacggccagaatctctatctggataatctggaggcgacggggctgtatcaggtgccgttgtcagcggcacagccgggcgatgtgctgctgtgctgttttggttcatcagtgccgaatcacgccgcaatttactgcggcgacggcgagctgctgcaccatattcctgaacaactgagcaaacgagagaggtacaccgacaaatggcagcgacgcacacactccctctggcgtcaccgggcatggcgcgcatctgcctttacggggatttacaacgatttggtcgccgcatcgaccttcgtgtgaaaacgggggctgaagccatccgggcactggccacacagctcccggcgtttcgtcagaaactgagcgacggctggtatcaggtacggattgccgggcgggacgtcagcacgtccgggttaacggcgcagttacatgagactctgcctgatggcgctgtaattcatattgttcccagagtcgccggggccaagtcaggtggcgtattccagattgtcctgggggctgccgccattgccggatcattctttaccgccggagccacccttgcagcatggggggcagccattggggccggtggtatgaccggcatcctgttttctctcggtgccagtatggtgctcggtggtgtggcgcagatgctggcaccgaaagccagaactccccgtatacagacaacggataacggtaagcagaacacctatttctcctcactggataacatggttgcccagggcaatgttctgcctgttctgtacggggaaatgcgcgtggggtcacgcgtggtttctcaggagatcagcacggcagacgaaggggacggtggtcaggttgtggtgattggtcgctgatgcaaaatgttttatgtgaaaccgcctgcgggcggttttgtcatttatggagcgtgaggaatgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagtggcagtgtgaatgcgaactccgggacgctcagtaatgtgacgatagctgaaaactgtacgataaacggtacgctgagggcggaaaaaatcgtcggggacattgtaaaggcggcgagcgcggcttttccgcgccagcgtgaaagcagtgtggactggccgtcaggtacccgtactgtcaccgtgaccgatgaccatccttttgatcgccagatagtggtgcttccgctgacgtttcgcggaagtaagcgtactgtcagcggcaggacaacgtattcgatgtgttatctgaaagtactgatgaacggtgcggtgatttatgatggcgcggcgaacgaggcggtacaggtgttctcccgtattgttgacatgccagcgggtcggggaaacgtgatcctgacgttcacgcttacgtccacacggcattcggcagatattccgccgtatacgtttgccagcgatgtgcaggttatggtgattaagaaacaggcgctgggcatcagcgtggtctgagtgtgttacagaggttcgtccgggaacgggcgttttattataaaacagtgagaggtgaacgatgcgtaatgtgtgtattgccgttgctgtctttgccgcacttgcggtgacagtcactccggcccgtgcggaaggtggacatggtacgtttacggtgggctattttcaagtgaaaccgggtacattgccgtcgttgtcgggcggggataccggtgtgagtcatctgaaagggattaacgtgaagtaccgttatgagctgacggacagtgtgggggtgatggcttccctggggttcgccgcgtcgaaaaagagcagcacagtgatgaccggggaggatacgtttcactatgagagcctgcgtggacgttatgtgagcgtgatggccggaccggttttacaaatcagtaagcaggtcagtgcgtacgccatggccggagtggctcacagtcggtggtccggcagtacaatggattaccgtaagacggaaatcactcccgggtatatgaaagagacgaccactgccagggacgaaagtgcaatgcggcatacctcagtggcgtggagtgcaggtatacagattaatccggcagcgtccgtcgttgttgatattgcttatgaaggctccggcagtggcgactggcgtactgacggattcatcgttggggtcggttataaattctgattagccaggtaacacagtgttatgacagcccgccggaaccggtgggcttttttgtggggtgaatatggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgaaacgaacagaaaagcccactggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcctttccggcaggtgcgccgatcccgtggccatcagatatcgttccgtctggctacgtcctgatgcaggggcaggcgtttgacaaatcagcctacccaaaacttgctgtcgcgtatccatcgggtgtgcttcctgatatgcgaggctggacaatcaaggggaaacccgccagcggtcgtgctgtattgtctcaggaacaggatggaattaagtcgcacacccacagtgccagtgcatccggtacggatttggggacgaaaaccacatcgtcgtttgattacgggacgaaaacaacaggcagtttcgattacggcaccaaatcgacgaataacacgggggctcatgctcacagtctgagcggttcaacaggggccgcgggtgctcatgcccacacaagtggtttaaggatgaacagttctggctggagtcagtatggaacagcaaccattacaggaagtttatccacagttaaaggaaccagcacacagggtattgcttatttatcgaaaacggacagtcagggcagccacagtcactcattgtccggtacagccgtgagtgccggtgcacatgcgcatacagttggtattggtgcgcaccagcatccggttgttatcggtgctcatgcccattctttcagtattggttcacacggacacaccatcaccgttaacgctgcgggtaacgcggaaaacaccgtcaaaaacattgcatttaactatattgtgaggcttgcataatggcattcagaatgagtgaacaaccacggaccataaaaatttataatctgctggccggaactaatgaatttattggtgaaggtgacgcatatattccgcctcataccggtctgcctgcaaacagtaccgatattgcaccgccagatattccggctggctttgtggctgttttcaacagtgatgaggcatcgtggcatctcgttgaagaccatcggggtaaaaccgtctatgacgtggcttccggcgacgcgttatttatttctgaactcggtccgttaccggaaaattttacctggttatcgccgggaggggaatatcagaagtggaacggcacagcctgggtgaaggatacggaagcagaaaaactgttccggatccgggaggcggaagaaacaaaaaaaagcctgatgcaggtagccagtgagcatattgcgccgcttcaggatgctgcagatctggaaattgcaacgaaggaagaaacctcgttgctggaagcctggaagaagtatcgggtgttgctgaaccgtgttgatacatcaactgcacctgatattgagtggcctgctgtccctgttatggagtaatcgttttgtgatatgccgcagaaacgttgtatgaaataacgttctgcggttagttagtatattgtaaagctgagtattggtttatttggcgattattatcttcaggagaataatggaagttctatgactcaattgttcatagtgtttacatcaccgccaattgcttttaagactgaacgcatgaaatatggtttttcgtcatgttttgagtctgctgttgatatttctaaagtcggttttttttcttcgttttctctaactattttccatgaaatacatttttgattattatttgaatcaattccaattacctgaagtctttcatctataattggcattgtatgtattggtttattggagtagatgcttgcttttctgagccatagctctgatatccaaatgaagccataggcatttgttattttggctctgtcagctgcataacgccaaaaaatatatttatctgcttgatcttcaaatgttgtattgattaaatcaattggatggaattgtttatcataaaaaattaatgtttgaatgtgataaccgtcctttaaaaaagtcgtttctgcaagcttggctgtatagtcaactaactcttctgtcgaagtgatatttttaggcttatctaccagttttagacgctctttaatatcttcaggaattattttattgtcatattgtatcatgctaaatgacaatttgcttatggagtaatcttttaattttaaataagttattctcctggcttcatcaaataaagagtcgaatgatgttggcgaaatcacatcgtcacccattggattgtttatttgtatgccaagagagttacagcagttatacattctgccatagattatagctaaggcatgtaataattcgtaatcttttagcgtattagcgacccatcgtctttctgatttaataatagatgattcagttaaatatgaaggtaatttcttttgtgcaagtctgactaacttttttataccaatgtttaacatactttcatttgtaataaactcaatgtcattttcttcaatgtaagatgaaataagagtagcctttgcctcgctatacatttctaaatcgccttgtttttctatcgtattgcgagaatttttagcccaagccattaatggatcatttttccatttttcaataacattattgttataccaaatgtcatatcctataatctggtttttgtttttttgaataataaatgttactgttcttgcggtttggaggaattgattcaaattcaagcgaaataattcagggtcaaaatatgtatcaatgcagcatttgagcaagtgcgataaatctttaagtcttctttcccatggttttttagtcataaaactctccattttgataggttgcatgctagatgctgatatattttagaggtgataaaattaactgcttaactgtcaatgtaatacaagttgtttgatctttgcaatgattcttatcagaaaccatatagtaaattagttacacaggaaatttttaatattattattatcattcattatgtattaaaattagagttgtggcttggctctgctaacacgttgctcataggagatatggtagagccgcagacacgtcgtatgcaggaacgtgctgcggctggctggtgaacttccgatagtgcgggtgttgaatgatttccagttgctaccgattttacatattttttgcatgagagaatttgtaccacctcccaccgaccatctatgactgtacgccactgtccctaggactgctatgtgccggagcggacattacaaacgtccttctcggtgcatgccactgttgccaatgacctgcctaggaattggttagcaagttactaccggattttgtaaaaacagccctcctcatataaaaagtattcgttcacttccgataagcgtcgtaattttctatctttcatcatattctagatccctctgaaaaaatcttccgagtttgctaggcactgatacataactcttttccaataattggggaagtcattcaaatctataataggtttcagatttgcttcaataaattctgactgtagctgctgaaacgttgcggttgaactatatttccttataacttttacgaaagagtttctttgagtaatcacttcactcaagtgcttccctgcctccaaacgatacctgttagcaatatttaatagcttgaaatgatgaagagctctgtgtttgtcttcctgcctccagttcgccgggcattcaacataaaaactgatagcacccggagttccggaaacgaaatttgcatatacccattgctcacgaaaaaaaatgtccttgtcgatatagggatgaatcgcttggtgtacctcatctactgcgaaaacttgacctttctctcccatattgcagtcgcggcacgatggaactaaattaataggcatcaccgaaaattcaggataatgtgcaataggaagaaaatgatctatattttttgtctgtcctatatcaccacaaaatggacatttttcacctgatgaaacaagcatgtcatcgtaatatgttctagcgggtttgtttttatctcggagattattttcataaagcttttctaatttaacctttgtcaggttaccaactactaaggttgtaggctcaagagggtgtgtcctgtcgtaggtaaataactgacctgtcgagcttaatattctatattgttgttctttctgcaaaaaagtggggaagtgagtaatgaaattatttctaacatttatctgcatcataccttccgagcatttattaagcatttcgctataagttctcgctggaagaggtagttttttcattgtactttaccttcatctctgttcattatcatcgcttttaaaacggttcgaccttctaatcctatctgaccattataattttttagaatggtttcataagaaagctctgaatcaacggactgcgataataagtggtggtatccagaatttgtcacttcaagtaaaaacacctcacgagttaaaacacctaagttctcaccgaatgtctcaatatccggacggataatatttattgcttctcttgaccgtaggactttccacatgcaggattttggaacctcttgcagtactactggggaatgagttgcaattattgctacaccattgcgtgcatcgagtaagtcgcttaatgttcgtaaaaaagcagagagcaaaggtggatgcagatgaacctctggttcatcgaataaaactaatgacttttcgccaacgacatctactaatcttgtgatagtaaataaaacaattgcatgtccagagctcattcgaagcagatatttctggatattgtcataaaacaatttagtgaatttatcatcgtccacttgaatctgtggttcattacgtcttaactcttcatatttagaaatgaggctgatgagttccatatttgaaaagttttcatcactacttagttttttgatagcttcaagccagagttgtctttttctatctactctcatacaaccaataaatgctgaaatgaattctaagcggagatcgcctagtgattttaaactattgctggcagcattcttgagtccaatataaaagtattgtgtaccttttgctgggtcaggttgttctttaggaggagtaaaaggatcaaatgcactaaacgaaactgaaacaagcgatcgaaaatatccctttgggattcttgactcgataagtctattattttcagagaaaaaatattcattgttttctgggttggtgattgcaccaatcattccattcaaaattgttgttttaccacacccattccgcccgataaaagcatgaatgttcgtgctgggcatagaattaaccgtcacctcaaaaggtatagttaaatcactgaatccgggagcactttttctattaaatgaaaagtggaaatctgacaattctggcaaaccatttaacacacgtgcgaactgtccatgaatttctgaaagagttacccctctaagtaatgaggtgttaaggacgctttcattttcaatgtcggctaatcgatttggccatactactaaatcctgaatagctttaagaaggttatgtttaaaaccatcgcttaatttgctgagattaacatagtagtcaatgctttcacctaaggaaaaaaacatttcagggagttgactgaattttttatctattaatgaataagtgcttacttcttctttttgacctacaaaaccaattttaacatttccgatatcgcatttttcaccatgctcatcaaagacagtaagataaaacattgtaacaaaggaatagtcattccaaccatctgctcgtaggaatgccttatttttttctactgcaggaatatacccgcctctttcaataacactaaactccaacatatagtaacccttaattttattaaaataaccgcaatttatttggcggcaacacaggatctctcttttaagttactctctattacatacgttttccatctaaaaattagtagtattgaacttaacggggcatcgtattgtagttttccatatttagctttctgcttccttttggataacccactgttattcatgttgcatggtgcactgtttataccaacgatatagtctattaatgcatatatagtatcgccgaacgattagctcttcaggcttctgaagaagcgtttcaagtactaataagccgatagatagccacggacttcgtagccatttttcataagtgttaacttccgctcctcgctcataacagacattcactacagttatggcggaaaggtatgcatgctgggtgtggggaagtcgtgaaagaaaagaagtcagctgcgtcgtttgacatcactgctatcttcttactggttatgcaggtcgtagtgggtggcacacaaagctttgcactggattgcgaggctttgtgcttctctggagtgcgacaggtttgatgacaaaaaattagcgcaagaagacaaaaatcaccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttcaattttgtcccactccctgcctctgtcatcacgatactgtgatgccatggtgtccgacttatgcccgagaagatgttgagcaaacttatcgcttatctgcttctcatagagtcttgcagacaaactgcgcaactcgtgaaaggtaggcggatccccttcgaaggaaagacctgatgcttttcgtgcgcgcataaaataccttgatactgtgccggatgaaagcggttcgcgacgagtagatgcaattatggtttctccgccaagaatctctttgcatttatcaagtgtttccttcattgatattccgagagcatcaatatgcaatgctgttgggatggcaatttttacgcctgttttgctttgctcgacataaagatatccatctacgatatcagaccacttcatttcgcataaatcaccaactcgttgcccggtaacaacagccagttccattgcaagtctgagccaacatggtgatgattctgctgcttgataaattttcaggtattcgtcagccgtaagtcttgatctccttacctctgattttgctgcgcgagtggcagcgacatggtttgttgttatatggccttcagctattgcctctcggaatgcatcgctcagtgttgatctgattaacttggctgacgccgccttgccctcgtctatgtatccattgagcattgccgcaatttcttttgtggtgatgtcttcaagtggagcatcaggcagacccctccttattgctttaattttgctcatgtaatttatgagtgtcttctgcttgattcctctgctggccaggattttttcgtagcgatcaagccatgaatgtaacgtaacggaattatcactgttgattctcgctgtcagaggcttgtgtttgtgtcctgaaaataactcaatgttggcctgtatagcttcagtgattgcgattcgcctgtctctgcctaatccaaactctttacccgtccttgggtccctgtagcagtaatatccattgtttcttatataaaggttagggggtaaatcccggcgctcatgacttcgccttcttcccatttctgatcctcttcaaaaggccacctgttactggtcgatttaagtcaacctttaccgctgattcgtggaacagatactctcttccatccttaaccggaggtgggaatatcctgcattcccgaacccatcgacgaactgtttcaaggcttcttggacgtcgctggcgtgcgttccactcctgaagtgtcaagtacatcgcaaagtctccgcaattacacgcaagaaaaaaccgccatcaggcggcttggtgttctttcagttcttcaattcgaatattggttacgtctgcatgtgctatctgcgcccatatcatccagtggtcgtagcagtcgttgatgttctccgcttcgataactctgttgaatggctctccattccattctcctgtgactcggaagtgcatttatcatctccataaaacaaaacccgccgtagcgagttcagataaaataaatccccgcgagtgcgaggattgttatgtaatattgggtttaatcatctatatgttttgtacagagagggcaagtatcgtttccaccgtactcgtgataataattttgcacggtatcagtcatttctcgcacattgcagaatggggatttgtcttcattagacttataaaccttcatggaatatttgtatgccgactctatatctataccttcatctacataaacaccttcgtgatgtctgcatggagacaagacaccggatctgcacaacattgataacgcccaatctttttgctcagactctaactcattgatactcatttataaactccttgcaatgtatgtcgtttcagctaaacggtatcagcaatgtttatgtaaagaaacagtaagataatactcaacccgatgtttgagtacggtcatcatctgacactacagactctggcatcgctgtgaagacgacgcgaaattcagcattttcacaagcgttatcttttacaaaaccgatctcactctcctttgatgcgaatgccagcgtcagacatcatatgcagatactcacctgcatcctgaacccattgacctccaaccccgtaatagcgatgcgtaatgatgtcgatagttactaacgggtcttgttcgattaactgccgcagaaactcttccaggtcaccagtgcagtgcttgataacaggagtcttcccaggatggcgaacaacaagaaactggtttccgtcttcacggacttcgttgctttccagtttagcaatacgcttactcccatccgagataacaccttcgtaatactcacgctgctcgttgagttttgattttgctgtttcaagctcaacacgcagtttccctactgttagcgcaatatcctcgttctcctggtcgcggcgtttgatgtattgctggtttctttcccgttcatccagcagttccagcacaatcgatggtgttaccaattcatggaaaaggtctgcgtcaaatccccagtcgtcatgcattgcctgctctgccgcttcacgcagtgcctgagagttaatttcgctcacttcgaacctctctgtttactgataagttccagatcctcctggcaacttgcacaagtccgacaaccctgaacgaccaggcgtcttcgttcatctatcggatcgccacactcacaacaatgagtggcagatatagcctggtggttcaggcggcgcatttttattgctgtgttgcgctgtaattcttctatttctgatgctgaatcaatgatgtctgccatctttcattaatccctgaactgttggttaatacgcttgagggtgaatgcgaataataaaaaaggagcctgtagctccctgatgattttgcttttcatgttcatcgttccttaaagacgccgtttaacatgccgattgccaggcttaaatgagtcggtgtgaatcccatcagcgttaccgtttcgcggtgcttcttcagtacgctacggcaaatgtcatcgacgtttttatccggaaactgctgtctggctttttttgatttcagaattagcctgacgggcaatgctgcgaagggcgttttcctgctgaggtgtcattgaacaagtcccatgtcggcaagcataagcacacagaatatgaagcccgctgccagaaaaatgcattccgtggttgtcatacctggtttctctcatctgcttctgctttcgccaccatcatttccagcttttgtgaaagggatgcggctaacgtatgaaattcttcgtctgtttctactggtattggcacaaacctgattccaatttgagcaaggctatgtgccatctcgatactcgttcttaactcaacagaagatgctttgtgcatacagcccctcgtttattatttatctcctcagccagccgctgtgctttcagtggatttcggataacagaaaggccgggaaatacccagcctcgctttgtaacggagtagacgaaagtgattgcgcctacccggatattatcgtgaggatgcgtcatcgccattgctccccaaatacaaaaccaatttcagccagtgcctcgtccattttttcgatgaactccggcacgatctcgtcaaaactcgccatgtacttttcatcccgctcaatcacgacataatgcaggccttcacgcttcatacgcgggtcatagttggcaaagtaccaggcattttttcgcgtcacccacatgctgtactgcacctgggccatgtaagctgactttatggcctcgaaaccaccgagccggaacttcatgaaatcccgggaggtaaacgggcatttcagttcaaggccgttgccgtcactgcataaaccatcgggagagcaggcggtacgcatactttcgtcgcgatagatgatcggggattcagtaacattcacgccggaagtgaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgacagaatccatgccgacacgttcagccagcttcccagccagcgttgcgagtgcagtactcattcgttttatacctctgaatcaatatcaacctggtggtgagcaatggtttcaaccatgtaccggatgtgttctgccatgcgctcctgaaactcaacatcgtcatcaaacgcacgggtaatggattttttgctggccccgtggcgttgcaaatgatcgatgcatagcgattcaaacaggtgctggggcaggcctttttccatgtcgtctgccagttctgcctctttctcttcacgggcgagctgctggtagtgacgcgcccagctctgagcctcaagacgatcctgaatgtaataagcgttcatggctgaactcctgaaatagctgtgaaaatatcgcccgcgaaatgccgggctgattaggaaaacaggaaagggggttagtgaatgcttttgcttgatctcagtttcagtattaatatccattttttataagcgtcgacggcttcacgaaacatcttttcatcgccaataaaagtggcgatagtgaatttagtctggatagccataagtgtttgatccattctttgggactcctggctgattaagtatgtcgataaggcgtttccatccgtcacgtaatttacgggtgattcgttcaagtaaagattcggaagggcagccagcaacaggccaccctgcaatggcatattgcatggtgtgctccttatttatacataacgaaaaacgcctcgagtgaagcgttattggtatgcggtaaaaccgcactcaggcggccttgatagtcatatcatctgaatcaaatattcctgatgtatcgatatcggtaattcttattccttcgctaccatccattggaggccatccttcctgaccatttccatcattccagtcgaactcacacacaacaccatatgcatttaagtcgcttgaaattgctataagcagagcatgttgcgccagcatgattaatacagcatttaatacagagccgtgtttattgagtcggtattcagagtctgaccagaaattattaatctggtgaagtttttcctctgtcattacgtcatggtcgatttcaatttctattgatgctttccagtcgtaatcaatgatgtattttttgatgtttgacatctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaataaaggccactatcaggcagctttgttgttctgtttaccaagttctctggcaatcattgccgtcgttcgtattgcccatttatcgacatatttcccatcttccattacaggaaacatttcttcaggcttaaccatgcattccgattgcagcttgcatccattgcatcgcttgaattgtccacaccattgatttttatcaatagtcgtagtcatacggatagtcctggtattgttccatcacatcctgaggatgctcttcgaactcttcaaattcttcttccatatatcaccttaaatagtggattgcggtagtaaagattgtgcctgtcttttaaccacatcaggctcggtggttctcgtgtacccctacagcgagaaatcggataaactattacaacccctacagtttgatgagtatagaaatggatccactcgttattctcggacgagtgttcagtaatgaacctctggagagaaccatgtatatgatcgttatctgggttggacttctgcttttaagcccagataactggcctgaatatgttaatgagagaatcggtattcctcatgtgtggcatgttttcgtctttgctcttgcattttcgctagcaattaatgtgcatcgattatcagctattgccagcgccagatataagcgatttaagctaagaaaacgcattaagatgcaaaacgataaagtgcgatcagtaattcaaaaccttacagaagagcaatctatggttttgtgcgcagcccttaatgaaggcaggaagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatctgaattgctatgtttagtgagttgtatctatttatttttcaataaatacaattggttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcatgtagccgcttatgctggaaagaagcaataacccgcagaaaaacaaagctccaagctcaacaaaactaagggcatagacaataactaccgatgtcatatacccatactctctaatcttggccagtcggcgcgttctgcttccgattagaaacgtcaaggcagcaatcaggattgcaatcatggttcctgcatatgatgacaatgtcgccccaagaccatctctatgagctgaaaaagaaacaccaggaatgtagtggcggaaaaggagatagcaaatgcttacgataacgtaaggaattattactatgtaaacaccaggcatgattctgttccgcataattactcctgataattaatccttaactttgcccacctgccttttaaaacattccagtatatcacttttcattcttgcgtagcaatatgccatctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttcggccgatgaaatgcatatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgaagctttagagcgatttatcttctgaaccagactcttgtcatttgttttggtaaagagaaaagtttttccatcgattttatgaatatacaaataattggagccaacctgcaggtgatgattatcagccagcagagaattaaggaaaacagacaggtttattgagcgcttatctttccctttatttttgctgcggtaagtcgcataaaaaccattcttcataattcaatccatttactatgttatgttctgaggggagtgaaaattcccctaattcgatgaagattcttgctcaattgttatcagctatgcgccgaccagaacaccttgccgatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatgtactaaggaggttgtatggaacaacgcataaccctgaaagattatgcaatgcgctttgggcaaaccaagacagctaaagatctcggcgtatatcaaagcgcgatcaacaaggccattcatgcaggccgaaagatttttttaactataaacgctgatggaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatctaaggaaatacttacatatggttcgtgcaaacaaacgcaacgaggctctacgaatcgagagtgcgttgcttaacaaaatcgcaatgcttggaactgagaagacagcggaagctgtgggcgttgataagtcgcagatcagcaggtggaagagggactggattccaaagttctcaatgctgcttgctgttcttgaatggggggtcgttgacgacgacatggctcgattggcgcgacaagttgctgcgattctcaccaataaaaaacgcccggcggcaaccgagcgttctgaacaaatccagatggagttctgaggtcattactggatctatcaacaggagtcattatgacaaatacagcaaaaatactcaacttcggcagaggtaactttgccggacaggagcgtaatgtggcagatctcgatgatggttacgccagactatcaaatatgctgcttgaggcttattcgggcgcagatctgaccaagcgacagtttaaagtgctgcttgccattctgcgtaaaacctatgggtggaataaaccaatggacagaatcaccgattctcaacttagcgagattacaaagttacctgtcaaacggtgcaatgaagccaagttagaactcgtcagaatgaatattatcaagcagcaaggcggcatgtttggaccaaataaaaacatctcagaatggtgcatccctcaaaacgagggaaaatcccctaaaacgagggataaaacatccctcaaattgggggattgctatccctcaaaacagggggacacaaaagacactattacaaaagaaaaaagaaaagattattcgtcagagaattctggcgaatcctctgaccagccagaaaacgacctttctgtggtgaaaccggatgctgcaattcagagcggcagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaaactccgcgataagtggacccaactcgaaatcaaccgtaacaagcaacaggcaggcgtgacagccagcaaaccaaaactcgacctgacaaacacagactggatttacggggtggatctatgaaaaacatcgccgcacagatggttaactttgaccgtgagcagatgcgtcggatcgccaacaacatgccggaacagtacgacgaaaagccgcaggtacagcaggtagcgcagatcatcaacggtgtgttcagccagttactggcaactttcccggcgagcctggctaaccgtgaccagaacgaagtgaacgaaatccgtcgccagtgggttctggcttttcgggaaaacgggatcaccacgatggaacaggttaacgcaggaatgcgcgtagcccgtcggcagaatcgaccatttctgccatcacccgggcagtttgttgcatggtgccgggaagaagcatccgttaccgccggactgccaaacgtcagcgagctggttgatatggtttacgagtattgccggaagcgaggcctgtatccggatgcggagtcttatccgtggaaatcaaacgcgcactactggctggttaccaacctgtatcagaacatgcgggccaatgcgcttactgatgcggaattacgccgtaaggccgcagatgagcttgtccatatgactgcgagaattaaccgtggtgaggcgatccctgaaccagtaaaacaacttcctgtcatgggcggtagacctctaaatcgtgcacaggctctggcgaagatcgcagaaatcaaagctaagttcggactgaaaggagcaagtgtatgacgggcaaagaggcaattattcattacctggggacgcataatagcttctgtgcgccggacgttgccgcgctaacaggcgcaacagtaaccagcataaatcaggccgcggctaaaatggcacgggcaggtcttctggttatcgaaggtaaggtctggcgaacggtgtattaccggtttgctaccagggaagaacgggaaggaaagatgagcacgaacctggtttttaaggagtgtcgccagagtgccgcgatgaaacgggtattggcggtatatggagttaaaagatgaccatctacattactgagctaataacaggcctgctggtaatcgcaggcctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacg

SEQ ID NO: 16 - RNA, 72 nt, synthetic construct
  source 1..72: mol_type = other RNA
tcctcgttagtatagtggttagtatccccgcctgtcacgcgggagaccggggttcaattccccgacggggag

SEQ ID NO: 17 - DNA, 71 bp, synthetic construct
  source 1..71: mol_type = other DNA
gcattggtggttcagtggtagaattctcgcctcccacgcgggagacccgggttcaattcccggccaatgca